(19) United States
(12) Patent Application Publication
(10) Pub. No.: US 2024/0385173 A1
(43) Pub. Date: Nov. 21, 2024

(54) METHODS AND APPARATUS FOR TESTING IMMUNO-ONCOLOGY DRUGS AND BIOLOGICS USING MICROORGANOSPHERES

(71) Applicant: Xilis, Inc., Durham, NC (US)

(72) Inventors: Xiling SHEN, Durham, NC (US); Shengli DINGL, Durham, NC (US)

(21) Appl. No.: 18/692,489

(22) PCT Filed: Sep. 16, 2022
(86) PCT No.: PCT/IB2022/058785

§ 371 Date: Mar. 15, 2024

Related U.S. Application Data
(60) Provisional application No. 63/245,188, filed on Sep. 16, 2021.
(60) Provisional application No. 63/245,193, filed on Sep. 16, 2021.

(51) Int. Cl. G01N 33/50; C12N 5/09

(52) U.S. Cl. CPC G01N 33/5011; C12N 5/0693; G01N 33/5088; C12N 2503/02; C12N 2509/00; C12N 2533/54

(57) ABSTRACT

The present disclosure relates to a method for testing immuno-oncology drugs and biologics using microorganospheres (MOSs). The method comprises forming a plurality of MOSs from cancerous tumor biopsy tissue with such forming comprising dissociating cells from the cancerous tumor biopsy tissue and combining the dissociated cells with a fluid matrix material. The dissociating itself comprises an enzyme digestion protocol, mincing the cancerous tumor biopsy tissue, and incubating the cancerous tumor biopsy tissue in a digestion solution with agitation. The method also comprises testing at least one immuno-oncology drug or biologic using the plurality of MOSs within 10 days after forming the plurality of MOSs.

### FIELD OF THE DISCLOSURE

Systems and methods consistent with the present invention generally relate to microorganospheres (MOSs), and methods and apparatuses for forming and using MOSs. More particularly, in some embodiments, systems and methods consistent with the invention relate to the methods and apparatuses for forming and using MOSs that include a microenvironment (including immune cell microenvironment) similar to that of the source from which the MOSs originated. In some embodiments the MOSs are suitable for testing small molecule immune-oncology drugs and biologics.

### DISCUSSION OF THE FIELD

Model cell and tissue systems are useful for biological and medical research. The most common practice is to derive immortalized cell lines from tissue and culture them in two-dimensional (2D) conditions (e.g., in Petri dish or well plate). However, although immensely useful for basic research, 2D cell lines do not correlate well with individual patient response to therapy. In particular, three-dimensional cell culture models are proving particularly helpful in developmental biology, disease pathology, regenerative medicine, drug toxicity and efficacy testing, and personalized medicine. For example, spheroids and organoids are three-dimensional cell aggregates that have been studied. However, both traditionally formed organoids and spheroids have limitations that reduce their utility in certain applications.

Multicellular tumor spheroids were first described in the early 70s and obtained by culture of cancer cell lines under non-adherent conditions. Spheroids are typically formed from cancer cell lines as freely floating cell aggregates in ultra-low attachment plates. Spheroids have been shown to maintain more stem cell associated properties than 2D cell culture.

Organoids are in-vitro derived cell aggregates that include a population of stem cells that can differentiate into cells of major cell lineages. Organoids typically have a diameter of more than one mm, and are cultured through passages. It is typically slower to grow and expand organoid culture than 2D cell culture. To generate organoids from clinical samples, requires a sufficient number of viable cells (e.g., hundreds of thousands) to start with, so it is often challenging to derive organoids from low volume samples, such as from a biopsy, and—even if successful—it would take considerable time to expand the culture for applications such as drug testing. In addition, there is a large amount of variability in organoid size, shape and cell number. Organoids may require complex cocktails of growth factors and culture conditions in order to grow and express desired cell types.

Neither tumor spheroids nor organoids are optimal for rapid and reliable screening, particularly for personalized medicine, such as performing ex-vivo testing of drug response. For example, the practice of oncology continually faces an immense challenge of matching the right therapeutic regimen with the right patient, in addition to balancing relative benefit with risk to achieve the most favorable outcome. Patient-Derived Models of Cancer (PDMC) may include the use of organoids (including patient-derived organoids) to facilitate the identification and development of more individualized therapeutic targets. However, although retrospective studies have shown that organoids derived from resected or biopsied patient tumors correlate with patient response to therapy, there are major limitations in using organoids to guide therapy. As mentioned above, it takes months to derive and expand organoids, and particularly patient-derived organoids, from tumor samples for drug sensitivity tests, which decreases the clinical applicability, as patients cannot wait that long to receive treatment. In addition, the number of organoids needed to perform a drug screen with more than dozens of compounds currently cannot be obtained in a clinically feasible timeframe from a core biopsy specimen, which is often the only available form of tissue from patients with metastatic or inoperable cancer. The significant failure rate for deriving organoids from biopsies also prevents its use as a reliable diagnostic assay. Further, there may be a high degree of variability in the size (and potentially the response) of organoids, particularly with longer culture times, and therefore many passages.

Due to their better correlations with patient outcomes, PDMCs are also being exploited to replace 2D cell lines as a high-throughput screen platform for drug discovery, such as RNAi, CRISPR, and pharmacological small molecule screens. However, compared to cell lines, these PDMC models (including spheroids and organoids) are typically much slower to expand and manipulate, making it challenging and costly for high-throughput applications. The longer time required to expand these models to amplify the cell numbers also tends to allow the fastest growing clone in plastics to dominate and outcompete other clones, hence making the model more homogeneous and losing the original tissue compositions and clonal diversity. Furthermore, their relatively larger and heterogeneous sizes and limited diffusibility make them challenging for many automated fluorescence and imaging-based readout assays.

Thus, what is needed are methods, compositions and apparatuses for generating patient derived tissue models (e.g., tumor models and/or non-tumor tissue models) from resection or biopsies. In particular, it would be useful to provide methods and apparatuses that may enable a large number of patient-derived tissue models having predictable and clinically relevant properties from a single biopsy, such as an 18-gauge core biopsy, which could be completed within, e.g., 7-10 days after obtaining a biopsy. This would permit robust and reliable testing and minimize delays in guiding patient-specific therapies. Furthermore, it will also be useful to generate patient derived models that can expand quickly in a highly parallel manner, generating units with smaller and more uniform sizes, better controllability for cell number per unit, and better diffusibility (e.g., via increase surface to volume ratio), for high-throughput screen applications. Additionally, it would be useful to have better models for testing small molecule immuno-oncology (IO) drugs and biopharmaceuticals to give a more accurate indication of individual patient responses to such therapies. It would further be useful to have generated tumor units within MOSs that accurately reflect the immune microenvironment of the patient tissue, and that the viability of that reflected immune microenvironment persist for an extended period of time (so as to be incorporated in various testing).

### SUMMARY

Described herein are Micro-Organospheres (MOSs), apparatuses and methods of making MOSs, and apparatuses and methods of using MOSs. Also described herein are methods and systems for screening a patient using these MOSs, including personalized therapy methods.

In general, described herein are methods and apparatuses that form and grow MOSs containing cells originating from a patient, for example, extracted from a small patient biopsy, (e.g., for quick diagnostics to guide therapy), from resected patient tissue, including resected primary tumor or part of a dysfunctional organ (e.g., for high-throughput screening), and/or from already established PDMCs, including patient-derived xenografts (PDX) and organoids (e.g., to generate MOSs for high-throughput screening).

These MOSs may be formed from primary cells that are normal (e.g., normal organ tissue) or from tumor tissue. For example, in some embodiments, these methods and apparatuses may form MOSs from cancerous tumor biopsy tissue, enabling tailored treatments that can selected using the particular tumor tissue examined. Surprisingly, these methods and apparatuses permit the formation of hundreds, thousands or even tens of thousands (e.g., 500, 750, 1000, 2000, 5000, 10,000 or more) of MOSs from a single tissue biopsy, within a few hours of the biopsy being removed from the patient. Dissociated primary cells from the patient biopsy may be combined with a fluid matrix material, such as a substrate basement membrane matrix (e.g., MATRIGEL), to form the MOS. The resulting plurality of MOSs may have a predefined range of sizes (such as diameters, e.g., from 10 μm to 700 μm and any sub-range therewithin), and initial number of primary cells (e.g., between 1 and 1000, and in particular lower numbers of cells, such as between 1-200). The number of cells and/or the diameter may be controlled within, e.g., +/−5%, 10%, 15%, 20%, 25%, 30%, etc. These MOSs, when formed as described herein, have an exceptionally high survival rate (>75%, >80%, >85%, >90%, >95%) and are stable for use and testing within a very short period of time, including within the first 1-10 days after being formed (e.g., within 1 day, within 2 days, within 3 days, within 4 days, within 5 days, within 6 days, within 7 days, within 8 days, within 9 days, within 10 days, etc.). This allows for rapid tests on a potentially huge number of patient-specific and biologically relevant MOSs which may save critical time in developing and deploying a patient therapy, such as a cancer treatment plan. The MOSs described herein rapidly form 3D cellular structures that replicate and correspond to the tissue environment from which they were biopsied, such as a three-dimensional (3D) tumor microenvironment. The MOSs described herein may also be referred to as “droplets”. Each MOSs may include, e.g., as part of the fluid matrix material, growth factors and structural proteins (e.g., collagen, laminin, nidogen, etc.) that may mimic the original tissue (e.g., tumor) environment. Each MOS may also include immune cells of the original tissue. Virtually any primary cell tissue may be used, including virtually any tumor tissue.

For example, to date, all tumor types and sites tested have successfully produced MOSs (e.g., current success rate of 100%, n=32, including cancer of the colon, esophagus, skin (melanoma), uterus, bone (sarcoma), kidney, ovary, lung, and breast from the primary site or metastatic sites including liver, omentum, and diaphragm). The tissue types used to successfully generate MOSs may be metastasized from other locations. In some embodiments the MOSs described herein can be grown from fine needle aspirate (FNA) or from circulating tumor cells (CTCs), e.g., from a liquid biopsy. Proliferation and growth are typically seen in as few as 3-4 days, and the MOSs can be maintained and passaged for months, or they may be cryopreserved and/or used for assays immediately (e.g., within the first 7-10 days).

In particular, described herein are methods of forming Patient-Derived MOSs. In some embodiments, these methods include combining dissociated primary tissue cells (including, but not limited to cancer/abnormal tissue, normal tissue, etc.) with a liquid matrix material to form an unpolymerized material, and then polymerizing the unpolymerized material to form MOSs that are typically less than about 1000 μm (e.g., less than about 900 μm, less than about 800 μm, less than about 700 μm, less than about 600 μm, and in particular, less than about 500 μm) in diameter in which the dissociated primary tissue cells are distributed. The number of dissociated cells may be within a predetermined range, as mentioned above (e.g., between about 1 and about 500 cells, between about 1-200 cells, between about 1-150 cells, between about 100 cells, between about 1-75 cells, between about 1-50 cells, between 35 about 1-30 cells, between about 1-20 cells, between about 1-10 cells, between about 5-15 cells, between about 20-30 cells, between about 30-50 cells, between about 40-60 cells, between about 50-70 cell, between about 60-80 cells, between about 70-90 cells, between about 80-100 cells, between about 90-110 cells, etc., including about 1 cell, about 10 cells, about 20 cells, about 30 cells, about 40 cells, about 50 cells, about 60 cells, about 70 cells, etc.). Any of these methods may be configured as described herein to produce MOSs of repeatable size (e.g., having a narrow distribution of sizes), as well as MOS that include immune cells.

The dissociated cells may be freshly biopsied and may be dissociated in any appropriate manner, including mechanical and/or chemical dissociation (e.g., enzymatic disaggregation by using one or more enzymes, such as collagenase, trypsin, etc.). The dissociated cells may optionally be treated, selected and/or modified. For example, the cells may be sorted or selected to identify and/or isolate cells having one or more characteristics (e.g., size, morphology, etc.). The cells may be marked (e.g., with one or more markers) that may be used to aid in selection. In some embodiments the cells may be sorted by a known cell-sorting technology, including but not limited to microfluidic cell sorting, fluorescent activated cell sorting, magnetic activated cell sorting, etc. Alternatively, the cells may be used without sorting.

In some embodiments, the dissociated cells may be modified by treatment with one or more agents. For example, the cells may be genetically modified. In some embodiments the cells may be modified using CRISPR-Cas9 or other genetic editing techniques. In some embodiments the cells may be transfected by any appropriate method (e.g., electroporation, cell squeezing, nanoparticle injection, magnetofection, chemical transfection, viral transfection, etc.), including transfection with of plasmids, RNA, siRNA, etc. Alternatively, the cells may be used without modification.

One or more additional materials may be combined with the dissociated cells and fluid (e.g., liquid) matrix material to form the unpolymerized mixture. For example, the unpolymerized mixture may include additional cell or tissue types, including support cells. The additional cells or tissue may originate from different biopsy (e.g., primary cells from a different dissociated tissue) and/or cultured cells. The additional cells may be, for example immune cells, stromal cells, endothelial cells, etc. The additional materials may include medium (e.g., growth medium, freezing medium, etc.), growth factors, support network molecules (e.g., collagen, glycoproteins, extracellular matrix, etc.), or the like. In some embodiments the additional materials may include a drug composition. In some embodiments the unpolymerized mixture includes only the dissociated tissue sample (e.g., primary cells) and the fluid matrix material.

The methods may rapidly form a plurality of MOSs from a single tissue biopsy, so that greater than about 500 Patient-Derived MOSs are formed from per biopsy (e.g., greater than about 600, greater than about 700, greater than about 800, greater than about 900, greater than about 1000, greater than about 2000, greater than about 2500, greater than about 3000, greater than about 4000, greater than about 5000, greater than about 6000, greater than about 7000, greater than about 8000, greater than about 9000, greater than about 10,000, greater than about 11,000, greater than about 12,000, etc.). The biopsy may be a standard size biopsy, such as an 18 G (e.g., 14 G, 16 G, 18 G, etc.) core biopsy. For example, the volume of tissue removed by biopsy and used to form the plurality of MOSs may be a small cylinder (taken with a biopsy needle) of between about 1/32 and ⅛ of an inch diameter and about ¾ inch to ¼ inch long, such as a cylinder of about 1/16 inch diameter by ½ inch long. The biopsy may be taken by needle biopsy, e.g., by core needle biopsy. In some embodiments the biopsy may be taken by fine needle aspiration. Other biopsy types that may be used include shave biopsy, punch biopsy, incisional biopsy, excisional biopsy, and the like. Typically, the material from a single patient biopsy may be used to generate the plurality (e.g., greater than about 2000, greater than about 5000, greater than about 7500, greater than about 10,000, etc.) of MOSs, as described above. A single core biopsy may contain about 50,000 to 500,000 cells. The number of cells in a single core biopsy may depend on the quality of the biopsy and the nature of the tissue source. For immune-oncology drugs, each MOS may comprise 80 to 100 cells. For traditional chemo drugs each MOS may comprise 20 to 40 cells. The plurality of Patient-MOSs may be formed using an apparatus (as described herein) that may be configured to generate this large number of highly regular (size, cell number, etc.) MOSs as described herein. In some embodiments these methods and apparatuses may generate the plurality or MOSs at a rapid rate (e.g., greater than about 1 MOS per minute, greater than about 1 MOS per 10 seconds, greater than about 1 MOS per 5 seconds, greater than about 1 MOS per 2 seconds, greater than about 1 MOS per second, greater than about 2 MOSs per second, greater than about 3 MOSs per second, greater than about 4 MOSs per second, greater than about 5 MOSs per second, greater than about 10 MOSs per second, greater than 50 MOSs per second, greater than 100 MOSs per second, greater than 125 MOSs per second, etc.).

For example, in some embodiments, these methods may be performed by combing the unpolymerized mixture with a material (e.g., liquid material) that is immiscible with the unpolymerized material. The method and apparatus may control the size and/or cell density of the MOSs by, at least in part, controlling the flow of one or more of the unpolymerized mixture (and/or the dissociated tissue and fluid matrix) and the material that is immiscible with the unpolymerized mixture (e.g., a hydrophobic material, oil, etc.). For example, in some embodiments, these methods may be performed using a microfluidics apparatus. In some embodiments, multiple MOSs may be formed in parallel (e.g., 2 in parallel, 3 in parallel, 4 in parallel, etc.). The same apparatus may therefore include multiple parallel channels, which may be coupled to the same source of unpolymerized material, or the same source of dissociated primary tissue and/or a source of fluid matrix.

The unpolymerized material may be polymerized in order to form the MOSs in a variety of different ways. In some embodiments the methods may include polymerizing the MOSs by changing the temperature (e.g., raising the temperature above a threshold value, such as, for example greater than about 20 degrees C., greater than about 25 degrees C., greater than about 30 degrees C., greater than about 35 degrees C., etc.).

Once polymerized, the MOSs may be allowed to grow, e.g., by culturing and/or may be assayed either before or after culturing and/or may be cryopreserved either before or after culturing. The MOSs may be cultured for any appropriate length of time, but in particular, may be cultured for between 1 day and 10 days (e.g., between 1 day and 9 days, between 1 day and 8 days, between 1 day and 7 days, between 1 day and 6 days, between 3 days and 9 days, between 3 days and 8 days, between 3 days and 7 days, etc.). In some embodiments, the MOSs may be cryopreserved or assayed before six passages, which may preserve the heterogeneity of the cells within the MOSs; limiting the number of passages may prevent the faster-dividing cells from outpacing more slowly dividing cells.

In general, since the same patient biopsy may provide a high number (e.g., greater than 2,000, greater than 3,000, greater than 4,000, greater than 5,000, greater than 6,000, greater than 7,000, greater than 8,000, greater than 9,000, greater than 10,000, etc.) cells, some of the MOSs may be cryopreserved (e.g., over half) while some are cultured and/or assayed. As will be described in greater detail herein, cryopreserved MOSs may be banked and used (e.g., assayed, passaged, etc.) later.

Thus, described herein are methods, including methods of forming a plurality of MOSs. For example, a method of forming a plurality of MOSs may include: combining a dissociated tissue sample and a fluid matrix material to form an unpolymerized mixture; forming a plurality of droplets of the unpolymerized mixture; and polymerizing the droplets to form a plurality of MOSs each having a diameter of between 50 and 500 μm with between 1 and 200 dissociated cells distributed therein.

A method, e.g., of forming a plurality of MOSs, may include combining a dissociated tissue sample and a fluid matrix material to form an unpolymerized mixture; forming a plurality of droplets from a continuous stream of the unpolymerized mixture wherein the droplets have less than a 25% embodiment in size; and polymerizing the droplets by warming to form a plurality of MOSs each having between 1 and 200 dissociated cells distributed within each MOS.

In some embodiments, a method as described herein for forming a plurality of MOSs may include: combining a dissociated tissue sample and a fluid matrix material to form an unpolymerized mixture; forming a plurality of droplets having less than a 25% variation in size of the droplets by converging a stream of the unpolymerized mixture with one or more streams of a fluid that is immiscible with the unpolymerized mixture; polymerizing the droplets to form a plurality of MOSs having a diameter of between 50 and 500 μm with between 1 and 200 dissociated cells distributed therein; and separating the plurality of MOSs from the fluid that is immiscible.

Any of these methods may include modifying the cells within the dissociated tissue sample prior to forming the droplets.

Forming the plurality of droplets may comprise forming a plurality of droplets of the unpolymerized mixture of uniform size with less than about 25% variation in size (e.g., less than about 20% variation in size, less than about 15% variation in size, less than about 10% variation in size, less than about 8% variation in size, less than about 5% variation in size, etc.). The variations in size may also be described as a narrow distribution of size variation. For example, the distribution of sizes may include a MOS size distribution (e.g., MOS diameter vs. the number of formed MOSs) having a low standard deviation (e.g., a standard deviation of 15% or less, a standard deviation of 12% or less, a standard deviation of 10% or less, a standard deviation of 8% or less, a standard deviation of 6% or less, a standard deviation of 5% or less, etc.).

Any of these methods may also include plating or distributing the MOSs. For example, in some embodiments, the method may include combining MOSs from various sources into a receptacle prior to assaying. For example, the MOSs may be placed into a multi-well plate. Thus, any of these methods may include dispensing the MOSs into a multi-well plate prior to assaying the MOSs. One or more (or in some embodiments equal amounts of) MOSs may be included per well.

In some embodiments applying the MOSs into a receptacle may include placing the MOSs into a plurality of chambers that are separated by an at least partially permeable membrane to permit circulation of supernatant material between the chambers. This may allow the MOSs to share the same supernatant.

In any of these methods the MOSs may be assayed. An assay may generally include exposing or treating individual MOSs to a condition (e.g., drug compositions or combinations of drug compositions, including but not limited to any of the drug compositions described herein) to determine if the condition has an effect on the cells of the MOSs (and in some cases, what effect it has). Assays may include exposing a subset of the MOSs (individually or in groups) to one or more concentrations of a drug composition, and allowing the MOSs to remain exposed for a predetermined time period (minutes, hours, days, etc.) and/or exposing and removing the drug composition, then culturing the MOSs for a predetermined time period. Thereafter the MOSs may be examined to identify any effects, including in particular toxicity on the cells in the MOSs, or a change in morphology and/or growth of the cells in the MOSs. In some embodiments assaying may include marking (e.g., by immunohistochemistry) live or fixed cells within the MOSs. Cells may be assayed (e.g., examined) manually or automatically. For example, cells may be examined to determine any toxicity (cell death) using an automated reader apparatus. In some embodiments assaying the plurality of MOSs may include sampling one or more of a supernatant, an environment, and a microenvironment of the MOSs for secreted factors and other effects. In any of these embodiments, the MOSs may be recovered following the assay for further assaying, expansion, or preservation (e.g., cryopreserving, fixation, etc.) for subsequent examination.

As mentioned, virtually any assay may be used. For example, genomic, transcriptomic, proteomics, or meta-genomic markers (such as methylation) may be assayed using the MOSs described herein. Thus, any of these compositions and methods described herein may be used to identify or examine one or more markers and biological/physiological pathways, including, for example, exosomes, which may assist in identifying drugs and/or therapies for patient treatment.

Any appropriate tissue sample may be used. In some embodiments, the tissue sample may include a biopsy sample from a metastatic tumor. For example, a tissue sample may comprise a clinical tumor sample; the clinical tumor sample may comprise both cancer cells and stroma cells. In some embodiments, the tissue sample comprises tumor cells and one or more of: mesenchymal cells, endothelial cells, and immune cells.

Any of the methods described herein may include initially distributing the dissociated cells from the tissue biopsy uniformly, or in some embodiments non-uniformly, throughout the fluid matrix material, in any appropriate concentration. For example, in some embodiments, the methods described herein may include combining the dissociated tissue sample and the fluid matrix material so that the dissociated tissue cells are distributed within the fluid matrix material to a density of less than 1×10⁷ cells/ml (e.g., less than 9×10⁶ cells/ml, 7×10⁶ cells/ml, 5×10⁶ cells/ml, 3×10⁶ cells/ml, 1×10⁶ cells/ml, 9×10⁵ cells/ml, 7×10⁵ cells/ml, 5×10⁵ cells/ml, etc.).

In general, forming the droplet may comprise forming the droplet from a continuous stream of the unpolymerized mixture. For example, forming the droplet may comprise applying one or more convergent streams of a fluid that is immiscible with the unpolymerized mixture to the stream of unpolymerized mixture. The streams may be combined in a microfluidic device, e.g., a device having a plurality of converging channels into which the unpolymerized mixture and the immiscible fluid interact to form droplets having a precisely controlled volume. In some embodiments the droplets are formed (e.g., pinched off) in an excess of the immiscible material, and the droplets may be concurrently and/or subsequently polymerized to form the MOSs. For example, the region in which the streams converge may be configured to polymerize the unpolymerized mixture after the droplet has been formed, e.g., by heating, and/or the regions downstream may be configured to polymerize the unpolymerized mixture after the droplets have been formed and are surrounded by the immiscible material. In some embodiments the immiscible material is heated (or alternatively cooled) to a temperature that promotes polymerization of the unpolymerized material, forming the MOSs. For example, polymerizing may comprise heating the droplet to greater than 35 degrees C.

Thus, in any of these methods, forming the droplet may include forming the droplet in a fluid that is immiscible with the unpolymerized mixture. Further, any of these methods may include separating the immiscible fluid from the MOSs. Further, any of these methods may include removing the immiscible fluid from the MOSs. In general, an immiscible fluid may include a liquid (e.g., oil, polymer, etc.), including in particular a hydrophobic material or other material that is immiscible with the unpolymerized (e.g., aqueous) material.

The fluid matrix material may be a synthetic or non-synthetic unpolymerized basement membrane material. In some embodiments the unpolymerized basement material may comprise a polymeric hydrogel. In some embodiments the fluid matrix material may comprise a MATRIGEL. Thus, combining the dissociated tissue sample and the fluid matrix material may comprise combining the dissociated tissue sample with a basement membrane matrix.

The tissue sample may be combined with the fluid matrix material within six hours of removing the tissue sample from the patient or sooner (e.g., within about 5 hours, within about 4 hours, within about 3 hours, within about 2 hours, within about 1 hour, etc.).

Also described herein are methods of assaying or preserving MOSs. For example, a method may include: combining a dissociated tissue sample and a fluid matrix material to form an unpolymerized mixture; forming a plurality of droplets of the unpolymerized mixture having less than a 25% variation in a size of the droplets; polymerizing the droplets to form a plurality of MOSs having a diameter of between 50 and 700 μm with between 1 and 1000 dissociated cells distributed therein; and assaying or cryopreserving the plurality of MOSs.

In some embodiments a method may include: combining a dissociated tissue sample and a fluid matrix material to form an unpolymerized mixture; forming a plurality of droplets of the unpolymerized mixture; polymerizing the droplets to form a plurality of MOSs each having a diameter of between 50 and 500 μm with between 1 and 200 dissociated cells distributed therein; and cryopreserving or assaying the plurality of MOSs within 15 days, wherein the MOSs are assayed to determine the effect of one or more agents on the cells within the MOSs.

For example, a method may include: combining a dissociated tissue sample and a fluid matrix material to form an unpolymerized mixture; forming a plurality of droplets having less than a 25% variation in a size of the droplets by converging a stream of the unpolymerized mixture with one or more streams of a fluid that is immiscible with the unpolymerized mixture; polymerizing the droplets by warming to form MOSs each having a diameter of between 50 and 500 μm with between 1 and 200 dissociated cell distributed therein; and assaying or cryopreserving the MOSs before six passages, whereby heterogeneity of the cells within the MOSs is maintained, further wherein assaying comprises assaying in order to determine the effect of one or more agents on the cells within the MOSs.

In any of these methods, the plurality of MOSs may be cryopreserved or assayed before six passages, whereby heterogeneity of the cells within the MOSs is maintained. Any of these methods may further include modifying the cells within the dissociated tissue sample prior to forming the droplets.

Forming the droplet may include forming a plurality of droplets of the unpolymerized mixture of uniform size with less than about 25% variation in size (e.g., less than about 20%, less 35 than about 15%, less than about 10%, less than about 7%, less than about 5%, etc.).

Any of these methods may include culturing the MOSs for an appropriate length of time, as mentioned above (e.g., culturing the MOSs for between 2-14 days before assaying). For example, these methods may include removing the immiscible fluid from the MOSs before culturing. In some embodiments, culturing the MOSs comprises culturing the MOSs in suspension.

In general, assaying the MOSs may comprise genomically, transcriptomically, epigenomically and/or metabolically analyzing the cells in the MOSs before and/or after assaying or cryopreserving the MOSs. Any of these methods may include assaying the MOSs by exposing the MOSs to a drug (e.g., drug composition).

In any of these methods, assaying may comprise visually assaying the effect of the one or more agents on the cells in the MOSs either manually and/or automatically. Any of these methods may include marking or labeling cells in the MOSs for visualization. For example, assaying may include fluorescently assaying the effect of the one or more agents on the cells.

The MOSs described herein are themselves novel and may be characterized as a composition of matter. For example, a composition of matter may comprise a plurality of cryopreserved MOSs, wherein each MOS has a spherical shape having a diameter of between 50 μm and 500 μm and comprises a polymerized base material, and between about 1 and 1000 dissociated primary cells distributed within the base material that have been passaged less than six times, whereby heterogeneity of the cells within the MOSs is maintained. In some embodiments, the MOSs include cells of the immune system (“immune cells”).

Also described herein are compositions of matter comprising a plurality of cryopreserved MOSs, wherein each MOS has a spherical shape having a diameter of between 50 μm and 500 μm, wherein the MOSs have less than a 25% embodiment in size, and wherein each MOS comprises a polymerized base material, and between about 1 and 500 dissociated primary cells distributed within the base material that have been passaged less than six times, whereby heterogeneity of the cells within the MOSs is maintained. In some embodiments, the MOSs include immune cells from the tissue of origin.

The primary cells may be primary tumor cells. For example, the dissociated primary cells may have been genetically or biochemically modified. The plurality of cryopreserved MOSs may have a uniform size with less than 25% variation in size. In some embodiments the plurality of cryopreserved MOSs may comprise MOSs from various sources. In any of these MOSs, the majority of cells in each MOS may comprise cells that are not stem cells. In some embodiments, the primary cells comprise metastatic tumor cells. The primary cells may comprise both cancer cells and stroma cells. In some embodiments, the primary cells comprise tumor cells and one or more of: mesenchymal cells, endothelial cells, and immune cells.

The primary cells may be distributed within the polymerized base material at a density of less than, e.g., 5×10⁷ cells/ml, 1×10⁷ cells/ml, 9×10⁶ cells/ml, 7×10⁶ 10 cells/ml, 5×10⁶ cells/ml, 1×10⁶ cells/ml, 9×10⁵ cells/ml, 7×10⁵ cells/ml, 5×10⁵ cells/ml, 1×10⁵ cells/ml, etc.

In general, the polymerized base material may comprise a basement membrane matrix (e.g., MATRIGEL). In some embodiments the polymerized base material comprises a synthetic material.

The microoganoids may have a diameter of between 50 μm and 1000 μm, or more preferably between 50 μm and 700 μm, or more preferably between 50 μm and 500 μm, or between 50 μm and 400 μm, or between 50 μm and 300 μm, or between 50 μm and 250 μm, etc. (e.g., less than about 500 μm, less than about 400 μm, less than about 300 μm, less than about 250 μm, less than about 200 μm, etc.).

As mentioned, the MOSs described herein may include any appropriate number of primary tissue cells initially in each MOS, for example less than about 200 primary cells, or more preferably less than about 150 primary cells, or more preferably less than about 100 primary cells, or more preferably less than about 75 primary cells, or less than about 50 cells, or less than about 30 cells, or less than about 25 cells, or less than about 20 cells or less than about 10 cell, or less than about 5 cells, etc.). In some embodiments each MOS includes between about 1 and 500 cells, between about 1-400 cells, between bout 1-300 cells, between about 1-200 cells, between about 1-150 cells, between about 1-100 cells between about 1-75 cells, between about 30 1-50 cells, between about 1-30 cells, between about 1-25 cells, between about 1-20 cells, etc.

Also described herein are apparatuses for forming MOSs, and methods of operating these apparatuses to form the MOSs. For example, described herein are methods of operating a MOS forming apparatus comprising: receiving an unpolymerized mixture comprising a chilled mixture of a dissociated tissue sample and a first fluid matrix material in a first port; receiving a second fluid that is immiscible with the unpolymerized mixture in a second port; combining a stream of the unpolymerized mixture with one or more streams of the second fluid to form droplets of the unpolymerized mixture having a uniform size that varies by less than 25%; and polymerizing the droplets of the unpolymerized mixture to form a plurality of MOSs.

A method of operating a MOS forming apparatus may include: receiving an unpolymerized mixture comprising a chilled mixture of a dissociated tissue sample and a first fluid matrix material in a first port; receiving a second fluid that is immiscible with the unpolymerized mixture in a second port; combining a stream of the unpolymerized mixture at a first rate with one or more streams of the second fluid at a second rate to form droplets of the unpolymerized mixture having a uniform size that varies by less than 25%, wherein the droplets are between 50 μm and 500 μm diameter; and polymerizing the droplets of the unpolymerized mixture to form a plurality of MOSs.

Any of these methods may include coupling a first reservoir containing the unpolymerized mixture in fluid communication with the first port. For example, the method may include combining the dissociated tissue sample and the first fluid matrix material to form the unpolymerized mixture. In some embodiments, the method includes adding the unpolymerized mixture to a first reservoir in fluid communication with the first port. These methods may include coupling a second reservoir containing the second fluid in fluid communication with the second port. Any of these methods may include adding the second fluid to a second reservoir in fluid communication with the second port. In some embodiments, receiving the second fluid comprises receiving an oil.

In general, these methods may include separating the second fluid (e.g., the immiscible fluid) from the plurality of MOSs. This fluid may be manually or automatically separated. For example, the second (immiscible) fluid may be removed by washing, filtering, or any other appropriate method.

Combining the streams may comprise driving the stream of the unpolymerized mixture at a first flow rate across one or more streams of the second fluid which is traveling at a second flow rate. In some embodiments the first flow rate is greater than the second flow rate. Either or both the flow rate and/or the amount of material (e.g., the unpolymerized mixture) may be present in smaller amount than the second fluid, so that the unpolymerized mixture is encapsulated in a precisely-controlled droplet, as described herein, that may then be polymerized, e.g., within the second fluid. In some embodiments, the flow rate of the immiscible fluid is greater than the flow rate of the unpolymerized mixture.

In some embodiments, combining the streams comprises driving the stream of the unpolymerized mixture across a junction into which the one or more streams of the second fluid also converge. Polymerizing the droplets may comprise heating the droplets to greater than a temperature at which the unpolymerized material polymerizes (e.g., greater than about 20 degrees C., greater than about 25 degrees C., greater than about 30 degrees C., greater than about 35 degrees C., etc.). In some embodiments, the unpolymerized matrix material is of a type that can be polymerized with light or by a chemical reaction initiated on the MOS forming device.

Any of these methods may include aliquoting the plurality of MOSs. For example, aliquoting into a multi-well dish.

Also described herein are methods of treating a patient using these MOSs, and methods of assaying them. For example a method may include: receiving a patient biopsy from a tumor; and determining, within 2 weeks of taking of the biopsy, that the tumor will respond to a drug formulation by: forming, from the patient biopsy, a plurality of MOSs having a diameter of between 50 and 500 μm with between 1 and 200 dissociated tumor cells distributed through a polymerized base material, and exposing at least some of the MOSs to the drug formulation before the dissociated tumor cells have undergone more than five passages; and measuring an effect of the drug formulation on the cells within the at least some of the MOSs to determine if the drug will treat the tumor based on the determined effect.

In some embodiments, these methods may include determining that the tumor is still responding to the drug formulation after one or more administrations of the drug to the patient by receiving a second patient biopsy after the patient has been treated with the drug formulation and forming a second plurality of MOSs from the second patient biopsy, exposing at least some of the second plurality of MOSs to the drug formulation, and measuring the effect of the drug formulation on cells within the at least some of the second plurality of MOSs.

Determining that the tumor will respond to a drug formulation may include exposing at least some of the MOSs to a plurality of drug formulations, and reporting the measured effects for each of the drug formulations (e.g. cell death, cell growth rate, and other similar cellular characteristics). In some embodiments, determining further comprises dispensing the MOSs into a multi-well plate prior to assaying the MOSs.

Any of these methods may include biopsying the patient to collect the patient biopsy (or otherwise taking a tissue sample from a patient or a sample of patient-derived tissues or cells) and/or treating the patient with the drug formulation, or assisting a physician in treating the patient (e.g., advising the physician as to which drug formulations would be effective). In general, the time between receiving the biopsy and reporting may be less than about 21 days (e.g., less than about 15 days, less than about 14 days, less than about 13 days, less than about 12 days, less than about 11 days, less than about 10 days, less than about 9 days, less than about 8 days, less than about 7 days, etc.).

MOSs can be used for testing certain therapies that were previously difficult to test. Unlike in the formation of traditional bulk organoids, cells of the immune system (“immune cells”) present in the biopsied patient derived tissues (e.g., from a tumor) may also be present and persist in MOSs upon their formation, even after the extensive processing for MOS formation described herein. Immune cells in MOS prepared as described herein can persist for 7 days or more, and in some cases 14 days or more. In some embodiments immune cells may persist for 21 days or longer. Additionally, when using traditional bulk organoids, it can be difficult for some therapies, such as certain immune-oncology therapies and T cell biopharmaceuticals, to penetrate, reach and interact with patient derived tissues (e.g., from a tumor) within. In contrast, MOSs allow for penetration of those drugs much more readily.

Because MOS formation as described herein allows for immune cells from the patient derived tissues to be incorporated, the accuracy of testing the aforementioned drug formulations in MOSs is superior to testing in traditional bulk organoids, and the interactions between immune cells and tumor cells can be monitored. These interactions may confer useful information about the effect of a drug on the interface and/or interaction between immune cells and cancer cells. In addition, immune cells derived from a patient may be separately introduced into MOS that have already formed, because of ease of penetration. In some embodiments, it is desirable to have the immune microenvironment of MOSs match that of the patient biopsy as closely as possible. Patient derived tissues (e.g., from a tumor) will include a variety of immune cells that are natively produced by the patient's body. A patient's response to particular drug formulations (e.g., immune-oncology drugs and biologics) can be directly impacted by the immune cells present at the target site. MOSs produced as described herein can be advantageous for such drug testing, as the drugs described herein can be tested in MOSs that include immune cells from the tissue of origin upon formation, or MOSs that include immune cells that are introduced after MOSs are formed.

In some embodiments, testing of MOSs is improved if those MOSs include some of the same immune cells found in the target patient derived tissues. To achieve this, certain steps may be taken to ensure not only that the matching immune microenvironment is found in the MOSs, but also that the immune cells in that immune microenvironment survive the process of making MOSs so as to be viable during testing. In some embodiments, the immune cells in the MOS persist for 7-10 days or longer, 10-14 days or longer. In some embodiments, the immune cells in the MOS may persist for 21 days or longer. In some embodiments, the immune cells in the MOS may surprisingly persist when MOS is placed in media devoid of cytokines used to sustain immune cells. In some embodiments, the immune cells in the MOS persist in the absence of any cytokines in media in which the MOS are placed. In some embodiments, MOS are placed in the aforementioned media after MOS are formed, and while organoids develop, according to embodiments described herein.

In some embodiments, chunk preparation can be utilized to maximize persistence and/or maintenance of the immune cells in the MOSs. In some embodiments, this can be done by dissociating patient derived tissues (e.g., tumor tissue) with a collagenase-based enzyme digestion protocol. Some embodiments further include mincing patient derived tissue and incubating it in a digestion solution (which may be dependent on tissue type) with agitation. Chunks can then be identified in clumps of cells (e.g., 50-100 cells). These chunks can then be filtered into a MOSs generation system, as described herein (to avoid clogging), so that chunks are preserved in at least some of the MOSs.

Utilizing the chunk preparation allows for (i) rapid MOS generation (and consequently rapid testing, including drug screening described herein, within 0-10 days; (ii) observations of heterogeneity; and (iii) local immune or stromal components are kept in single organoids.

In some embodiments, the use of a membrane-based demulsification, (e.g., using a hydrophobic membrane to remove oil from the MOS) can be utilized to achieve persistence of the immune cells in the MOS.

For example, membrane-based demulsification involves contacting the MOSs against a hydrophobic membrane which may include passing the MOSs through a chamber (e.g., tube, channel, etc.) at least partially formed by a hydrophobic membrane. The chamber may comprise a tunnel or tube formed by the hydrophobic membrane. Negative pressure (e.g., vacuum) may optionally be applied on one side of the membrane, and/or the solution including the MOSs may be driven against the membrane. Alternatively, the MOSs may simply be contacted to the hydrophobic membrane, without requiring the application of force, including by applying negative pressure.

For example in some embodiments, contacting the MOSs against a hydrophobic membrane may include eluting the MOSs into a funnel formed by the hydrophobic membrane. Contacting the MOSs against a hydrophobic membrane may comprise filtering the MOSs against the hydrophobic membrane. In general, contacting the MOSs against a hydrophobic membrane may comprise passing a solution including the MOSs over the hydrophobic membrane.

Any appropriate porous hydrophobic surface, including but not limited to a hydrophobic “membrane” may be used. For example, the hydrophobic surface (e.g., membrane) may have a pore size that is between 0.1 and 5 μm (e.g., between about 0.1 and 2 μm, between about 0.2 and 4 μm, between about 0.1 and 1 μm, between about 0.3 and 3 μm, etc.). The surface texture of the porous hydrophobic surface may be rough rather than smooth.

In general, contacting the MOSs against a hydrophobic membrane may include retaining the MOSs in an aqueous medium. For example, the MOSs may be retained with an aqueous buffer/media on one side of the porous hydrophobic surface while the oil is wicked or removed into or through the porous hydrophobic surface.

In some embodiments, alternative methods of demulsification are used, for example, a demulsifying device that may make incorporate (i) magnetic separation or (ii) laminar flow properties and small microstructures to allow for gentle filtering of oil. Such alternative methods can also be utilized to achieve persistence of the immune cells in the MOS.

In some embodiments, it is desirable to utilize therapies derived from a patient's own immune cells. For example, autologous immune enhancement therapy allows immune cells (i) to be taken out from a patient's body, (ii) to be cultured and processed to activate them until their resistance to, for example, cancer is strengthened, and (iii) to be put back in the patient's body. Because such enhanced immune cells do not readily penetrate traditional bulk organoids, it can be difficult to test the efficacy of enhanced immune cells in vitro. However, the size and composition of MOSs are able to uptake such enhanced immune cells. Accordingly, MOSs can be used to test enhanced immune cells for efficacy in a patient and reduce the risk of subjecting patients to ineffective immune cell infusions and supplemental immune cell harvesting for additional infusions.

### BRIEF DESCRIPTION OF THE DRAWINGS

The accompanying drawings, which are incorporated in and constitute a part of this disclosure, illustrate various embodiments and aspects of the present invention. In the drawings:

FIGS. 1A to 1C illustrate Patient-Derived MOSs formed as described herein to include a single dissociated primary tissue cell per MOS, cultured for one day after forming (FIG. 1A), cultured for three days after forming (FIG. 1B), and cultured for seven days after forming (FIG. 1C). The cells originate from colorectal cancer (CRC) tissue.

FIGS. 2A to 2C illustrate Patient-Derived MOSs formed as described herein to include five dissociated primary tissue cells per MOS, cultured for one day after forming (FIG. 2A), cultured for three days after forming (FIG. 2B), and cultured for seven days after forming (FIG. 2C). The cells originate from colorectal cancer (CRC) tissue.

FIGS. 3A to 3C illustrate Patient-Derived MOSs formed as described herein to include twenty dissociated primary tissue cells per MOS, cultured for one day after forming (FIG. 3A), cultured for three days after forming (FIG. 3B), and cultured for seven days after forming (FIG. 3C). As in FIGS. 1A-1C and 2A-2C, the cells originate from colorectal cancer (CRC) tissue.

FIGS. 4A to 4E illustrate examples of Patient-Derived MOSs formed as described herein to include ten dissociated primary tissue cells per MOS. FIG. 4A shows the MOSs shortly after formation (at low magnification). FIG. 4B shows a higher magnification view of some of the MOSs of FIG. 4A taken after culturing for two days. FIG. 4C shows the MOSs after culturing for three days. FIG. 4D shows the MOSs after culturing for four days. FIG. 4E shows the MOSs after culturing for five days.

FIGS. 5A to 5B illustrate examples of MOSs formed as described herein from normal mouse liver hepatocytes, cultured for one day after forming (FIG. 1A), or cultured for ten days after forming (FIG. 1B). The mouse hepatocytes are taken from a normal (e.g., non-diseased) mouse liver.

FIG. 6 illustrates on the method of forming Patient-Derived MOSs from primary tissue (e.g., biopsy) samples, as described herein.

FIG. 7A schematically illustrates one example of an apparatus for forming Patient-Derived MOSs as described herein, including a microfluidic chip as part of the assembly. FIG. 7B is a perspective view of one example a microfluidics chip portion of an apparatus such as that shown in FIG. 7A. FIG. 7C schematically illustrates a portion of a microfluidics assembly for an apparatus for forming Patient-Derived MOSs, such as the one shown in FIG. 7A.

FIG. 8 shows one example of an image showing a plurality of Patient-Derived MOSs formed using an apparatus such as that shown in FIG. 7A, showing the Patient-Derived MOSs shortly after polymerizing, suspended within a channel containing the immiscible fluid (e.g., oil) prior to being aliquoted from the apparatus.

FIG. 9 is an image of a portion of a prototype microfluidics assembly for an apparatus for forming Patient-Derived MOSs, similar to that shown in FIG. 7C, illustrating the formation of Patient-Derived MOSs.

FIG. 10 illustrates a plurality of Patient-Derived MOSs as described herein, shortly after polymerization; the Patient-Derived MOSs are suspended in the immiscible fluid.

FIGS. 11A to 11B illustrate another example of a plurality of Patient-Derived MOSs shortly after formation and suspended in the immiscible fluid (e.g., oil) at low magnification (FIG. 11A) and higher magnification (FIG. 11B).

FIGS. 12A to 12B show a plurality of Patient-Derived MOSs following separation from the immiscible fluid within a few hours of formation of the Patient-Derived MOSs at low magnification (FIG. 12A) and higher magnification (FIG. 12B).

FIG. 13 is another example of an image showing a plurality of Patient-Derived MOSs formed as described herein.

FIG. 14 is a chart illustrating the size distribution of the diameters from a plurality of Patient-Derived MOSs formed from an exemplary biopsy sample.

FIGS. 15A to 15B illustrate low and higher magnification views, respectively, of one example of a plurality of Patient-Derived MOSs formed from a dissociated tissue biopsy sample and a fluid matrix material, after polymerizing. FIG. 15A is an unstained image, while in FIG. 15B the MOSs have been stained with Trypan blue to show that the dissociated cells in the MOSs are alive.

FIGS. 16A to 16B is another example, similar to that shown in FIGS. 15A-15B, showing low and higher magnification views, respectively, of one example of a plurality of Patient-Derived MOSs. FIG. 16A is an unstained image, while in FIG. 16B the MOSs have been stained with Trypan blue (arrows) to show that the dissociated cells in the MOSs indicated that the cell remail viable (e.g., living) within the MOS.

FIGS. 17A-17E illustrates one example of a method of assaying a plurality of Patient-Derived MOSs formed from a patient tumor biopsy, to determine a drug-response profile to multiple drug formulations. The illustrated procedure takes less than two weeks (e.g., approximately one week) from biopsy to results.

FIG. 18 schematically illustrates an example of a method for treating a patient including the formation and use of a plurality of Patient-Derived MOSs as part of the treatment procedure.

FIG. 19 schematically illustrates an example of a method for treating a patient including multiple iterations of rapidly forming and assaying a plurality of Patient-Derived MOSs as part of the treatment procedure.

FIG. 20 schematically illustrates one embodiment of a portion of an apparatus for forming a plurality of Patient-Derived MOSs as described herein.

FIG. 21 schematically illustrates a method of operating an apparatus for forming a plurality of Patient-Derived MOSs similar to that shown in FIG. 20.

FIGS. 22A to 22D illustrate one example of a validation of a method of using a plurality of Patient-Derived MOSs as described herein to identify drug resistance. FIG. 22A illustrates the use of traditional (“2D”) tumor cell assay methods for predicting drug resistance. FIG. 22B illustrates the use of one example of a Patient-Derived MOS method as described herein, to assay for drug resistance for predicting drug sensitivity. FIGS. 22C and 22D show that the Patient-Derived MOS based method accurately predicted the actual response of the tumor (drug responsive), unlike traditional cultured cells.

FIGS. 23A to 23D illustrate another example validating the use of Patient-Derived MOSs as described herein to identify drug resistance, showing the predicted drug response to both Oxaliplatin and Irinotecan as consistent with actual tumor response following treatment with these drugs.

FIG. 24 illustrates one example of a drug screen using the Patient-Derived MOSs as described herein, in which a single tumor biopsy may generate a plurality of nearly-identical MOSs in large quantities extremely fast (e.g., within less than two weeks) and be quickly tested against a large number of drug formulations (e.g., 27 are shown) in parallel.

FIGS. 25A to 25B illustrate examples of mouse liver MOSs formed from a mouse liver tissue, having diameters of 300 pm, and 1 cell per MOS. FIG. 25A shows the MOSs at day 1, and FIG. 25B shows the MOSs at day 10.

FIGS. 26A to 26B illustrate examples of mouse liver MOSs formed from the partial hepatectomy mouse liver tissue, having diameters of 300 pm, and 25 cells per MOS similar to those shown in FIGS. 25A-25B. FIG. 26A shows the MOSs at day 1, and FIG. 26B shows the MOSs at day 10.

FIGS. 27A to 27C illustrate examples of human liver MOSs formed from human liver tissue. FIG. 27A shows the MOSs at day 1, seeded with 40 cells/droplet.

FIGS. 27B and FIG. 27C show the MOSs at day 18. In FIG. 27B the MOSs are hepatocyte-like structures, while FIG. 27C shows Cholangiocyte-like MOSs.

FIGS. 28A to 28D show examples of MOSs generated from a patient derived xenograft tumor line, having diameters of 300 pm, and 1 cell per MOS. FIG. 28A shows the MOSs at day 1, FIG. 28B shows the MOSs at day 3, FIG. 28C shows the MOSs at day 5 and FIG. 28D shows the MOSs at Day 7.

FIGS. 29A to 29D show examples of MOSs generated from a patient-derived xenograft model, having diameters of 300 pm, and 5 cell per MOS. FIG. 29A shows the MOSs at day 1, FIG. 29B shows the MOSs at day 3, FIG. 29C shows the MOSs at day 5 and FIG. 29D shows the MOSs at Day 7.

FIG. 30 is a graph comparing the responses of traditional organoids and MOSs formed from a colorectal cancer patient-derived organoid to Oxalipalatin, showing a comparable response for the traditional organoids and MOSs.

FIG. 31 is a graph comparing the responses of traditional organoids and MOSs formed from two colorectal cancer patient-derived xenograft models to SN38 (7-Ethyl-10-hydroxy-camptothecin), showing comparable responses.

FIG. 32 is a graph comparing the responses of traditional organoids and MOSs formed from a colorectal cancer patient-derived xenograft model to 5-FU (Fluorouracil), showing comparable responses.

FIGS. 33A and 33B show examples of toxicity assays using mouse liver MOSs. FIG. 33A shows that the sizes of the tissue in the mouse liver MOSs in the control group are relatively large (as indicated by the arrows). In contrast, in FIG. 33A, showing the acetaminophen (10 mM) treatment group, the tissue in most of the MOSs is smaller and contains many dead cells.

FIGS. 34A and 34B show examples of toxicity assays using human liver MOSs. FIG. 34A shows typical human liver MOSs observed in the control group including tissue structures (indicated by the arrows). FIG. 34B shows MOSs in an acetaminophen (10 mM) treatment group, showing atypical tissue structures (arrows) and debris.

FIGS. 35A through 35B show the presence of gel droplets in oil and a subsequent removal of oil. FIG. 35A shows an example of gel droplets in oil. FIG. 35B illustrates the gel droplets for which an antistatic gun was used to remove oil. FIG. 35C illustrates the use of a chemical demulsification agent, in this case PFO (10% PFO) used to remove oil from the gel droplets. FIG. 35D illustrates gel droplets that have been processed to remove the oil using a porous hydrophobic surface (e.g., membrane).

FIG. 36 illustrates one example of a general method of forming/and or processing (including removing oil) gel droplets using a porous hydrophobic surface.

FIG. 37A through 37C show illustrative embodiments of a demulsifier. FIG. 37A shows a schematic diagram of a first illustrative embodiment of a demulsifier. FIG. 37B shows a schematic diagram of a second illustrative embodiment of a demulsifier. FIG. 37C shows cross-sectional view of a third illustrative embodiment of a demulsifier.

FIGS. 38A and 38B respectively depict MOSs generated from chunk preparation on day 1 and day 2. As seen in FIGS. 38A and 37B, MOSs preserve the local microenvironment and spatial heterogeneity.

FIGS. 39A to 39C depict a single cell transcriptomics analysis of tumor tissue and MOSs. As seen in FIGS. 39A-39C, MOS preserves tumor stromal cells.

FIG. 40 illustrates imputed marker gene expression represented in various UMAPs.

FIGS. 41A to 41H are tables showing how various immune components are preserved over time in MOSs derived from different tissue samples.

FIG. 42 illustrates the preservation of immune cells in an established multiple myeloma biopsy.

FIGS. 43A and 43B respectively illustrate the effect of Nivolumab on immuno-oncology assays of MOSs based on pulmonary and renal tumors. FIG. 43A shows via Annexin V marker that Nivolumab induces apoptosis for a pulmonary tumor. FIG. 43B shows via Annexin V marker that Nivolumab induces apoptosis for a renal tumor.

FIGS. 44A and 44B illustrate the effect of Lenalidomine and Bortezoid on IO assays of MOSs based on a Multiple Myeloma (MM) biopsy. FIGS. 44A and 44B 36B show via Caspase 3/7 dye that Lenalidomine induces apoptosis for a MM biopsy while Bortezoid does not.

FIG. 45 illustrates the effect of ESK1 on organoid death in a pulmonary tumor MOS.

FIG. 46 illustrates the effect of ESK1 with added PBMC on organoid death in a pulmonary tumor MOS.

FIG. 47 illustrates the effect of patient TILs on tumor cells in MOS.

FIG. 48 illustrates the effect of PBMC on tumor cells in MOS.

FIG. 49 illustrates the effect of combining the treatment of Nivolumab with TILs.

FIG. 50 illustrates the ability of patient T cells to penetrate conventional bulk organoids vs. MOS.

### DETAILED DESCRIPTION

In general, described herein are MOSs, methods and apparatuses for forming them, and methods and apparatuses for using them, e.g., to assay for tissue (including, but not limited to cancerous tissue) responses.

The MOSs described herein are typically spheres formed from dissociated primary cells distributed within the base material. These MOSs may have a diameter of between about 50 μm and about 500 μm (e.g., between about 50 μm and about 400 μm, about 50 μm and about 300 μm, about 50 μm and about 250 μm, etc.), and may initially contain between about 1 and 1000 dissociated primary cells distributed within the base material (e.g., between about 1 and 750, between about 1 and 500, between about 1 and 400, between about 1 and 300, between about 1 and 200, between about 1 and 150, between about 1 and 100, between about 1 and 75, between about 1 and 50, between about 1 and 40, between about 1 and 30, between about 1 and 20, etc.).

Surprisingly, despite their small size (often between about 50-250 μm), and low cell density (e.g., often less than 100 cells per MOS), these MOSs may be used immediately or cultured for a very brief period of time (e.g., 14 days or less, 10 days or less, 7 days or less, 5 days or less, etc.) and may allow the cells within the MOSs to survive while maintaining much, if not all, of the characteristics of the tissue, including tumor tissue, from which they were extracted. The survival rate of the cells within the MOSs is remarkably high, and the MOSs may be cultured for days (or weeks) through multiple passages, in which the cells will divide, cluster and form structures similar to the parent tissue. Also surprisingly, in some embodiments, the cells from the dissociated tissue within the MOS form morphological structures inside even the smallest MOSs; although in some applications, the presence of such structures is not necessary for the utility of these MOSs (e.g., they may be used before substantial structural reorganization has occurred) in some embodiments they may be particularly useful.

The methods and apparatuses described herein for forming and using MOSs may be used to create many (e.g., greater than 10,000) MOSs from a single biopsy. These MOSs may be used screen for drug compositions that may predict what therapies may be effectively applied to the patient from whom the biopsy was taken. This may be useful, for example, in toxicity screen for drugs or other chemical compositions, from healthy normal tissue and/or from cancerous (e.g., tumor) tissue. In particular, the MOSs, methods and apparatuses for forming them and methods and apparatuses for testing them may be used for screening to identify one or more drug compositions or combinations of drug compositions that may effectively treat the patient (e.g., a cancer patient) prior to undergoing the drug therapy. This may allow, for example, very rapid screening of a cancer patient before they would otherwise undergo months of chemotherapy that may not be effective for them.

Thus, described herein are high-throughput drug screening methods (and apparatuses for performing these methods) using a single patient-specific biopsy (or other appropriate tissue/cell source). Described herein are droplet formed MOSs that may be formed from patient-derived tumor samples that have been dissociated and suspended in a basement matrix (e.g., MATRIGEL). The MOSs can be patterned onto a microfluidic microwell array, to be incubated, and dosed with drug compounds. This miniaturized assay maximizes the use of tumor samples, and enables more drug compounds to be screened from a core biopsy at much lower cost per sample.

Patient-derived models of cancer (PDMC), such as cell lines, organoids and patient-derived xenografts (PDXs) are increasingly being accepted as “standard” preclinical models to facilitate the identification and development of new therapeutics. For example, large-scale drug screens of cell lines and organoids derived from cancer patients have been used to identify sensitivity to a large number of potential therapeutics. PDXs are also used to predict drug response and identify novel drug combinations. Although precision medicine strategies are in development through the exploration of these various PDMC models, there are substantial barriers to their effective use. For example, patient derived organoids (PDO) are believed to be the most accurate in depicting patient tumors, as studies have shown that phenotypic and genotypic profiling of organoids often show a high degree of similarity to the original patient tumors. Unfortunately, at least two limitations hinder the use of PDO to guide therapy. Firstly, it takes several months to develop and test drug sensitivity in organoids, which decreases the clinical applicability. Secondly the number of organoids obtained from a clinically relevant 18-gauge core biopsy is not sufficient to perform high throughput drug screen. Ideally, an assay should be performed from a single core biopsy within 7-10 days. The MOSs and methods of making and using them described herein may address these clinical limitations.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

The term “an unpolymerized mixture” is used herein to refer to a composition comprising biologically-relevant materials, including a dissociated tissue sample and a first fluid matrix material. The fluid matrix material is typically a material that may be polymerized to form a support or support network for the dissociated tissue (cells) dispersed within it. Once polymerized, the polymerized material may form a hydrogel and may be formed or and/or may include proteins forming the biocompatible medium, in addition to the cells. A suitable biocompatible medium for use in accordance with the presently-disclosed subject matter can typically be formed from any biocompatible material that is a gel, a semi-solid, or a liquid, such as a low-viscosity liquid, at room temperature (e.g., 25° C.) and can be used as a three-dimensional substrate for cells, tissues, proteins, and other biological materials of interest. Exemplary materials that can be used to form a biocompatible medium in accordance with the presently-disclosed subject matter include, but are not limited to, polymers and hydrogels comprising collagen, fibrin, chitosan, MATRIGEL™ (BD Biosciences, San Jose, Calif.), polyethylene glycol, dextrans including chemically crosslinkable or photo-crosslinkable dextrans, and the like, as well as electrospun biological, synthetic, or biological-synthetic blends. In some embodiments, the biocompatible medium is comprised of a hydrogel.

The term “hydrogel” is used herein to refer to two- or multi-component gels comprising a three-dimensional network of polymer chains, where water acts as the dispersion medium and fills the space between the polymer chains. Hydrogels used in accordance with the presently-disclosed subject matter are generally chosen for a particular application based on the intended use of the structure, taking into account the parameters that are to be used to form the MOSs, as well as the effect the selected hydrogel will have on the behavior and activity of the biological materials (e.g., cells) incorporated into the biological suspensions that are to be placed in the structure. Exemplary hydrogels of the presently-disclosed subject matter can be comprised of polymeric materials including, but not limited to: alginate, collagen (including collagen types I and VI), elastin, keratin, fibronectin, proteoglycans, glycoproteins, polylactide, polyethylene glycol, polycaprolactone, polycolide, polydioxanone, polyacrylates, polyurethanes, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof as well as inorganic materials such as glass such as bioactive glass, ceramic, silica, alumina, calcite, hydroxyapatite, calcium phosphate, bone, and combinations of all of the foregoing.

With further regard to the hydrogels used to produce the MOSs described herein, in some embodiments, the hydrogel is comprised of a material selected from the group consisting of agarose, alginate, collagen type I, a polyoxyethylene-polyoxypropylene block copolymer (e.g., Pluronic® F127 (BASF Corporation, Mount Olive, N.J.)), silicone, polysaccharide, polyethylene glycol, and polyurethane. In some embodiments, the hydrogel is comprised of alginate.

The MOSs described herein may also include biologically-relevant materials. The phrase “biologically-relevant materials” may describe materials that are capable of being included in a biocompatible medium as defined herein and subsequently interacting with and/or influencing biological systems. For example, in some implementations, the biologically-relevant materials are magnetic beads (i.e., beads that are magnetic themselves or that contain a material that responds to a magnetic field, such as iron particles) that can be combined as part of the unpolymerized material to produce MOSs that can be used in the methods and compositions (e.g., for the separation and purification of MOSs). As another example, in other implementations, the biologically-relevant materials may include additional cells, in addition to the dissociated tissue sample (e.g., biopsy) material. In the unpolymerized mixture the dissociated tissue sample and the additional biologically relevant material can exist in a uniform mixture or as a distributed mixture (e.g., on just one half or other portion of the MOS, including just in the core or just in the outer region of the formed MOSs). In some embodiments the additional biologically-relevant material within the unpolymerized material may be suspended with the dissociated tissue sample in suspension, e.g., prior to polymerization of the droplet forming the MOS.

In some embodiments the biologically relevant material that may be included with the dissociated tissue sample (e.g., biopsy) material may contain a number of cell types, including preadipocytes, mesenchymal stem cells (MSCs), endothelial progenitor cells, T cells, B cells, mast cells, and adipose tissue macrophages, as well as small blood vessels or microvascular fragments found within the stromal vascular fraction.

In general, with respect to the dissociated tissue sample, e.g., biopsy, material that is included in the MOSs described herein, these tissues may be any appropriate tissue from a patient, typically taken by biopsy. Although non-biopsy tissue may be used, in general, these tissues (and the resulting dissociated cells) may be primary cells taken from a patient biopsy as described above, e.g., by a needle biopsy. Tissues may be from a healthy tissue biopsy or from cancerous (e.g., tumor) cell biopsy. The dissociated cells may be incorporated into a MOS of the presently-disclosed subject matter, based on the intended use of that MOS. For example, relevant tissues (e.g., dissociated biopsy tissue) may typically include cells that are commonly found in that tissue or organ (or tumor, etc.). In that regard, exemplary relevant cells that can be incorporated into MOSs of the presently-disclosed subject matter include neurons, cardiomyocytes, myocytes, chondrocytes, pancreatic acinar cells, islets of Langerhans, osteocytes, hepatocytes, Kupffer cells, fibroblasts, myoblasts, satellite cells, endothelial cells, adipocytes, preadipocytes, biliary epithelial cells, and the like. These types of tissues may be dissociated by conventional techniques known in the art. Suitable biopsied tissue can be derived from: bone marrow, skin, cartilage, tendon, bone, muscle (including cardiac muscle), blood vessels, corneal, neural, brain, gastrointestinal, renal, liver, pancreatic (including islet cells), lung, pituitary, thyroid, adrenal, lymphatic, salivary, ovarian, testicular, cervical, bladder, endometrial, prostate, vulval and esophageal tissue. Normal or diseased (e.g., cancerous) tissue may be used. In some embodiments, the tissue may arise from tumor tissue, including tumors originating in any of these normal tissues.

Once formed the MOSs may be cryopreserved and/or cultured. Cultured MOSs may be maintained in suspension, either static (e.g., in a well, vial, etc.) or in motion (e.g., rolling or agitated). The MOSs may be cultured using known culturing techniques. Exemplary techniques can be found in, among other places; Freshney, Culture of Animal Cells, A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022.

In some embodiments the MOSs are formed by forming a droplet of the unpolymerized mixture (e.g., in some embodiments a chilled mixture) of a dissociated tissue sample and a fluid matrix material in an immiscible material, such as a fluid hydrophobic material (e.g., oil). For example, a MOS may be formed by combining a stream of unpolymerized material with one or more streams of the immiscible material to form a droplet. The density of the cells present in the droplet may be determined by the dilution of the dissociated material (e.g., cells) in the unpolymerized material. The size of the MOSs may correlate to the size of the droplet formed. In general, the MOS is a spherical structure having a stable geometry.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, 15 D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor 20 Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

As used herein a drug composition may include any drug, drug dilution, drug formulation, compositions including multiple drugs (e.g., multiple active ingredients), drug formulations, drug forms, drug concentrations, combination therapies, and the like. In some embodiments a drug formulation refers to a formulation comprising a mixture of a drug and one or more inactive ingredients. As used herein the term “passaged” may refer to the average number of doublings of the cells within the MOSs. Although traditional passage number refers to the transfer or subculture of cells from one culture vessel to another, the cells within a MOS may be stably retained within the same MOS, and may continue to grow and divide. Thus, the passage number as used herein typically refers to the average number of doublings undergone by the dissociated cells from the biopsied tissue within the MOSs. The population doubling number is the approximate number of doublings that the cell population has undergone since isolation (e.g., since forming of the MOSs from the freshly dissociated biopsy tissue). In general, the MOSs described herein may be cultured for a short period of time relative to the growth, e.g., doublings, of some or all of the cells within the MOSs (e.g., fewer than 10 passages, fewer than 9 passages, fewer than 8 passages, fewer than 7 passages, fewer than 6 passages, fewer than 5 passages, fewer than 4 passages, fewer than 3 passages, etc.).

During culturing, the cells from the dissociated, biopsied tissue in the MOSs can aggregate, cluster, or assemble within the MOSs. Aggregates of cells may be highly organized, and may form defined morphology or may be a mass of cells that have clustered or adhered together. The organization may reflect the tissue of origin. Although in some embodiments the MOSs may contain a single cell type (homotypic), more typically these MOSs may contain more than one cell type (heterotypic).

As mentioned, the (e.g., biopsy) tissue used to form the MOSs (e.g., the dissociated tissue) may be derived from a normal or healthy biological tissue, or from a biological tissue afflicted with a disease or illness, such as a tissue or fluid derived from a tumor. The tissue used in the MOSs may include cells of the immune system, such as T lymphocytes, B lymphocytes, polymorphonuclear leukocytes, macrophages, natural killer cells, and dendritic cells. The cells may be stem cells, progenitor cells or somatic cells. As described herein, steps may be taken to enhance the ability of these cells of the immune system to viably persist longer. As described in further detail below, the presence of these immune cells can be used to enhance the efficacy and accuracy of drug/biologic testing. The tissue may be mammalian cells such as human cells or cells from animals such as mice, rats, rabbits, and the like.

In general, the tissue (and resulting cells) may generally be taken from a biopsy to form the MOSs. Thus, the tissue may be derived from any of a biopsy, a surgical specimen, an aspiration, a drainage, or a cell-containing fluid. Suitable cell-containing fluids include any of blood, lymph, sebaceous fluid, urine, cerebrospinal fluid or peritoneal fluid. For example, in patients with transcoelomic metastasis, ovarian or colon cancer cells may be isolated from peritoneal fluid. Similarly, in patients with cervical cancer, cervical cancer cells may be taken from the cervix, for example by large excision of the transformation zone or by cone biopsy. Typically, such MOSs will contain multiple cell types that are resident in the tissue or fluid of origin. The cells may be obtained directly from the subject without intermediate steps of subculture, or they may first undergo an intermediate culturing step to produce a primary culture. Methods for harvesting cells from biological tissue and/or cell containing fluids are well known in the art. For example, techniques used to obtain cells from biological tissue include those described by R. Mahesparan (Extracellular matrix-induced cell migration from glioblastoma biopsy specimens in vitro. Acta Neuropathol (1999) 97:231-239).

Generally, the cells are first dissociated or separated from each other before forming the MOSs. Dissociation of cells may be accomplished by any conventional means known in the art. Preferably, the cells are treated mechanically and/or chemically, such as by treatment with enzymes. By ‘mechanically’ we include the meaning of disrupting connections between associated cells, for example, using a scalpel or scissors or by using a machine such as an homogenizer. By ‘enzymatically’ we include the meaning of treating the cells with one or more enzymes that disrupt connections between associated cells, including for example any of collagenase, dispases, DNAse and/or hyaluronidase. One or more enzymes may be used under different reaction conditions, such as incubation at 37° C. in a water bath or at room temperature.

The dissociated tissue may be treated to remove dead and/or dying cells and/or cell debris. The removal of such dead and/or dying cells may be accomplished by any conventional means known to those skilled in the art, for example using beads and/or antibody methods. It is known, for example, that phosphatidylserine is redistributed from the inner to outer plasma membrane leaflet in apoptotic or dead cells. The use of Annexin V-Biotin binding followed by binding of the biotin to streptavidin magnetic beads enables the separation of apoptotic cells from living cells. Similarly, removal of cell debris may be achieved by any suitable technique in the art, including, for example, filtration.

The dissociated cells may be suspended in a carrier material prior to combining with the fluid matrix material, and/or the fluid matrix material may be referred to as a carrier material. In some embodiments the carrier material may be a material that has a viscosity level that delays sedimentation of cells in a cell suspension prior to polymerization and formation of the MOSs. A carrier material may have sufficient viscosity to allow the dissociated biopsy tissue cells to remain suspended in the suspension until polymerization. The viscosity required to achieve this can be optimized by the skilled person by monitoring the sedimentation rate at various viscosities and selecting a viscosity that gives an appropriate sedimentation rate for the expected time delay between loading the cell suspension into the apparatus forming the MOSs by polymerizing the droplets of the unpolymerized material including the cells. In some embodiments the unpolymerized material may be flowed or agitated by the apparatus even where lower viscosity materials are used, in order to keep the cells in suspension and/or distributed as desired.

As mentioned above, in some embodiments the unpolymerized mixture, including the dissociated tissue sample and the fluid matrix material may include one or more components, e.g., biologically-relevant materials. For example, a biologically-relevant material that may be included may be any of: an extracellular matrix protein (e.g. fibronectin), a drug (e.g. small molecules), a peptide, or an antibody (e.g., to modulate any of cell survival, proliferation or differentiation); and/or an inhibitor of a particular cellular function. Such biologically-relevant materials may be used, for example, to increase cell viability by reducing cell death and/or activation of cell growth/replication or to otherwise mimic the in vivo environment. The biologically-relevant materials may include or may mimic one or more of the following components: serum, interleukins, chemokines, growth factors, glucose, physiological salts, amino acids and hormones. For example, the biologically-relevant materials may supplement one or more agents in the fluid matrix material. In some embodiments, the fluid matrix material is a synthetic gel (hydrogel) and may be supplemented by one or more biologically-relevant materials. In some embodiments the fluid matrix is a natural gel. Thus, the gel may be comprised of one or more extracellular matrix components such as any of collagen, fibrinogen, laminin, fibronectin, vitronectin, hyaluronic acid, fibrin, alginate, agarose and chitosan. For example, MATRIGEL comprises bioactive polymers that are important for cell viability, proliferation, development and migration. For example, the matrix material may be a gel that comprises collagen type 1 such as collagen type 1 obtained from rat tails. The gel may be a pure collagen type 1 gel or may be one that contains collagen type 1 in addition to other components, such as other extracellular matrix proteins. A synthetic gel may refer to a gel that does not naturally occur in nature. Examples of synthetic gels include gels derived from any of polyethylene glycol (PEG), polyhydroxyethyl methacrylate (PHEMA), polyvinyl alcohol (PVA), polyethylene oxide (PEO).

### MOSs

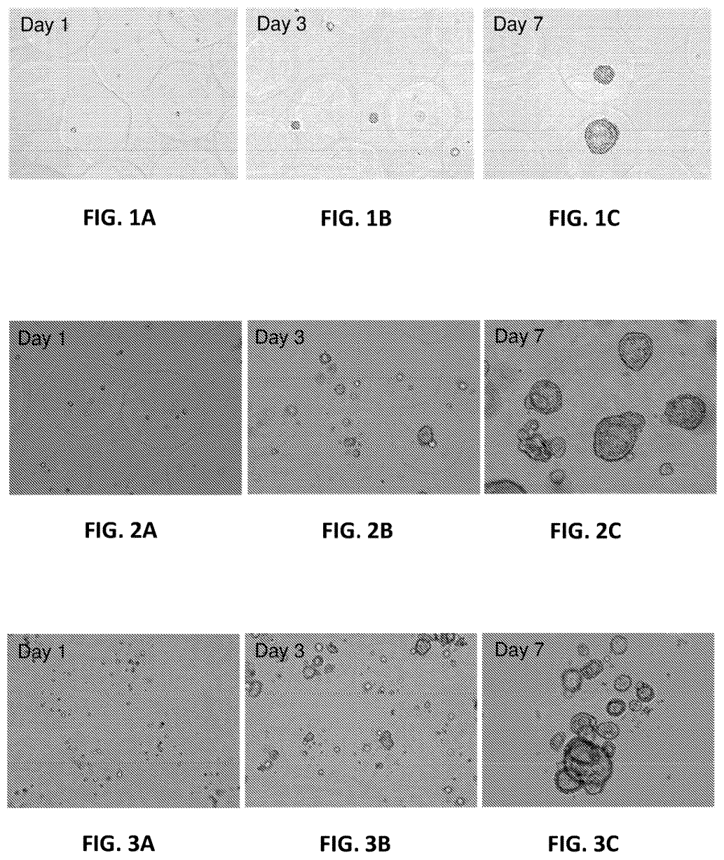

Examples of MOSs are shown in FIGS. 1A-1C, 2A-2C, 3A-3C and 4A-4E. For example, FIGS. 1A-1C illustrate MOSs formed having a single cell per MOS. As shown, the MOSs are all approximately the same size, e.g., approximately 300 μm diameter. FIG. 1B shows MOSs formed at the same time after 3 days in culture. The cells have expanded in size, in some cases doubling and/or growing. By seven days in culture, as shown in FIG. 1C, the cells have doubled multiple times, showing clusters or masses of cells.

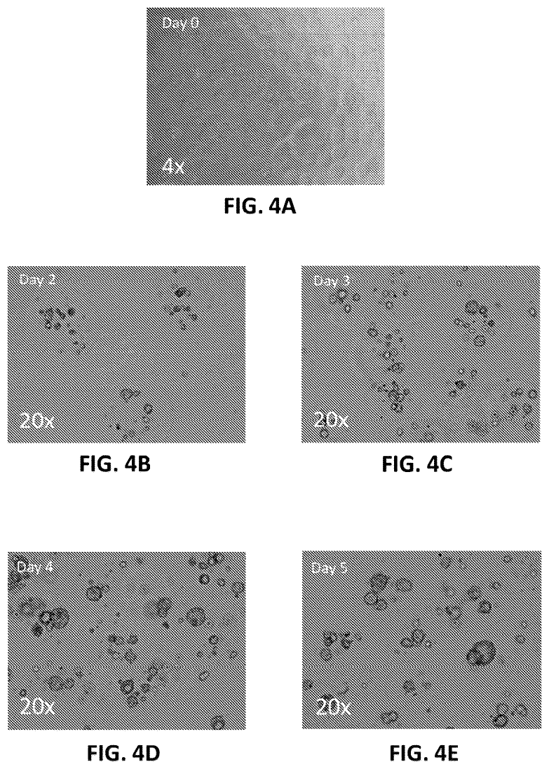

Similar results are shown in FIGS. 2A-2C and 3A-3C showing MOSs formed from five cells per MOS or 20 cells per MOS, respectively. In FIGS. 4A-4E, the MOSs are shown immediately after formation, and cultured for five days, in which nearly-identical MOSs (e.g., having the same diameter) each include 10 cells per MOS. In FIG. 4A, the MOSs are shown immediately after forming, still surrounded by the immiscible fluid, in this case, oil, at day 0. The MOSs are removed from the immiscible fluid and washed, and cultured for five days. FIG. 4B shows the MOSs after 2 days, FIG. 4C shows the MOSs after 3 days, and FIGS. 4D and 4E show the MOSs at 4 and 5 days, respectively. FIGS. 4A-4E show that the dissociated tissue (cells) from the biopsy within the MOSs are viable and growing within nearly all of the MOSs at comparable rates. As will be described in greater detail there, these MOSs may be formed in large amounts from even a single, average-sized biopsy and may result in many hundreds or thousands (e.g., 500, 750, 1000, 2000, 5000, 10,000 or more) of MOSs that include a significant number of viable cells, allowing multiple rapid assays to be performed in parallel.

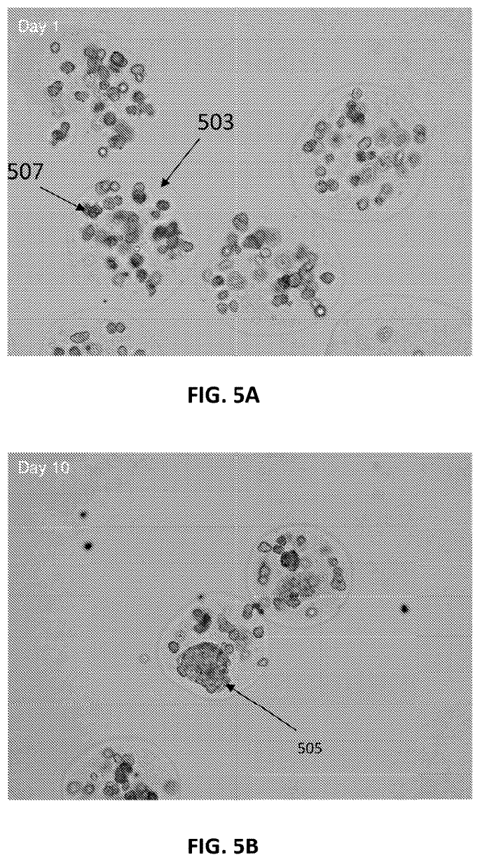

FIGS. 5A and 5B illustrate an example of MOSs formed as described herein from a dissociated biopsy of mouse liver, e.g., showing mouse hepatocytes distributed within a polymerized fluid matrix material (in this example, MATRIGEL). Each MOS includes the polymerized matrix material 503 formed into a sphere having a diameter, e.g., of about 300 μm, in which a set number of hepatocytes 507 are dispersed. In FIG. 5A, the MOSs are shown one day after biopsying, dissociation and forming of the MOSs. These MOSs were then cultured for 10 days, during which time the cells (hepatocytes) remained viable and grew, in many cases doubling multiple times to form structures 505, as shown in FIG. 5B.

The MOSs may generally include the dissociated (e.g., biopsy) tissue (e.g., cells) in a fixed or known number of cells and/or concentration (cells/ml or cells/mm3) within the MOSs. As mentioned above, this matrix material may be natural polymers, such as one or more of: alginate, agarose, hyaluronic acid, collagen, gelatin, fibrin, elastin; or a synthetic polymer, such as one or more of: polyethylene glycol (PEG) and polyacrylamide. Both organic and inorganic synthetic polymers may be used.

In some embodiments the number of cells initially included in the MOSs may be selected from between 1 cell up to several hundred. In particular, in some assays (e.g., drug toxicity assays) it may be beneficial to include between about 1-75 or between about 1-50 (e.g., lower numbers of cells). The number of cells per MOS may be set or selected by the user. In some embodiments, as described below, the apparatus will include one or more controls to set the number of cells from the primary tissue to include in each MOS. The number of cells may be chosen or set based on how the user intends to use the MOSs. For example, MOSs having very low number of cells (e.g., 1 cell per MOS, 1-5 cells per MOS, etc.) may be particularly suitable for studying clonal diversity (e.g., for tumor heterogeneity). Since each MOS grows from a single cell, we can observe which clones are drug resistant and these specific MOSs may be examined (e.g., by genomic sequencing) to determine the genomic (mutation) diversity related to the particular clone. A low to moderate number of cells per MOS (e.g., between about 3-30 cells, 5-30 cells, 5-25 cells, 5-20 cells, 10-25 cells, etc.) may be particularly useful for rapid drug testing, including toxicity testing as these MOSs typically grow quickly. A larger number of cells per MOS (e.g., between about 20-100 cells, e.g., 30-100 cells, 40-100 cells, greater than 50 cells, etc.) may be particularly suitable for mimicking tissue composition in each MOS, as the MOSs may contain different lineages, potentially including epithelial (or cancer, etc.) and mesenchymal (or stromal, immune, blood vessel, etc.) cells.

The MOSs may be formed in any appropriate size, which may be matched to the number of cells to be included. For example, the size may be as small as about 20 μm, up to 500 μm in diameter (e.g., 50 or 100 μm on average, e.g., between about 100-200 μm, etc.). In some embodiments the size is about 300 μm in which between about 10-50 cells (e.g., between about 10-30 cells) are included in each MOS. The number of cells and the size may be varied and/or may be controlled. In some embodiments the number of cells and/or the size of the MOSs may be set by one or more controls on the apparatus forming the MOSs. For example, the size of the MOSs and/or the density of cells within the MOSs may be adjusted by adjusting the flow rates and/or the concentration of the dissociated tissue sample (e.g., the cells from a biopsy).

As shown in FIGS. 1A-5B, even after culturing the MOSs described herein allow for viable and healthy cells through the entire volume of the MOS. The size of the MOSs and/or the number of cells to be included in the MOSs may be selected based on how the MOSs are expected or intended to be used. For example, in embodiments in which the MOSs are to be used to examine relationships between cells of the biopsied material the MOSs may be formed having multiple cells and may be cultured for extended periods of time (e.g., up to one week or more).

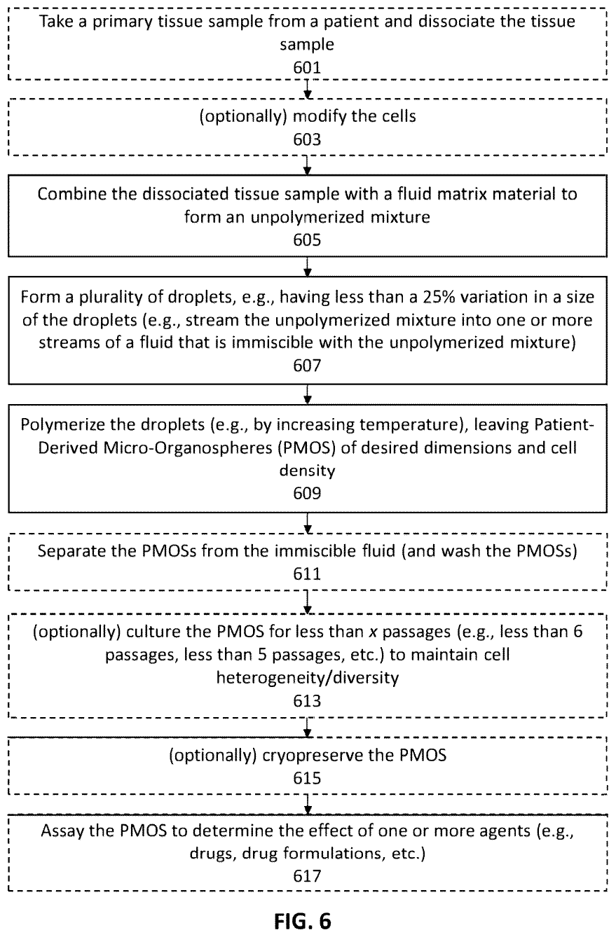

The MOSs described herein may be made by combining a dissociated tissue sample, e.g., a biopsy sample, with a fluid matrix that may be polymerized in a controlled manner (e.g., heat, light, or chemical crosslinking) to form the MOSs. FIG. 6 illustrates one method of forming MOSs.

Optionally, the method may include taking the sample from a patient, such as taking a biopsy from a patient tissue 601. As mentioned above, the biopsy may be taken, e.g., using a biopsy needle or punch. For example, the biopsy may be taken with a 14-gauge, a 16-gauge, an 18-gauge, etc. needle that is inserted into the patient tissue to remove the biopsy. After removing the tissue from the patient, the tissue may be processed to dissociate the material, either mechanically and/or chemically. The dissociated cells may be immediately used to form the MOSs, as described; in some embodiments, all or some of the cells may be modified, such as by genetically modifying the cells 603, for example, by transfection, electroporation, etc.

The dissociated tissue sample from the biopsy material may be combined with the fluid (e.g., liquid) matrix material to form the unpolymerized mixture 605. This unpolymerized mixture may be held in an unpolymerized state, so that the cells from the dissociated tissue may remain suspended within the mixture. In some embodiments the cell may remain suspected and unpolymerized by keeping them chilled, e.g., at room temperature of below (e.g., between 1-25 degrees C.).

The unpolymerized mixture may then be dispensed as droplets, e.g., into an immiscible material, such as an oil, in a manner that controls the formation of the size of the droplets and therefore the size of the MOSs formed 607. For example, uniformly-sized droplets may be formed by combining a stream of the unpolymerized material into one or more (e.g., two converging) streams of the immiscible material (e.g., oil) so that the flow rates and/or pressures of the two streams may determine how droplets of the unpolymerized material are formed as they intersect the immiscible material. The droplets may be polymerized 609 to form the MOSs in the immiscible material. In some embodiments the immiscible material may be heated or warmed to a temperature that causes the unpolymerized mixture (e.g., the fluid matrix material in the unpolymerized material) to polymerize. Once formed, the MOSs may be separated from the immiscible fluid, e.g., the MOSs may be washed to remove the immiscible fluid 611, and placed in a culture media to allow the cells within the MOSs to grow. The MOSs may be cultured for any desired time, or may be cryopreserved and/or assayed immediately. In some embodiments the MOSs may be cultured for a brief period of time (e.g., for between 1-3 days, between 1-4 days, between 1-5 days, between 1-6 days, between 1-7 days, between 1-8 days, between 1-9 days, between 1-10 days, between 1-11 days, between 1-14 days etc.). This may allow the cells derived from the dissociated biopsy tissue to grow and/or divide (e.g., double) for up to five or six passages. After culturing, the cells may be either or both cryopreserved 615 and/or assayed 617. Examples of assays that may be used are also described herein.

In any of these methods and apparatuses described herein, the MOSs may be recovered from the immiscible fluid (e.g., oil) after polymerization. For example, in some embodiments, the MOSs may be recovered by demulsficiation and/or de-emulsification, for example, by forming emulsified droplets and recovering the MOSs after the droplets are formed to remove any oil (and other contaminants). This may allow the cells to grow within the polymerized droplet (the MOS) without being inhibited by the immiscible fluid.

Although the methods and apparatuses described herein illustrate methods of forming the plurality of droplets, and thus the plurality of MOSs, by streaming the unpolymerized mixture into one or more streams of immiscible fluid (such as an oil or other hydrophobic material), in some embodiments the droplets may be formed by other methods that may allow for the size of the droplet to be controlled as described herein. For example in some embodiments the droplets may be formed by printing (e.g., by printing droplets onto a surface). This may reduce or eliminate the need for an additional recovery step of emulsification/de-emulsification. For example, the droplets may be printed onto a surface, such as a flat or shaped surface, and polymerized. In any of these embodiments, the droplets may be dispensed using pressure, sound, charge, etc. In some embodiments, the droplets may be formed using an automatic dispenser (e.g., pipetting device) adapted to release the small amount of the unpolymerized mixture onto a surface, into the air, and/or into a liquid medium (including an immiscible fluid).

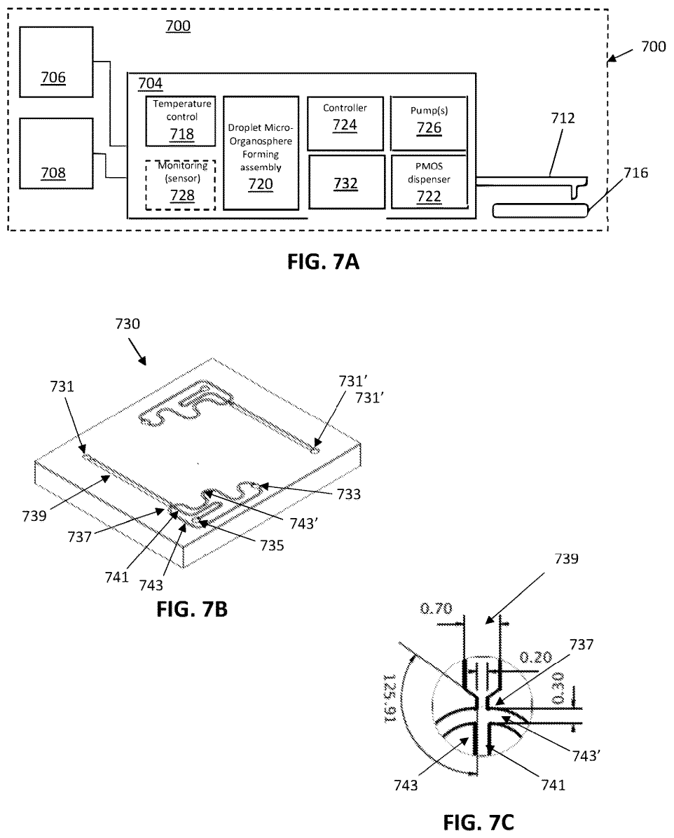

The method for forming the MOSs may be automated, or performed using one or more apparatuses. In particular, the method of forming the MOSs may be performed by an apparatus that allows the selection and/or control of the size of the MOSs (and therefore the density of the number of cells). For example, FIG. 7A illustrates one example of an apparatus 700 for forming MOSs as described.

In FIG. 7A, the apparatus typically includes an input for inputting either the unpolymerized mixture of the dissociated tissue sample and a fluid matrix material (already combined) or may separately receive the dissociated tissue sample, e.g., in a holding solution, and a fluid matrix material. In some embodiments the apparatus includes a holding chamber 706 for holding the unpolymerized mixture, and/or holding chambers (not shown) for holding the dissociated tissue (e.g., biopsy) sample and holding the fluid matrix material. Any or all of these holding chambers may be pressurized to control and/or speed up fluid flow out of the chambers and into the device. The apparatus may either receive the unpolymerized mixture or it may receive the components and mix it. In some embodiments the apparatus may control the concentration of the cells in the unpolymerized mixture and may dilute the mixture (e.g., by adding additional fluid matrix material to achieve a desired density. For example, the apparatus may include a sensor (e.g., an optical reader) for reading the density (e.g., the optical density) of the cells in the unpolymerized mixture (not shown). The sensor may also be coupled to the controller 724, which may automatically or semi-automatically (e.g., by indicating to a user) control the dilution of the cells in the unpolymerized mixture. The apparatuses may also include a port for receiving the unpolymerized mixture. The port may include a valve or may be coupled to a valve and the valve may be controlled by the controller 724 (or a separate controller).

The apparatus 700 may include a chamber 708 and/or port for holding and/or receiving the immiscible fluid. In some embodiments the immiscible fluid may be held in a pressurized chamber so that the flow rate may be controlled. Any of the pressurized chambers may be controlled by the controller 724 which may use one or more pumps 726 to control the pressure and therefore the flow through the apparatus. One or more pressure and/or flow sensors may be included in the system to monitor the flow through the device.

In FIG. 7A, the entire apparatus 700 may be enclosed in a housing 702 or a portion of the apparatus 704 may be enclosed in a housing. In some embodiments the housing may include one or more openings or access portions on the device, e.g., for adding the immiscible fluid and/or the unpolymerized mixture.

As mentioned, any of these apparatuses 700 may also include one or more sensors 728 for monitoring all or key portions of the manufacturing process. In some embodiments, the sensors may include optical sensors, mechanical sensors, voltage and/or resistance (or capacitance, or inductance) sensors, force sensors, etc. These sensors may be used to monitor the ongoing operation of the assembly, including the formation of the MOSs. The apparatus 700 may also include one or more thermal/temperature regulators 718 for controlling the temperatures of either or both the immiscible fluid and/or the unpolymerized mixture (and/or the fluid matrix material).

Any of these apparatuses may also include one or more droplet forming assemblies 720 that may be monitored (e.g., using one or more sensors) as will be illustrated below in FIGS. 7C and 9. The droplet MOS forming assembly may include (or may be coupled with) a dispenser (e.g., a MOS dispenser) 722. The dispenser may dispense, for example, into a multi-well plate 716.

In general, the droplet MOS forming assembly 720 may include one or more microfluidic chips 730 or structures that form and control the streams of the unpolymerized mixture and forms the actual droplets. FIG. 7B illustrates one example of a microfluidic chip for forming MOSs 730. In FIG. 7B, the chip 730 includes a pair of parallel structures for forming MOSs. FIG. 7C illustrates the droplet-forming region of the microfluidic chip for forming MOSs, including an unpolymerized channel outlet 741 that opens (in this example, as a right angle) a “+” junction or region of intersection 737 to the channel outlet 741 and the immiscible fluid outlet(s), 743, 743′. In some embodiments the input from the immiscible fluid channel(s) may be at an angle relative to the angle (and point of intersection) with the unpolymerized material. In FIG. 7C, as in all figures in this description showing dimensions, the dimensions shown are exemplary only, and are not intended to be limiting, unless they otherwise specify.

In FIG. 7A, the microfluidics chip 730 includes an inlet (input port) 733 for the immiscible fluid into the chip (e.g., from the inlet port or storage chamber shown in FIG. 7A). A second inlet port 735 into the chip may be configured to receive the unpolymerized material and transport it down a semi-tortious path to the junction region. Similarly, the inlet port for the immiscible fluid, may be securely coupled to the outlet from the immiscible fluid chamber or inlet, described above.

The inlet port 735 for the unpolymerized material into the chip may be coupled through a delivery pathway 741 connecting the inlet to the junction region (as shown in FIG. 7C). Similarly, the inlet 733 for the immiscible fluid may connect to two (or more) connecting paths 743, 743′ to the junction region 737. A channel leaving the junction region 737 may pass the formed MOSs (in the immiscible fluid) down the channel to an outlet 731 that may connect to a dispenser (not show) for dispensing from the MOSs into one or more chambers, e.g., for culture and/or assaying.

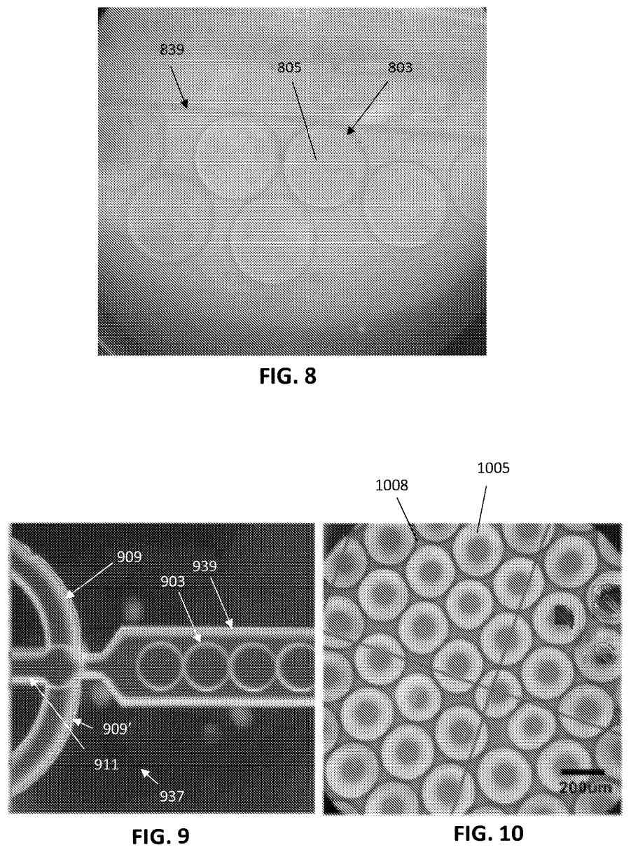

In the example shown in FIGS. 7B and 7C the formed droplets, which may become MOSs once polymerized, may be transmitted down a long, temperature controlled microfluidics environment, prior to being dispensed from the apparatus (not shown). For example FIG. 8 illustrates one example of a channel region 839 (e.g., element 739 in FIG. 7B) that is shown transparent, containing a plurality of MOSs 803 each containing a predetermined number of cell 805.

In FIG. 9, the junction region 937 is shaped as described above, so that the channel carrying the unpolymerized mixture 911 intersects one or more (e.g., two) channels 909 carrying a fluid, such as an oil, that is immiscible with the unpolymerized mixture. As the unpolymerized mixture is pressurized to flow at first rate out of the first channel 911, the flowing immiscible fluid in the intersecting channels, 909, 909′, permit a predefined amount of the unpolymerized mixture to pass before pinching it off to form a droplet 903 that is passed into the outlet channel 939. Thus, in some embodiments, a minced (e.g., dissociated) clinical (e.g., biopsy or resected) sample of tissue, such as <1 mm in diameter, may be is mixed with a temperature-sensitive gel (i.e. MATRIGEL, at 4 degrees C.) to form the unpolymerized mixture. This unpolymerized mixture may be placed into the microfluidic device that may generate droplets (e.g., water-in-oil droplets) that are uniform in volume and material composition. Simultaneously, the dissociated tumor cells may be partitioned into these droplets. The gel in the unpolymerized material may solidify upon heating (e.g., at 37 degrees C.), and the resulting MOSs may be formed. In some embodiments this method may be used to produce over 10,000 (e.g., over 20,000, over 30,000, over 40,000, over 50,000, over 60,000, over 70,000, over 80,000, over 90,000, over 100,000) uniform droplets (MOSs) from the tissue (e.g., biopsy material). These MOSs are compatible with traditional 3D cell culture techniques. FIG. 10 illustrates a plurality of MOSs 1005 formed as described above, suspended in the immiscible material 1008 (e.g., oil).

In some embodiments, chunk preparation can be utilized to improve the number and/or persistence of the immune cells in the MOSs. This can be done, for example, by dissociating patient derived tissues (e.g., tumor tissue) with a collagenase-based enzyme digestion protocol. In some embodiments, chunk preparation includes mincing patient derived tissue and incubating it (e.g., at 37° C.) in a digestion solution (which may be dependent on tissue type) with agitation. Chunks can then be identified in clumps of cells (e.g., 50-100 cells). These chunks can then be filtered into the MOS generator (to avoid clogging). Alternatively, enlarging the diameter of the vessels in the MOS generator can also be done so that chunks are preserved in each MOS.

Utilizing the chunk preparation allows for (i) rapid MOS generation (and consequently rapid testing); (ii) observations of heterogeneity; and (iii) local immune or stromal components are kept in single organoids.

In the exemplary microfluidics chip illustrated above, the junction is shown as a T- or X-junction in which the flow focusing of the microfluidics forms the controllable size of the MOS. In some embodiments, rather than a microfluidics chip, the droplets may be formed by robotic micro-pipetting, e.g., into an immiscible fluid and/or onto a solid or gel substrate. Alternatively in some embodiments the droplets of unpolymerized material may be formed in the requisite dimensions and reproducibility by micro-capillary generation. Other examples of techniques that may alternatively be used for forming the MOSs in the specified size range and reproducibility from the unpolymerized material may include colloid manipulation, e.g., via external forces such as acoustics, magnetics, inertial, electrowetting, or gravitational.

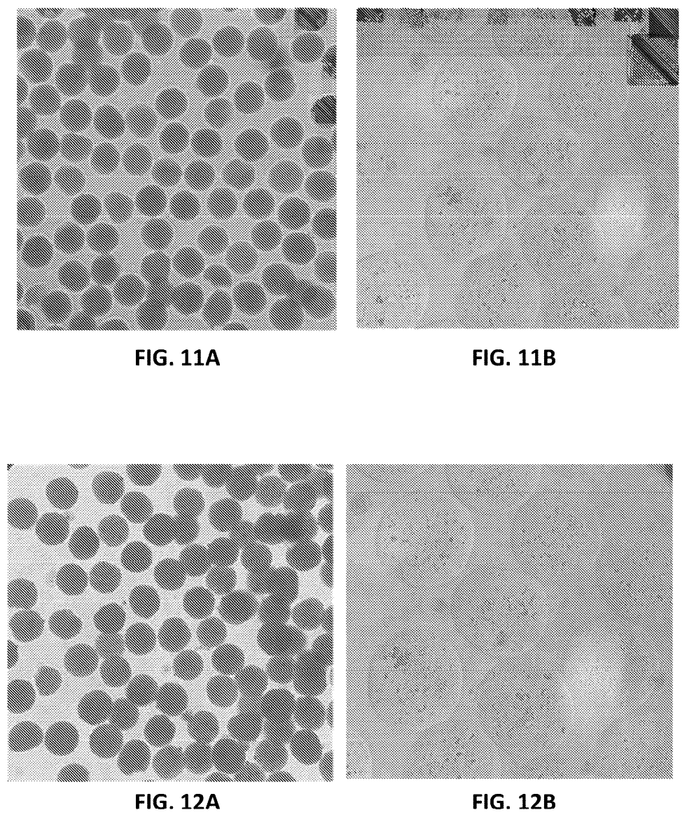

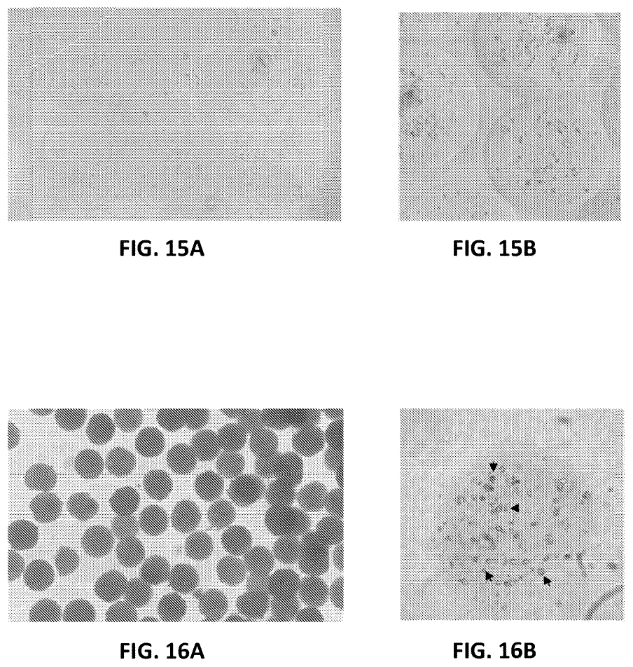

FIGS. 11A and 11B show examples of MOSs in oil formed as described above. The cells within these MOSs, derived from a single biopsy sample, are viable, as seen by vital dye staining, as shown in FIGS. 15A-15B and 16A-16B. For example, FIG. 12A-12B illustrates MOSs having tumor cells (similar to those shown in FIG. 11A-11B) that may be washed to remove the immiscible material (e.g., oil). This immiscible material may be removed relatively quickly after forming the MOS in order to prevent harm to the cells within the MOS.

In some embodiments, membrane-based demulsification, (e.g., using a hydrophobic membrane to remove oil from MOSs) may be utilized to improve persistence of the immune cells in the MOSs.

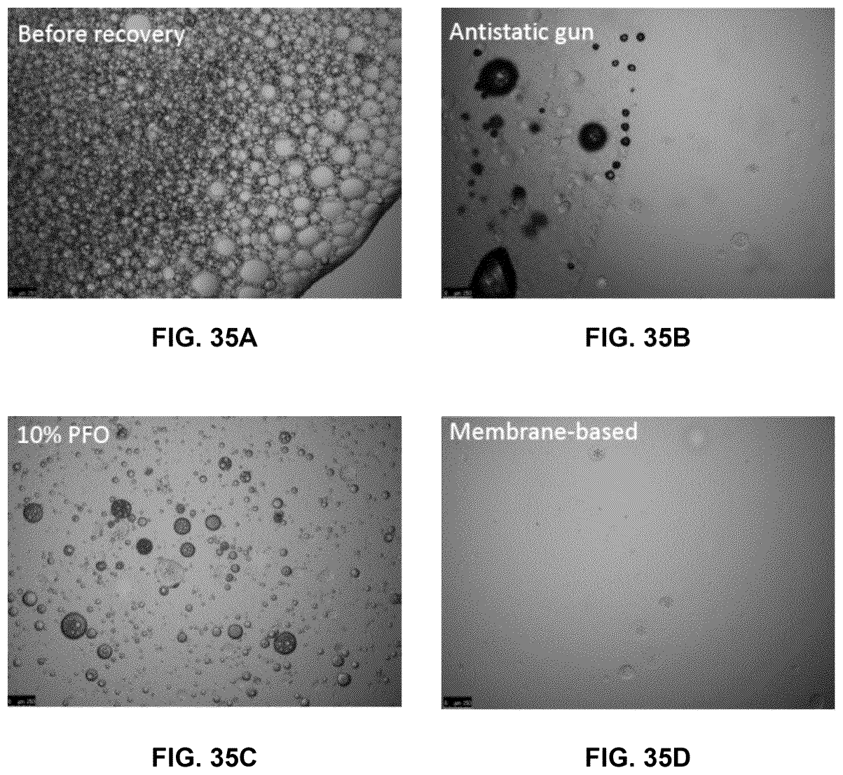

FIG. 35A shows an example of gel droplets in oil. The gel droplets may be used for an assay immediately, cultured, and/or stored (e.g., by cryopreservation). However, it has been found that the viability, particularly in culture, is negatively affected by including oil with the gel droplets. Further, the presence of oil may make it difficult or impossible to accurately assay and/or manipulate the gel droplets. For example, the gel droplets within (or including some) oil may clump or cluster together, preventing isolation and manipulation of individual gel droplets. Thus, it is desirable to remove the oil.

FIGS. 35B and 35C illustrate examples of methods that may be used for removing oil from the gel droplets; these techniques are less thorough and effective, and may in fact be more complicated, than the methods also described herein using a porous hydrophobic surface. For example, FIG. 35B illustrates the gel droplets for which an antistatic gun was used to remove oil. In this example, as can be seen in FIG. 35B, the oil was not completely removed, and further, the resulting gel droplets (shown here by asterisks) are left with oil and debris, possibly resulting from destruction of some gel droplets during the demulsification step. Alternatively, FIG. 35C illustrates the use of a chemical demulsification agent, in this case PFO (10% PFO) used to remove oil from the gel droplets. In this case, the demulsification agent (e.g., PFO) may remove the oil, however, the demulsification agent may also have associated toxicity with respect to the gel droplets. Further, the associated washing/rinsing steps may add additional time and cost to the purification and processing steps. In FIG. 35C, the gel droplets are recovered from the oil phase and resuspended, e.g., into PBS via PFO (perfluoro octanol) and centrifugation. This may separate the immiscible fluid from the gel droplets. Thus, these gel droplets, including tumor based MOSs, can be successfully grown.

FIG. 35D illustrates gel droplets that have been processed to remove the oil using a porous hydrophobic surface (e.g., membrane) as described herein. As can be shown in FIG. 35D, the resulting gel droplets (also indicated by asterisks) generally appear ‘cleaner’ than those produced in FIGS. 35B and 35C with less debris and with little or no residual oil.

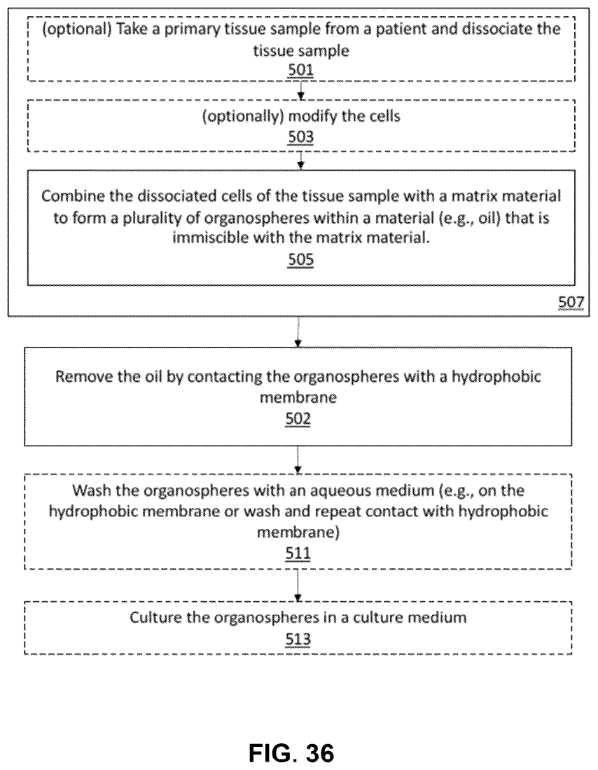

FIG. 36 illustrates one example of a general method of forming/and or processing (including removing oil) gel droplets using a porous hydrophobic surface. In FIG. 36, the method may optionally begin with a primary tissue sample (or other source of cells to be included in the gel droplets); the tissue sample may be dissociated and/or suspended 501. As mentioned above, in some embodiments the cells may be modified 503. The dissociated cells may then be combined with an unpolymerized matrix material 505, and streamed into an oil to form the gel droplets within the oil; the matrix material with the combined dissociated cells (and any additional components) may then be polymerized, as described above, e.g., by increasing the temperature. These steps may generally be part of a step or multiple steps for forming the gel droplets 507, and all or some of these steps may be automated, e.g., by an apparatus.

As shown in FIG. 36, the oil may then be removed using a porous hydrophobic surface (e.g., membrane) 502. In some cases it may be preferable to remove excess oil from the gel droplets first, before exposing to the porous hydrophobic surface, as a rough method to remove the bulk of the oil. For example, the gel droplets may be separated from some of the oil by spinning, followed by removing of the oil layer (e.g., the bottom layer) by pipetting. Alternatively, the oil and gel droplets suspended therein may be directly placed into contact with the porous hydrophobic surface. In any event, the gel droplets may de-emulsified using a porous hydrophobic membrane, as described in greater detail below. This step may remove all or substantially all of the oil (e.g., greater than 99%), and may be quick and easy to perform.

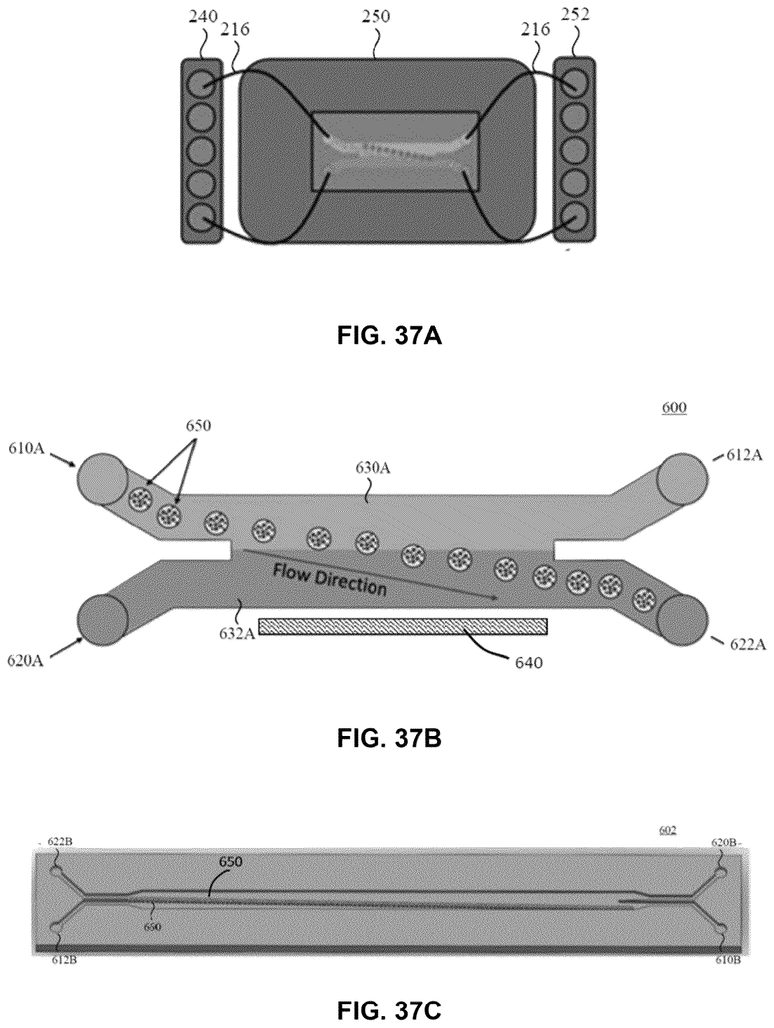

FIG. 37A is a schematic diagram of a demulsifier 250 coupled to a polymerizer 240 and an output 252. For example, the demulsifier 250 may be coupled to an output of the polymerizer 240 and the output 252 may be coupled to an output of the demulsifier 250. In some embodiments, the polymerizer 240 and demulsifier 250 may be temperature regulated at about 37° C.

FIG. 37B is a schematic diagram of a demulsifier 600 based on magnetic separation. The demulsifier 600 may comprise a first inlet 610A (e.g., oil and micro-organosphere inlet), first outlet 612A (e.g., oil and waste outlet), second inlet 620A (e.g., growth media and wash inlet), and second outlet 622A (e.g., growth media and micro-organosphere outlet). The first inlet 610A and the second inlet 620A may be disposed on a first side of the demulsifier 600 and the first outlet 612A and the second outlet 622A may be disposed on a second side of the demulsifier 600 opposite the first side. In some embodiments, a mixture of a first fluid (e.g., oil) and polymerized micro-organospheres 650 may be received in first inlet 610A. A second fluid (e.g., growth media, wash fluid, aqueous solution) may be received in second inlet 620A. The demulsifier 600 may be configured for laminar flow, as shown in FIG. 6A, such that the hydrophobic properties of the aqueous fluid from the second inlet 620A and oil from first inlet 610A do not mix within the demulsifier 600. Instead, a first flow stream 630A (e.g., oil flow stream) and a second flow stream 632A (e.g., aqueous flow stream) flow through the demulsifier 600 in parallel. The demulsifier 600 may further comprise a magnet 640 that serves as a flow separator configured to separate the micro-organospheres 650 that contain magnetic nanoparticles from the first flow stream 630A (e.g., oil flow stream). The magnet 640 may be configured to extend along a length of the demulsifier 600. As the micro-organospheres 650 flow through the demulsifier 600, the magnet 640 may separate the micro-organospheres 650 from the first flow stream 630A (e.g., an oil flow stream) and into the second flow stream 632A (e.g., an aqueous flow stream).

FIG. 37C is a demulsifier 602 that takes advantage of laminar flow properties and small microstructures (e.g., micro-pillars) to filter polymerized droplets from oil into media. The demulsifier 602 may comprise a first inlet 610B (e.g., oil and micro-organosphere inlet), first outlet 612B (e.g., oil and waste outlet), second inlet 620B (e.g., growth media and wash inlet), and second outlet 622B (e.g., growth media and micro-organosphere outlet). The first inlet 610B and the second inlet 620B may be disposed on a first side of the demulsifier 602 and the first outlet 612B and the second outlet 622B may be disposed on a second side of the demulsifier 602 opposite the first side. In some embodiments, a mixture of a first fluid (e.g., oil) and polymerized micro-organospheres 650 may be received in first inlet 610B. A second fluid (e.g., growth media, wash fluid, aqueous solution) may be received in second inlet 620B. The demulsifier 602 may be configured for laminar flow, as shown in FIG. 6B, such that the hydrophobic properties of the aqueous fluid from second inlet 620B and oil from first inlet 610B do not mix within the demulsifier 602. Instead, a first flow stream (e.g., an oil flow stream (not shown)) and a second flow stream (e.g., an aqueous flow stream (not shown)) flow through the demulsifier 602 in parallel. In some embodiments, the demulsifier 602 may comprise a flow separator 660 (e.g., a set of micro-pillars) configured to separate micro-organospheres 650 from the first flow stream (e.g., oil flow stream). The flow separator 660 may be configured to extend along a length of the demulsifier 602. As the micro-organospheres 650 flow through the demulsifier 602, the micro-pillars of the flow separator 640 may separate the micro-organospheres 650 from the first flow stream (e.g., oil flow stream) and into the second flow stream (e.g., aqueous flow stream). In some embodiments, the micro-pillars can be positioned at an angle of about one degree from the first flow stream (e.g., oil flow stream) into the second flow stream (e.g., aqueous flow stream), thereby forcing the micro-organospheres 650 from the first flow stream into the second flow stream while allowing each flow stream to remain flowing in parallel. The spacing of the micro-pillars is such that the micro organospheres 650 are unable to pass through the micro-pillars and the spacing can be varied in fabrication depending on the expected droplet size.

As described in the examples below, MOSs described herein provide a good model for the effectiveness of various drug formations. A variety of drugs can be used and interacted with MOSs. Exemplary drugs include (but are not limited to) MAPK inhibitors (e.g., Vemurafenib, Dabrafenib, PLX8349, Cobimetinib, Trametinib, Selumetinib, and BVD-523), checkpoint inhibitors (e.g., T-cell targeted immunomodulators, Pembrolizumab, Avelumab, Durvalumab, Ipilimumab, TSR-022, MGB453, BMS-986016, and LAG525), other immunomodulators (e.g., anti-CD47 antibodies, and ADCC therapies), apoptosis inhibitors (e.g., ABT-737, WEHI-539, ABT-199) potential contributing pathways (e.g., Afuresetib, Idasanutlin, and Infliximab), chemotherapy agents (e.g., Cytarabine), cell therapy, cancer vaccine, oncolytic viruses, and bi-specific antibodies.

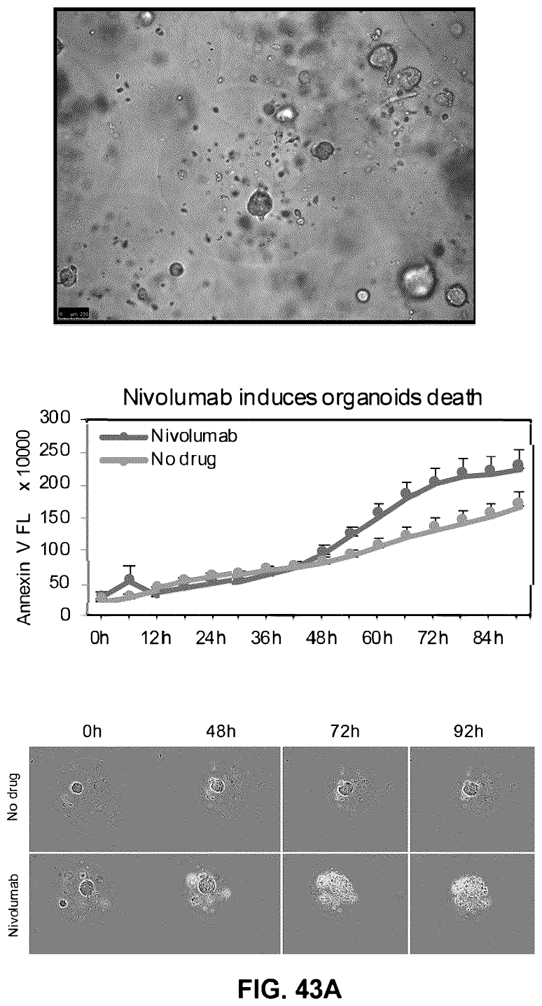

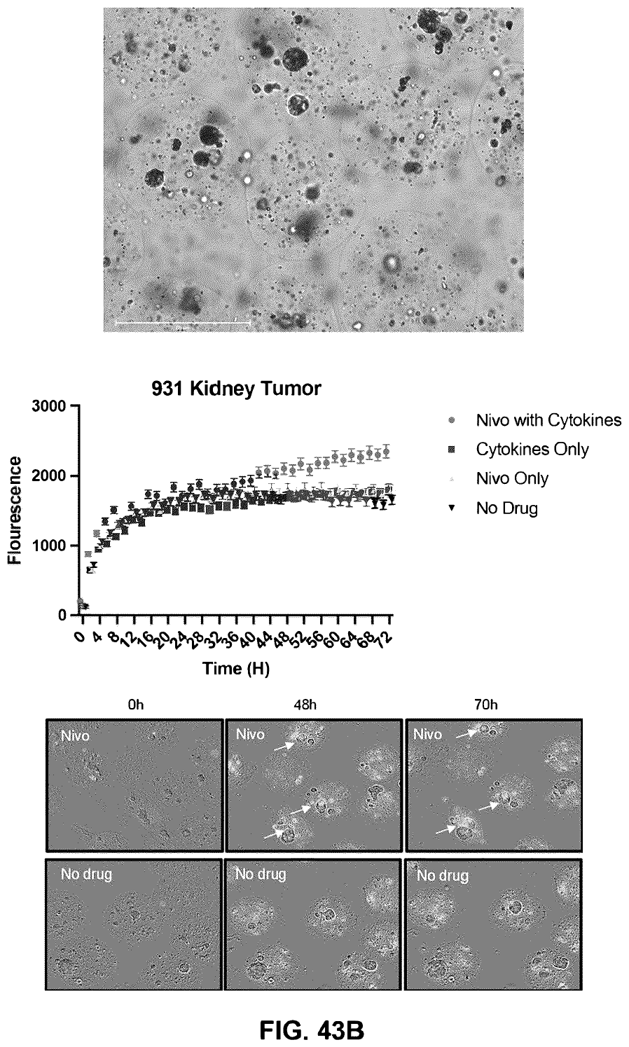

FIGS. 43A and 43B shows the efficacy of Nivolumab on MOSs derived from pulmonary and renal tumor biopsies. Assays of the MOSs for these pulmonary and renal tumor biopsies were prepared with an Annexin V marker to indicate cellular apoptosis. As can be seen in FIGS. 43A and 43B, the MOSs display a good response to Nivolumab (in the form of tumor cell apoptosis). If this test were run on, for example, traditional bulk organoids, the results would not be meaningful (due to the lack of patient immune cells) leading to uncertainty in the best course of action for patient treatment.

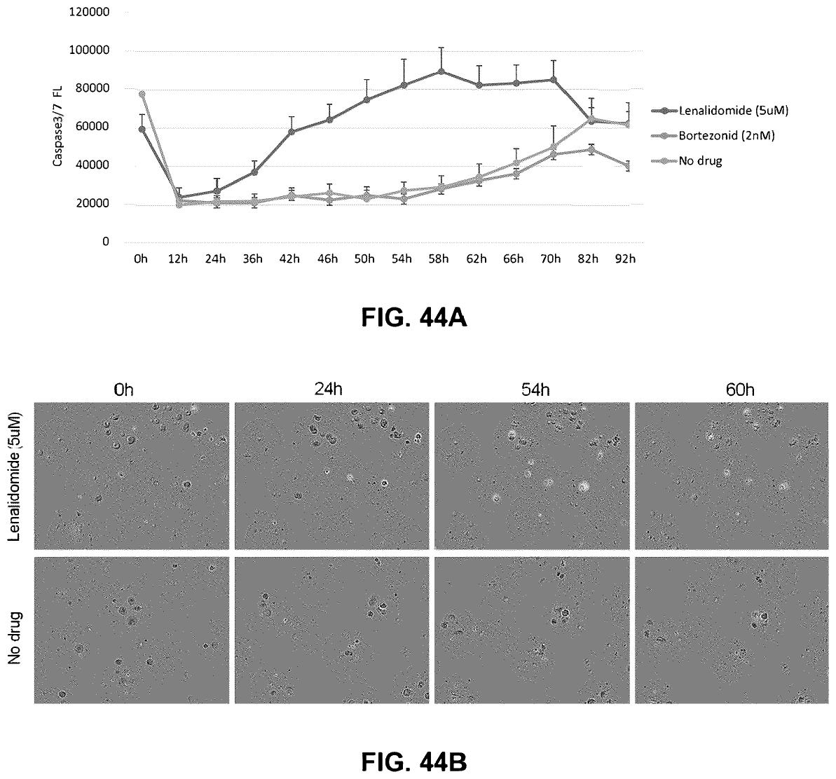

FIGS. 44A and 44B show the efficacy of Lenalidomide and Bortezoid on MOSs derived from multiple myeloma (MM) bone marrow biopsies. MM MOSs (on day 11) were treated with Lenalidomine (5 uM) or Bortezoid (2 nM). Caspase 3/7 green dye was added in the assay to monitor apoptosis. Incucyte images were taken every 2 hours for 4 days. As can be seen in FIGS. 44A and 44B, the MOSs display a good response to Lenalidomide (in the form of tumor cell apoptosis) but not to Bortezoid. This result would give a healthcare provider confidence that Lenalidomide is a better choice than Bortezoid for treating this particular MM patient. If this test were run on, for example, traditional bulk organoids, the results would not be reliable (due to the lack of patient immune cells) leading to uncertainty in the best course of action for patient treatment.

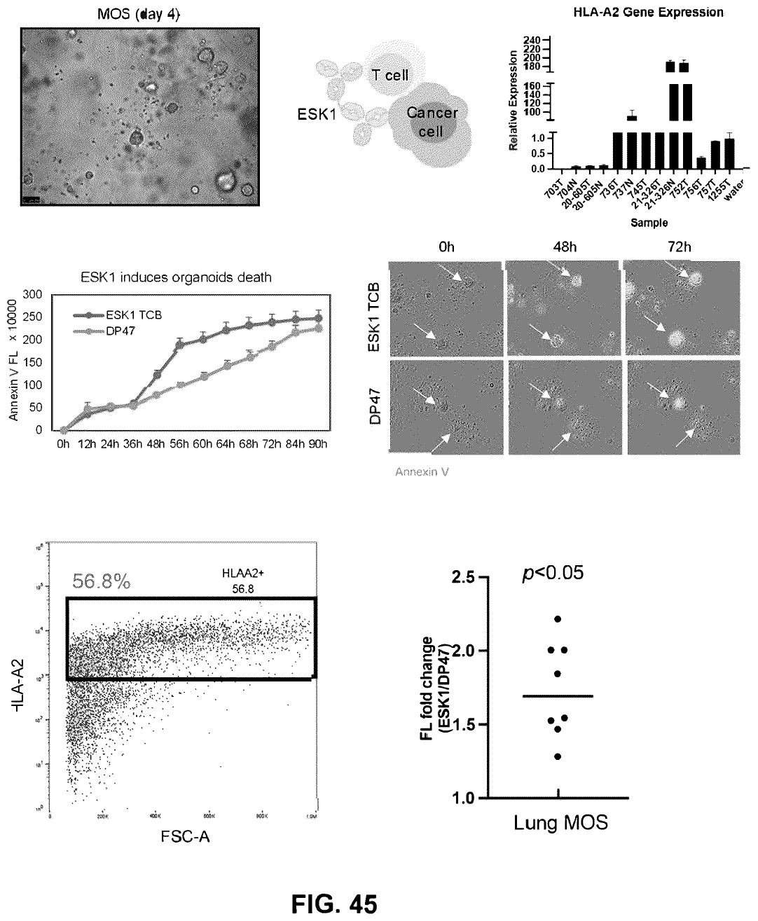

FIG. 45 shows the efficacy of ESK1 (a T-cell receptor-mimic antibody) on MOSs derived from a pulmonary Biopsy. Assays of the MOSs for this pulmonary tumor biopsy were prepared with an Annexin V marker to indicate cellular apoptosis. As can be seen in FIG. 45, the MOSs display a good response to ESK1 (in the form of tumor cell apoptosis). If this test were run on, for example, traditional bulk organoids, the results would be less accurate (because ESK1 would not be able to reach its target) leading to uncertainty in the best course of action for patient treatment.

As described in the examples below, MOSs will readily uptake infused immune cells to provide a good model for the effectiveness of various immune cell therapies.

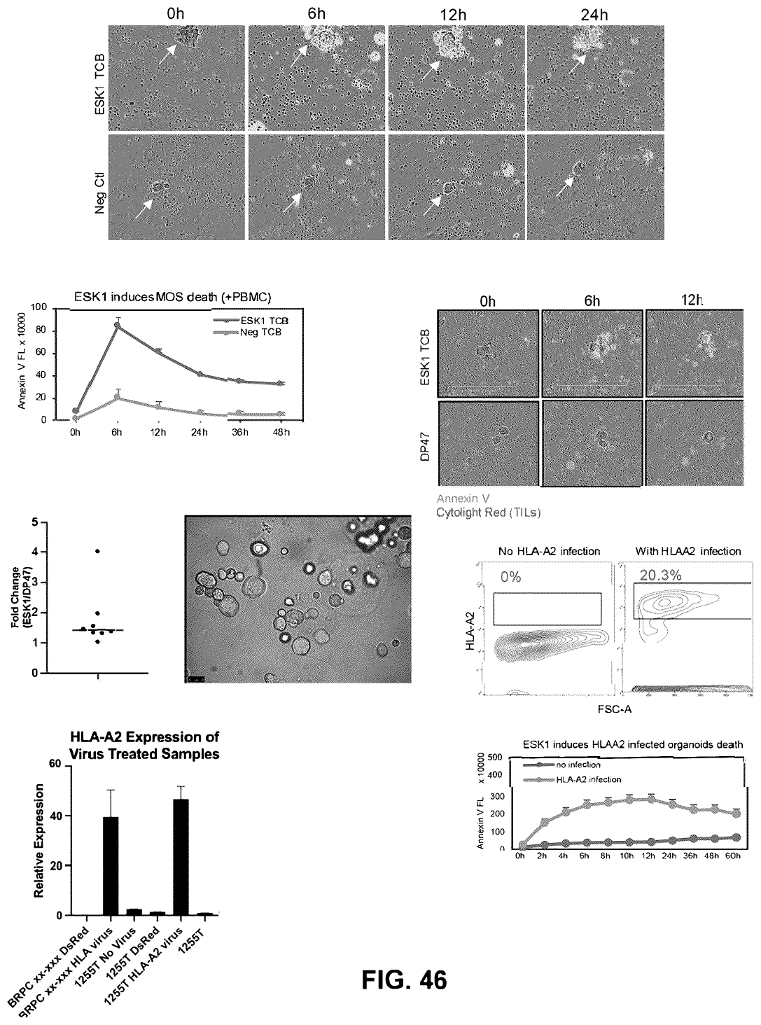

FIG. 46 shows the efficacy of ESK1 in combination with peripheral blood mononuclear cells (PBMC) on MOSs derived from a pulmonary tumor biopsy. Assays of the MOSs for this pulmonary tumor biopsy were prepared with an Annexin V marker to indicate cellular apoptosis and Cytolight Red dye to indicate tumor infiltrating lymphocytes (TIIs). As can be seen in FIG. 46, the MOSs display a good response to ESK1 with PBMC (in the form of tumor cell apoptosis). If this test were run on, for example, traditional bulk organoids, the results would be less accurate (due to the inability of PBMC to penetrate the traditional bulk organoids) leading to uncertainty in the best course of action for patient treatment.

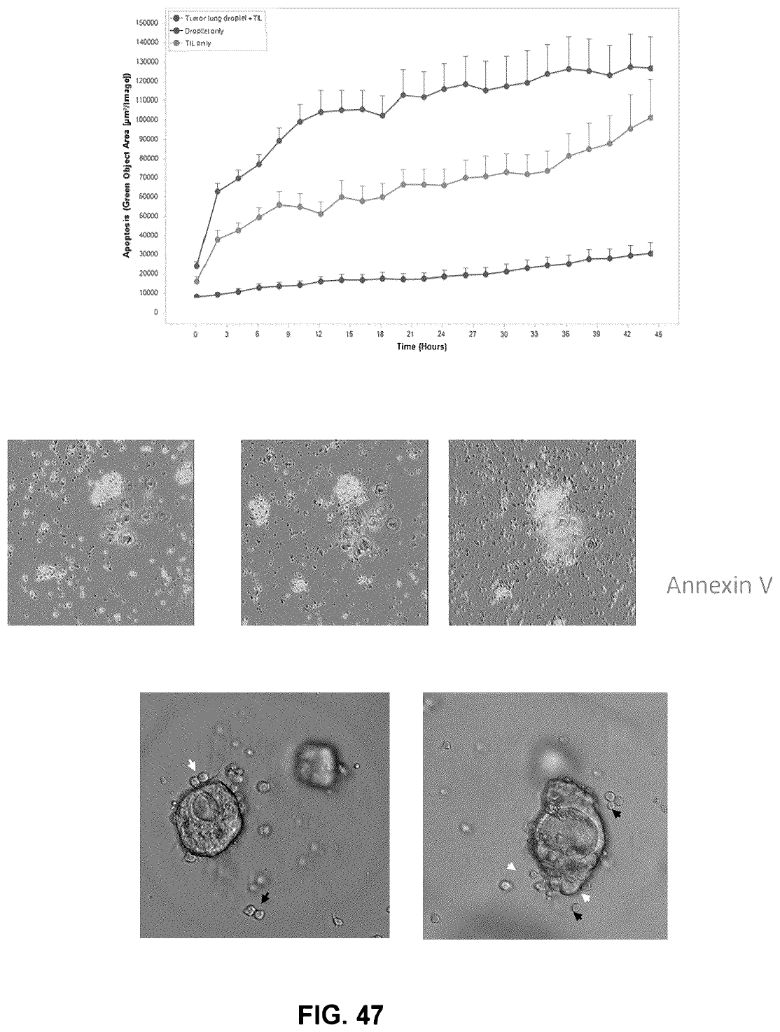

FIG. 47 shows the efficacy of TIIs on MOSs derived from a pulmonary tumor biopsy. Assays of the MOSs for this pulmonary tumor biopsy were prepared with an Annexin V marker to indicate cellular apoptosis. As can be seen in FIG. 28, the MOSs display a good response to TIIs (in the form of tumor cell apoptosis). This is an indication that TIIs can penetrate MOS to kill tumor cells.

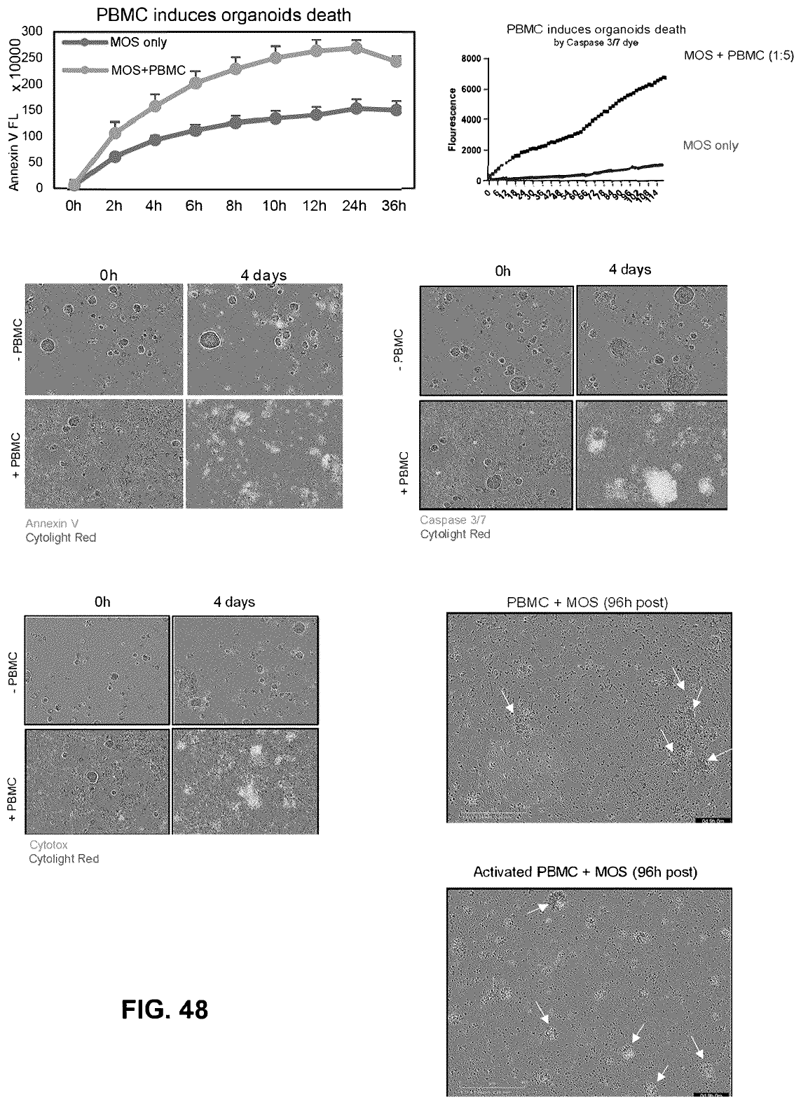

FIG. 48 shows the efficacy of PBMC on MOSs derived from a pulmonary tumor biopsy. Assays of the MOSs for this pulmonary tumor biopsy were prepared with an Annexin V marker to indicate cellular apoptosis and Cytolight Red dye to indicate tumor infiltrating lymphocytes (TIIs). As can be seen in FIG. 48, the MOSs display a good response to PBMC (in the form of tumor cell apoptosis).

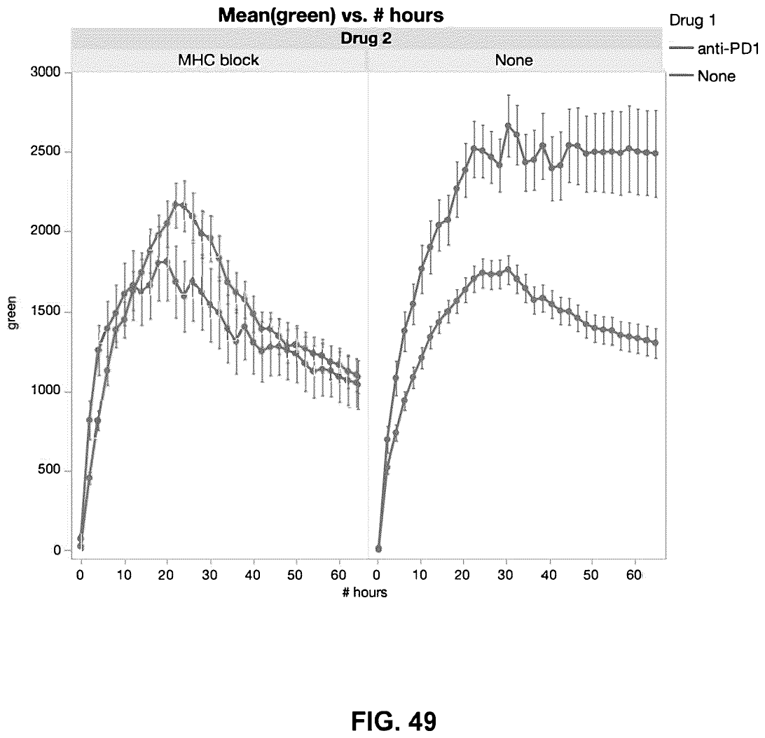

FIG. 49 shows the efficacy of anti-PD1 (e.g., Nivolumab) in combination with TIIs on MOSs. As can be seen in FIG. 49, the MOSs display a good response to anti-PD1 with TIIs (in the form of tumor cell apoptosis). If this test were run on, for example, traditional bulk organoids, the results would be less accurate (due to the inability of TIIs and nivolumab to penetrate the traditional bulk organoids) leading to uncertainty in the best course of action for patient treatment.

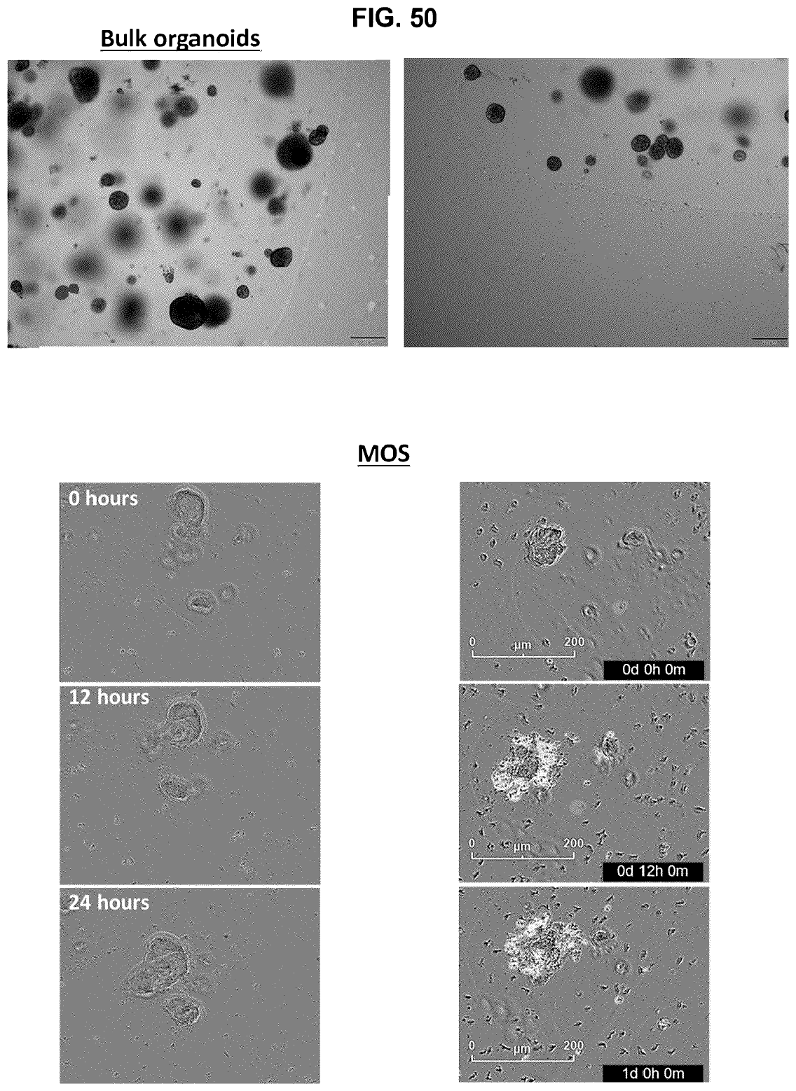

FIG. 50 shows the difference in the ability of infused patient derived T cells to penetrate bulk organoids vs. MOSs. As mentioned above, immune cells have difficulty penetrating traditional bulk organoids. This can be seen directly in FIG. 50 where T-cells are able to penetrate into the MOS and are unable to penetrate the Bulk Organoids.

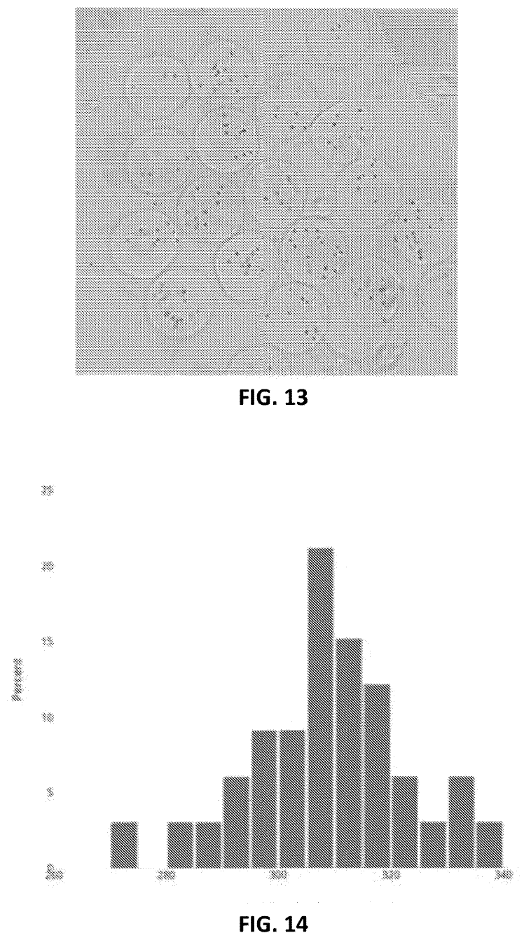

In some embodiments, the gel droplets are recovered from the oil phase and resuspended, e.g., into PBS via PFO (perfluoro octanol) and centrifugation. This may separate the immiscible fluid from the MOSs. Thus, these MOSs, including tumor-based MOSs, can be successfully grown, as shown in FIGS. 1A-1C, 2A-2C, 3A-3C and 4A-4E, above, and in FIG. 13. This is an important improvement, as drug screening has to be performed on viable and growing primary tumor cells that retain their properties from patient tumors to predict patient outcomes. The high number and uniformity of these MOSs makes screening both possible and reliable, as will be described below.

In any of the microfluidic chips or devices described herein, the channels may be coated. For example, the channel of the microfluidic device may be coated with a hydrophobic material.

In general, the MOSs described herein are highly uniform in diameter, and may have a very low size, e.g., diameter, variance. This is illustrated, for example, in FIG. 14, showing a distribution of one example of droplet diameter sizes.

As mentioned, FIGS. 15A-15B shows MOSs formed as described herein; in FIG. 16A-16B, these MOSs have been stained with Trypan blue (arrowheads), showing that they are alive. The MOSs formed as droplets in this manner may contain growth-factors and matrix to mimic the biological environment from which the tissue arose. Patient samples (e.g., biopsy samples) may be formed into MOSs (including hundreds, thousands, or tens of thousands of MOSs) within a few hours of acquiring the tissue. The MOSs may have as few as 1 or between 4-6 cells (e.g., cancer cells when sampling a tumor) per MOS or as many as hundreds of cells. These methods have been shown to work for virtually all types of cancer and non-cancer tissues tested to date (n=32), including colon, esophagus, melanoma, uterus, sarcoma, renal, liver, ovary, lung, diaphragm, omentum, mediastinal lung, and breast cancer tissues. The MOSs may be cultured for any desired period of time, and typically show proliferation and growth in as few as 3-4 days. They may be maintained and passaged for months. As will be described in greater detail below, they may be used to screen thousands of drug compositions within as few as 4-6 days from taking the tissue (e.g., biopsy).

The MOSs described herein may, at any point after they are formed, be banked, e.g., by cryopreserving them. Tumor MOSs may be collected from many different patients and may be used individually or collectively to screen multiple drug formulations to determine toxicity and/or efficacy. Non-tumorous cells (healthy tissue) may be biopsied, banded and/or screened in parallel. Thus, these methods and apparatuses may allow for high throughput screening. In some embodiments, the MOSs may be formed and allowed to passage twice (e.g., two doublings), and cryopreserved. As mentioned, normal healthy tissue may be used to form these same MOSs to generate hundreds, thousands, or tens of thousands of MOSs that may be used for assaying drug effects, drug response, biomarkers, proteoimic signals, genomic signals, etc.

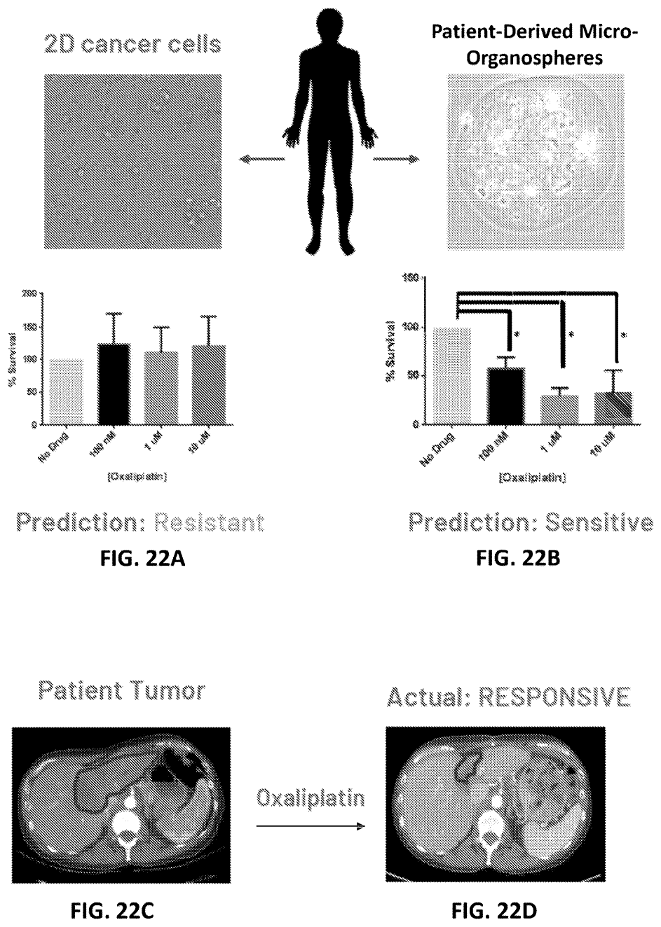

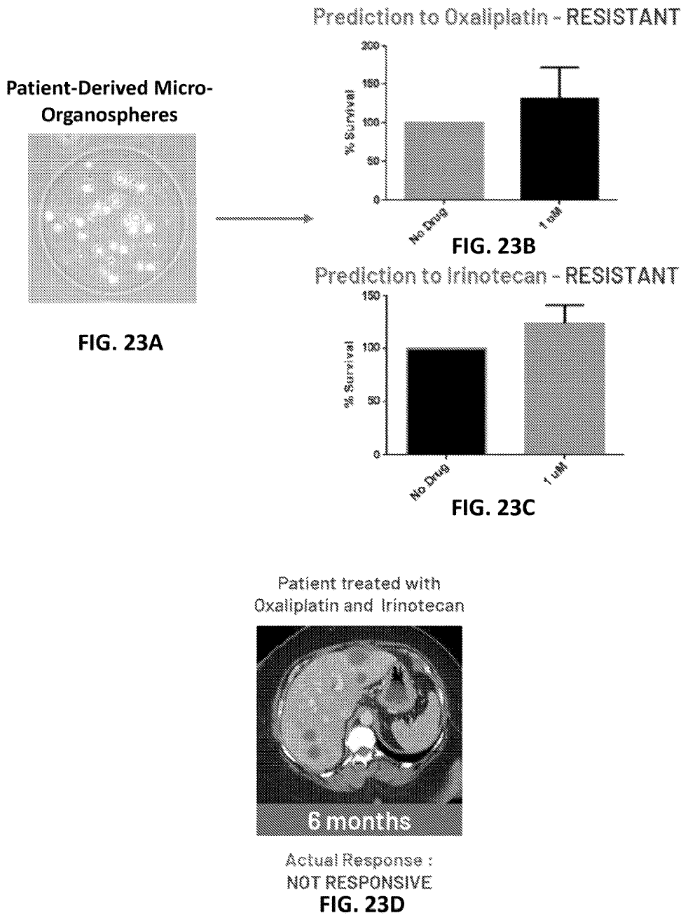

It is of particular significance that these MOSs survive in a biologically significant manner, allowing them to provide clinically and physiologically relevant data, particularly with respect to drug response, as will be described in FIGS. 22A-22D and 23A-23D. In particular, the MOSs described herein permit tissue extract/biopsy originated cells to grow exceptionally well and provide more representative data, especially as compared to organoids or spheroids. Without being bound by a particular theory, this may be because the cells may have a more constrained cell density in the MOSs, permitting cells to communicate without inhibiting each other while sharing signals. The MOSs also have a very large surface to volume ratio, more readily permitting transmission of growth factors and other signals to penetrate into the MOSs (e.g., the MOSs are less diffusion limited).

### Assays

The MOSs described herein may be used in a variety of different assays, and in particular may be used to determine drug formulation effects, including toxicity, on normal and/or abnormal (e.g., cancerous) tissue. For example, drug screening may include applying MOSs into all or some wells of multi-well (e.g., a 96-well) plate. Alternatively custom plates may be used (e.g., a 10,000 micro-well array may be formed of a 100×100 wells). The MOSs (e.g., gel droplets) may be applied into, or in some embodiments onto the multiple microwell arrays and incubated with culture medium. The MOSs may be cultured over the course of 3-5 days. In some embodiments, on day 5, the wells (e.g., micro-reactors) may then be dosed with drug compounds, e.g., based on a set of FDA-approved anticancer drugs, to examine the effects of the drug panel. For example, the drugs texted may be based on the National Cancer Institute (Division of Cancer Treatment and Diagnosis) screen, consisting of 147 agents intended to enable cancer research, drug discovery and combination drug studies. On Day 7, the MOSs may be imaged via standard fluorescent microscopy and ranked based on drug response.

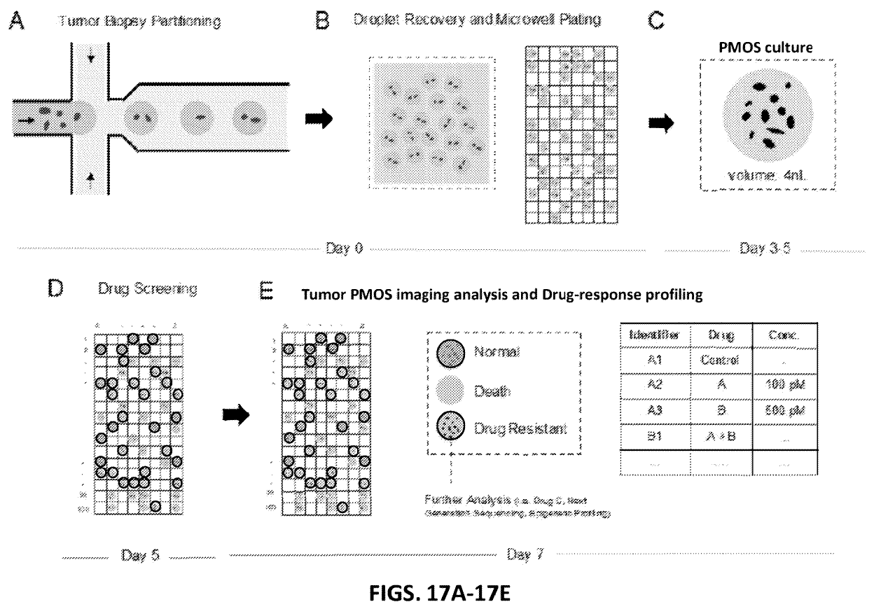

An example of this assaying technique is shown in FIGS. 17A-17E.

In this example, the screening assay may be automated. This may enable repeatable and automated workflow, which may increase the number of drugs screened from a few to hundreds. FIGS. 17A-17E illustrate one example of this workflow. In FIG. 17A, a tumor biopsy is taken and a plurality (e.g., >10,000) MOSs are formed as described above (in FIG. 17A the junction region forming the MOSs is illustrated). Thereafter, the MOSs may be recovered and washed (e.g., to remove the immiscible (e.g., oil) material in which they were formed). The MOSs may then be plated into one or more microwell plates. As shown in FIG. 17C, the MOSs may be cultured for one or more generations (e.g., one or more passages). This is shown occurring from day 0 to days 3, 4 or 5. Thereafter, the MOSs may be screened, as shown in FIG. 17D, e.g., by applying drugs to a subset of the replicant wells. Thereafter, as shown in FIG. 17E, on day 7, the cells in the MOSs may be imaged and/or automatically or manually scored to identify drug effects (e.g., drug screening and growth profiling).

The workflow shown in FIGS. 17A-17E may enable an integrated device to be used for growing, dosing and/or reviewing the MOSs. In one exemplary device, freshly biopsied or resected patient tumor samples may be disassociated and seeded into gel with regents to form the MOSs (as described above). A portion of the MOSs formed may be cryopreserved. The rest may be recovered and incubated until seeded into microwell plates for drug testing or screening as just described. Growth and viability assays may be performed on the MOSs, which may be imaged and tracked. Their response to drug treatments, such as IC-50, cytotoxicity, and growth curves, may be measured to identify effective therapeutics against the patient's tumor.

The methods and apparatuses described herein have numerous advantages, including reproducibility. The sample preparation process may be automated by the microfluidic sample partitioning which may reduce the need for specialized personnel for diagnostic testing and manual pipetting. This may be particularly helpful in a clinical setting. Moreover, this may enable uniformity among signal droplets, increasing assay sensitivity. In addition, these assays may minimize the time required to generate MOSs. Based on preliminary data, these methods may be able to generate a library of over 100,000 MATRIGEL-tumor droplets (MOSs) in less than about 15 minutes. These methods are also highly scalable, and can be multiplexed to run multiple patient biopsies in parallel.

Finally, these methods are flexible and compatible with other techniques. As a research tool, droplet-based microfluidics is generally compatible with a wide range of hydrogel materials such as agarose, alginate, PEG, and hyaluronic acid. As such, the starting gel composition can easily be modified to accompany and encourage MOS growth. Moreover, the droplet-size can be adjusted by modifying the size of our microfluidic device. Together, these allow a large selection of gel material composition and micro-reactor sizes.

The miniaturized assays described here, e.g., using the MOSs, may maximize the patient tumor biopsy, enabling more drug compounds to be screened. For example, a 600 uL tumor sample can be partitioned into ˜143,000 individual micro-reactors that are ˜4 nL in volume. By maximizing the tissue sample, multiple experimental replicates may be examined, increasing statistical power. These techniques may allow the inspection of intra-tumor heterogeneity, drug perturbation and identify rare cellular events, such as drug resistance. The MOSs may generally be compatible with downstream assays including single cell RNA transcriptome analysis and epigenetic profiling. In addition, by maximizing the tissue (e.g., biopsy) sample efficiency as provided by the MOSs, a portion of the MOSs may be banked (e.g., by cryopreservation for biobanking) for future novel drug assays and/or for confirmation analysis, including genetic screening.

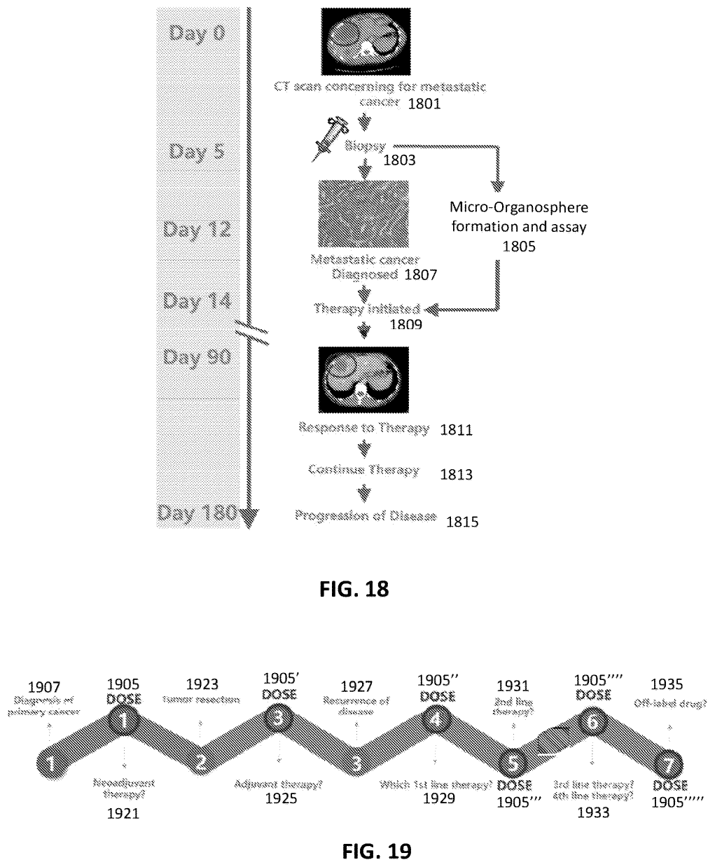

For example, FIGS. 18 and 19 illustrate therapeutic methods that use the methods and apparatuses, including the MOSs, described herein. For precision and personalized medicine, these methods and apparatuses can be used as a clinical indicator for appropriate drug selection to improve clinical outcome and drug response. As one embodiment, a patient diagnosed with metastatic cancer will take a biopsy for histopathology and for screening of a plurality of MOSs formed from a biopsy as described herein. Within 7˜10 days, the screening may be performed from the biopsy to identify the most effective standard-of-care therapy so the patient can start treatment around 14 days.

An example of this is illustrated in FIG. 18. In this example, the tumor may be identified at day 0 (e.g., by CT scan) 1801, and a biopsy taken 1805 at day 5, and on the same day hundreds, thousands, or tens of thousands of MOSs can be formed and cultured for 1-5 days and screened 1805 to identify one or more drug compositions that can be used. This same step (forming the MOSs and screening) may be used to guide precision medicine at multiple clinical decision points throughout disease progression. In this example, therapy using the identified one or more drug compositions may be started on day 14 (1809), and the patient may later be monitored during the course of treatment (e.g., a follow-up CT scan on about day 90) to confirm that the tumor is responding to the treatment 1811. If so, the therapy may be continued 1813 and the ongoing progress monitored 1815.

As mentioned, the use of MOSs to assay may be repeated at multiple point throughout treatment and during the course of the treatment. This is illustrated in FIG. 19. For example, when a patient is first diagnosed 1907 with a resectable primary tumor, this technique (e.g., generation of MOSs and screening 1905) can be used to determine the most effective neoadjuvant therapy 1921. Thus, a biopsy may be taken and hundreds, thousands, or tens of thousands of MOSs may be formed and screened with a panel of potential drug compositions. Once the primary tumor is resected 1923, this technique 1905′ may indicate whether and which adjuvant therapy should be chosen 1925. If recurrence or metastasis happens after the surgical removal of the primary tumor 1927, the same technique (e.g., generating and screening MOSs from a fresh biopsy 1905″, 1905′″, 195″″) can be used to guide standard-of-care therapy, including 1st 1929, 2nd 1931, and 3rd line 1933 therapies. If the patient eventually becomes tolerant or resistant to all standard-of-care therapy, this technique 1905′″″ can be performed to identify off-label drugs to treat resistant tumors 1935. This technique can also be used as companion diagnostics to identify patients for a specific treatment. Lastly, the technique can be used to derive and preserve patient-derived MOSs to establish an Organosphere-based living cancer bank for screening, genomic profiling, new drug discovery, drug testing and clinical trial design.

Because these techniques, and the generation of a huge number of MOSs may be done with relatively low-invasiveness (e.g., by resection or biopsy), to provide reasonably fast results from the screening, these methods may be easily adapted for standard of care. For example, the volume of cellular material from the tissue (e.g., biopsy) input is quite small, and may be dissociated into a volume of, e.g., between 10 μL to 5 ml.

In general, the use of the MOSs described herein for screening may be automated or manually performed. Virtually any screening technique may be used, including imaging by one or more of: confocal microscopy, fluorescent microscopy, liquid lens, holography, sonar, bright and dark field imaging, laser, planar laser sheet, including high-throughput embodiments of image-based analysis methods (e.g., using computer vision, and/or supervised or unsupervised model, e.g., CNN). Downstream screening may include sampling the culture media and/or performing genetic or protein screening (e.g., scRNA-seq, ATAC-seq, proteomics, etc.) on cells from the MOSs.

### EXAMPLES

### Example 1

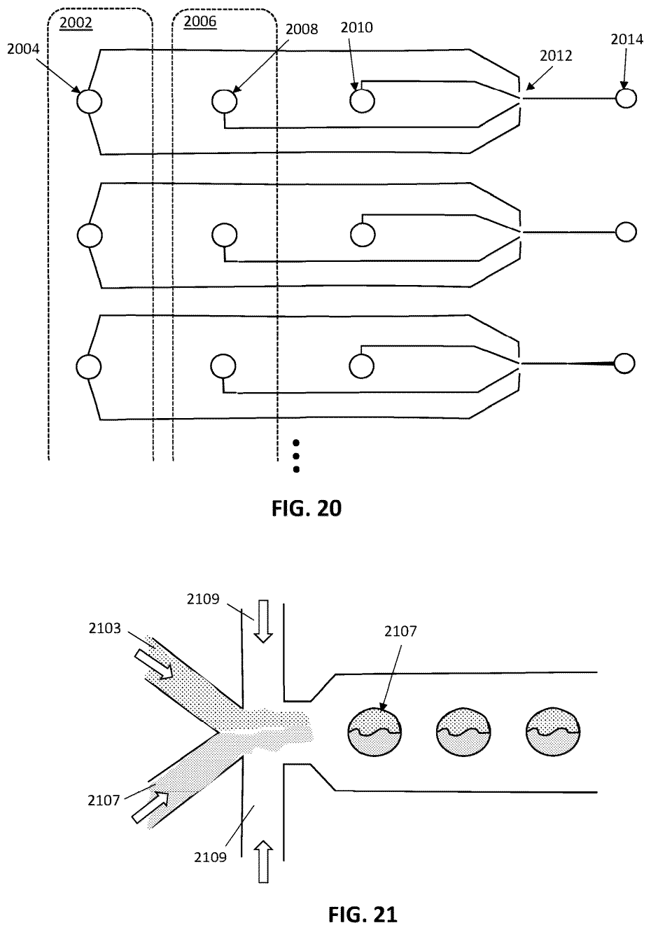

FIGS. 20 and 21 illustrate another example of an apparatus for forming a plurality of MOSs as described herein. In FIG. 20, the apparatus may include a plurality of MOS forming junctions, in which the immiscible material (e.g., oil) 2002 may be added to a reservoir and/or port 2004 in the device. Similarly, the unpolymerized material 2006 (in this example, including the dissociated biopsy cells and the fluid matrix material) may be added to the reservoir or port 2008 in the apparatus. In some embodiments a second or additional material (e.g., a biologically active agent) may be added via a third set of ports 2010. These components may be combined at a junction (similar to that described above) forming a droplet in the immiscible material that may be polymerized into the MOSs. In FIG. 20, three (or more) parallel junctions with corresponding inputs and output are shown.

FIG. 21 illustrates the method of forming the MOSs using an apparatus as shown in FIG. 20. In this embodiment the resulting MOSs include both the target (e.g., tumor) biopsy cells and also one or more additional biologically active agents that are combined to form the MOS. For example, a first channel 2103 may include the unpolymerized material (including the dissociated biopsy cells and the matrix material), a second channel 2107 includes an additional active biological material, and a pair of intersecting channels 2109, 2109′ carrying the immiscible material (e.g., oil) converge at the junction to form the size-controlled droplets that are polymerized to form the MOSs 2107.

In this example the additional active biological material may be, e.g.,

freezing medium (e.g., to aid in banking the MOSs), and/or co-cultures with additional cells (e.g., immune cells, stromal cells, endothelial cells, etc.), additional supportive network molecules (e.g., ECM, collagen, enzymes, glycoproteins, biomimetic scaffolds, etc.), additional growth factors, and/or drug compounds.

### Example 2: Screening Results

As mentioned above, the MOSs and methods of using them to screen for drug compositions may be used to accurately predict the response of a patient tumor to one or more drug therapies. In some cases, the use of MOSs may provide accurate results where traditional cultured drug screening does not accurately predict drug response. For example, in FIG. 22A-22D, the MOSs, but not a cell line, was able to correlate with patient response. In FIG. 22A, a traditional cell line dosed with drugs (e.g., Oxalipalitin) was examined; the cell line showed no effect, predicting that the tumor would be resistant to the drug at all dose ranges examined.

For comparison a plurality of MOSs were generated from a patient biopsy, as shown in FIG. 22B. In this example, the MOSs showed significant decreases in cell survival from the tumor MOSs, predicting drug sensitivity. In fact, when treated with the drug, the tumor responded to treatment, as shown in FIGS. 22C (before treatment) and 22D (post treatment).

### Example 3: Correlation Between MOSs and Patient Response

In a similar set of experiments, MOSs were generated from biopsy material (FIG. 23A), and a drug effect screen was performed using the resulting MOSs. FIG. 23B shows the effect of a first drug (Oxalipalatin) on these MOSs, showing no change in the percent survival of the MOSs in the presence of the drug, predicting drug resistance. Similarly, treatment with a second drug, Irinotecan, showed a lack of effect on the MOSs, predicting drug resistance, as shown in FIG. 23C. The patient was treatment with both Oxalipalatin and Irinotecan, and, after 6 months of treatment, showed no response. Thus, the MOSs correlated strongly with patient response to the standard of care drugs. In this case, the patient endured six months of side effects and toxicities that may have been avoided by the predicted response from the MOSs, indicating (within 7-10 days from the biopsy) that the tumor would not respond to these drugs.

### Example 4: Multi-Drug Screening

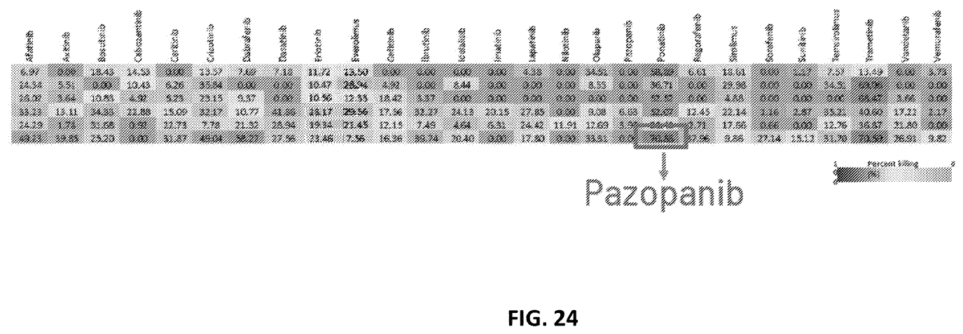

FIG. 24 shows an example of a panel of drug (e.g., chemotherapeutic agents) that may be generated using a Patient Derived plurality of MOSs as described herein. In this example, a drug screen using the Patient-Derived MOSs was run by dosing a plurality of replicates for each of a plurality of (27) drugs. A single tumor biopsy was used to generate a plurality of MOSs in large quantities extremely fast (e.g., within less than two weeks) and these MOSs were tested against the panel of drug formulations (e.g., 27 formulations are shown). This testing was done in parallel and could be automatically quantified (e.g., by optical detection and quantification). In this example, the drug showing the largest toxicity was Pazopanib for this particular tumor.

Combinations of drugs as well as different drug concentrations may be examined in parallel. As hundreds, thousands, or tens of thousands of MOSs may be generated from the same tumor biopsy, array testing of this sort is made practical by the methods and apparatuses described herein.

### Example 5: Biopsy Sample Preparation

Materials: an apparatus for forming the MOSs, as described above, including a droplet microfluidic chip (200 um); Bio-rad Droplet Generation Oil for EvaGreen (catalog #186-4006), 3-5 mL per run, Perfluoro octanol (PFO), Sigma, 10% Perfluoro octanol (PFO) in Novec HFE 7500, PBS, Cell culture media (i.e. RPMI w/ 10% FBS and 1% PenStrep), 70 um or 100 um filters, 50 ml conical, Petri dish.

Biopsy sample dissociation: using a biopsy sample (human/animal) to generate a dissociated sample (i.e. single cell tissue) from patient. Coat the microfluidic chip, and assemble the microfluidic chip and holder. Connect microfluidic tubing and fitting to an output (e.g., multiwall plate, 15 mL Eppendorf, etc.) for the MOSs and the waste oil.

Run the device to form the MOSs. Remove the output (e.g., plate, Eppendorf tube, etc.) containing the droplets from the incubator (after at least 15 minutes). Remove any excess oil from the output. The droplets should be buoyant, so the oil should be at the bottom of the vial. Be careful not to remove the droplets from the tube. Add 100 uL of 10% (v/v) PFO to the output. Carefully swirl and wait ˜1 min. Do not pipette or disturb the sample. Centrifuge at 300 g for 60 sec. Remove the supernatant (excess oil/PFO). Do not pipette or disturb the sample. Remove as much of the PFO as possible, as this chemical can reduce cell viability during culture. Add 1 mL of cell culture media. Do not pipette or disturb the sample. Centrifuge at 300 g for 60 sec. Remove supernatant and any excess oil/PFO. Add 1 mL of cell culture media. Carefully pipette the sample up and down (˜30 times) with a 1 mL pipette tip. Be careful not to over pipette or disturb the droplet sample. Using a 1 mL pipette tip, place the droplet-media solution through the 70 um or 100 um filter (connected to a 50 ml conical). Some droplets will stick to the inside of the output (e.g., a 15 mL Eppendorf). Rinse each tube with 2-3 mL of PBS and pipette up and down. Place rinsed PBS and droplets through the filter. Repeat this step twice, or until the tube looks clear, and the droplets have been transferred to the filter. Using a 1 mL pipette tip, carefully wash the filter containing the droplets with ˜5 mL of PBS. Try and cover the entire surface area of the filter. This washing step removes any excess oil and PFO from the sample, and allows the final recovery of the gel droplets into cell culture media.

Once drained correctly (˜1-2 minutes), carefully remove the filter from the 50 mL conical. Flip the filter upside-down and wash the back side with fresh cell culture media, and catch the solution in a fresh petri dish. This detaches the droplets from the filter, and places them in the cell culture media. It is recommended to use a 1 mL pipette tip, and wash with ˜5 mL of media

Check the quality of the droplets under the microscope. Most/all of the oil should be removed. If poor recovery, the sample can be re-filtered. Density of MOSs recovered may be checked by hemocytometer

### Example 6: Renal Tissue MOSs

In another example, MOSs may be formed from biopsied renal tissue. For example, instruments used may include: a tube rotator or 100 μm and 70 μm cell strainer, 15 ml conical tubes, 50 ml conical tubes, Razor blades, Tweezers and surgical scissors, Petri dish (100×15 mm) or tissue culture dish. The reagents may include: EBM-2 media, Collagenase (5 mg/mL stock), Hank's Balanced Salt Solution (HBSS), Calcium Chloride (10 mM stock solution), Phosphate Buffer Solution (1×PBS), MATRIGEL, 0.4% Trypan Blue solution and Trypsin.

Rental tissue to be stored in a cold transport media and on ice at all times. 2 mL of enzymatic digestion solution may be placed in a 15 mL conical tube. Add 600 uL of calcium chloride (final concentration: 3 mM) and add 200 uL of collagenase (final: 0.5 mg/mL). Transfer the renal sample into a petri/culture dish. Remove all excess or non-tumor tissue with sterile tissue or razor blade. Add 1 mL of the enzymatic solution to the tissue. Mince the sample into small pieces with the sterile razor blade (<2 mm2). Hold down the plate with tweezers or by hand. Transfer minced tissue and enzymatic solution back into the 15 mL tube with the enzymatic solution. Place the tube in the tube rotator or a 15 mL tube rotator between 30-60 minutes in 37° C. incubator. Remove the tube from the incubator. Quench the enzymatic digestion with at least 6 mL EBM-2 (at least 3 times the amount of enzymatic digestion solution). Pipette to mix. Place a 100 μm or 70 μm cell strainer onto a 50 ml conical tube. Transfer sample through the strainer. Transfer solution to a new 15 ml conical tube. Centrifuge the sample at 1500 rpm for 5 minutes. Discard the supernatant, leaving the cell pellet. Resuspend the pellet in 1 mL EBM-2 media. Add 10 μL cell mixture to 10 μL of Trypan Blue on a piece of parafilm and transfer to a cell counting plate or hematocytometer. Calculate cell concentration (#/mL). Centrifuge at 1500 RPM for 5 minutes and discard the supernatant, leaving the pellet. Resuspend cell pellet in 50 uL of MATRIGEL per 1.25×105 cells. Perform on ice. Plate 50 uL domes of MATRIGEL-cell suspension in the center of wells in a pre-warmed 24-well flat bottom plate. Transfer the plate to a 37° C. cell incubator and incubate for at least 20 minutes. Confirm that domes are polymerized. Gently add 500 μL of prewarmed EBM-2 media down the wall of the well. Incubate in 37° C. incubator. Perform a full media change every 2 days to expand MOSs.

### Example 7: Liver Micro-Organosphere

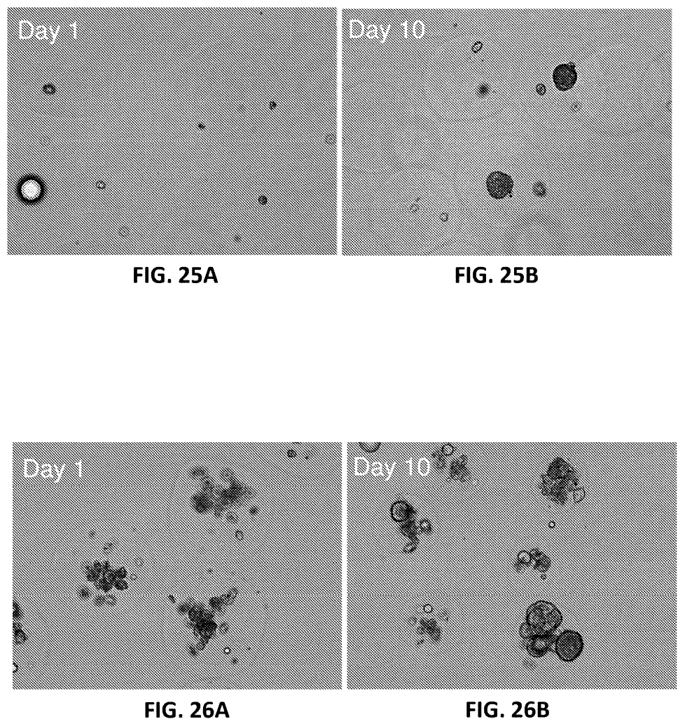

As mentioned, MOSs may be formed from normal (e.g., non-cancerous) and/or abnormal tissue. For example, FIGS. 25A-25 and 26A-26B illustrate one example of MOSs formed from a mouse liver tissue that has been dislocated, combined with a fluid matrix material to form an unpolymerized mixture, then a droplet of the unpolymerized mixture was polymerized to form the MOSs. In this example, the MOSs have a diameter of about 300 μm. In FIGS. 25A-25B the MOSs were formed with a single cell per droplet. In FIGS. 26A-26B the MOSs were formed with 25 cells per droplet. In FIG. 25A the MOSs are shown one day after forming; FIG. 25B shows the MOSs after ten days in culture. Cells in some of the MOSs have divided, forming clusters exhibiting structure; other MOSs included cells that were slower to divide or that did not divide. Similarly, in FIGS. 26A-26B the MOSs initially including about 25 cells in each MOSs. After ten days in culture, some of the MOSs showed a great deal of cell growth, forming structures, while other MOSs showed only modest growth. In both cases, the cells within the MOSs have been found to exhibit properties characteristic of the original tissue (e.g., liver cells) from which they originated.

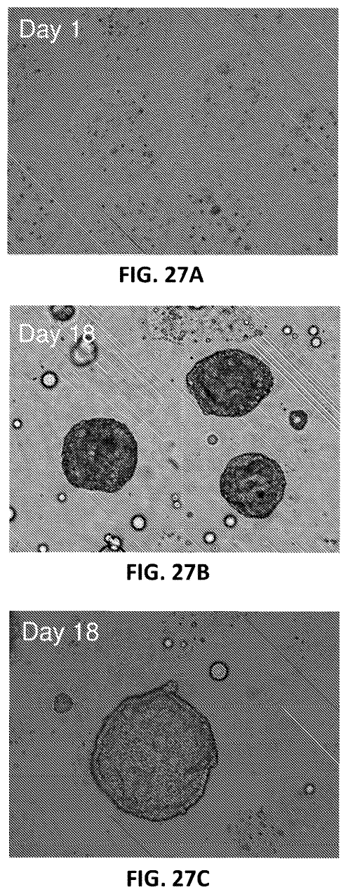

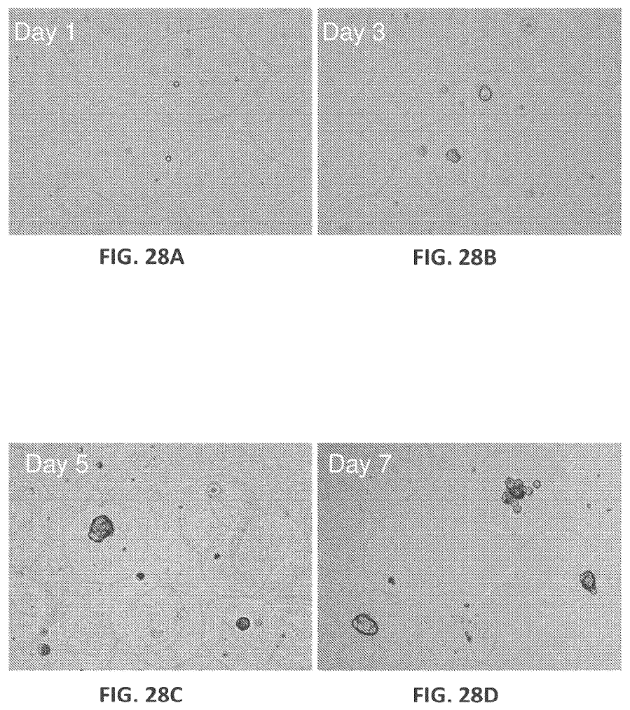

The same procedure was successfully performed on human liver tissue, as shown in FIGS. 27A-27C. In this example the MOSs were initially formed with about fifty cells, as shown in FIG. 27A. By day 18 in culture, some of the MOSs showed cells having clusters and forming structures, while others had smaller structures or the cells did not divide.

### Example 8: Cultured Cell Micro-Organospheres

In addition to primary tissues, e.g., removed from a patient immediately or shortly before forming MOSs, MOSs may be formed from cultured cells or cells, including either 2D cultured cells or 3D cultured cells.

In some embodiments, the MOSs may be formed from cell lines grown as part of a Patient Derived Xenograft (PDX). For example, FIGS. 28A-28D illustrate MOSs formed from cultured PDX240 cells. PDX240 cells are a Patient Derived Xenograft (PDX) tumor cell line (numbered 240 based on patient source) that were human tumors grown in immunodeficient mice (PDX) to form in vivo tumors. The xenograft tissue was extracted dissociated, and used to form MOSs as described above. In this example a single cell was included in each MOS as it was formed. FIG. 28A shows the MOSs after one day in culture, while FIG. 28B shows the MOSs after three days in culture and FIGS. 28C and 28D show the MOSs after five and seven days in culture, respectively. With progressive time in culture, at least some of the MOSs show the cells dividing and forming structures.

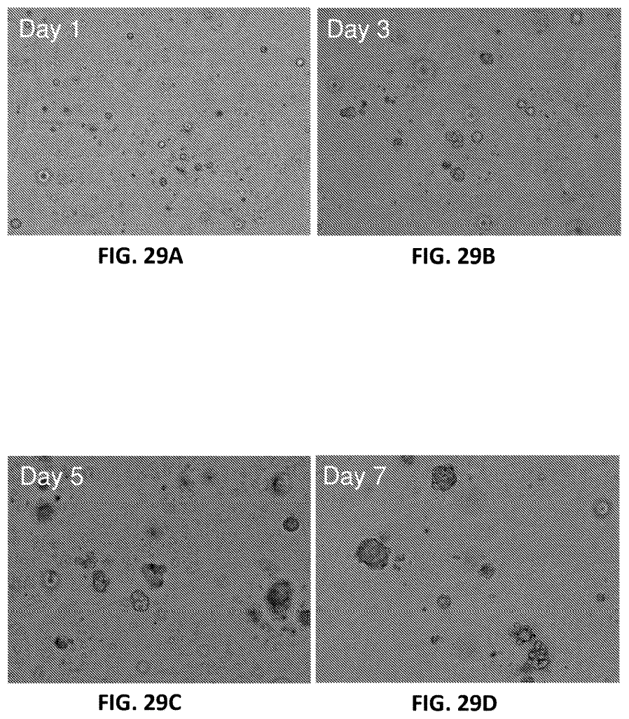

FIGS. 29A-29D show a similar experiment in which five PDX240 cells were initially included in each droplet forming each of the MOSs. With time in culture (e.g., from day 1, day 3, day 5 and day 7, as shown in FIGS. 29A-29, respectively) the cells may divide and form structures.

### Example 9: Comparison of MOSs With Traditional Organoids

Organoids were formed from Patient Derived Xenograft cells (including the PDX240 cells described above and a second PDX cell line, PDX19187) and were compared with MOSs formed using the same cells. The organoids were formed using conventional techniques in which a large mass of MATRIGEL in a well or dish was seeded with cells and cultured until growth was confirmed. MOSs were generated from the traditional organoids.

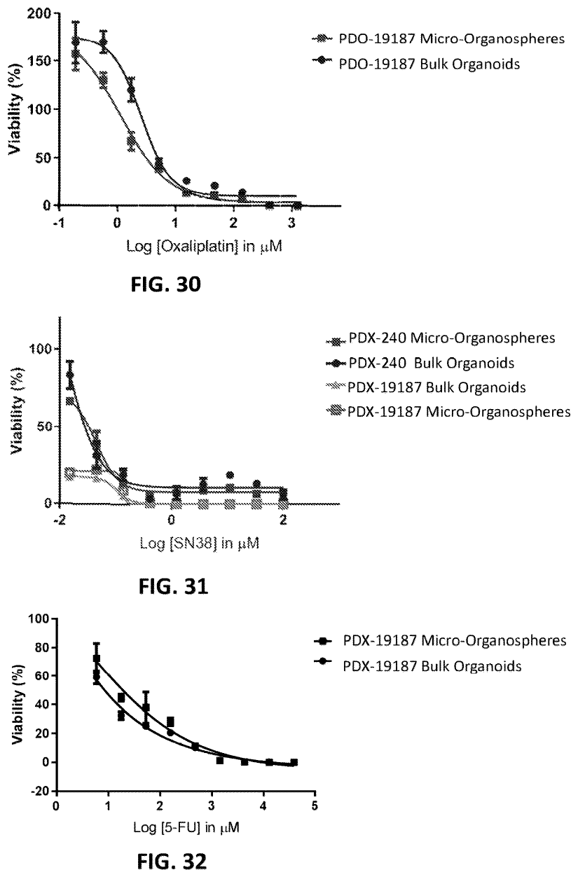

Both traditional (“bulk”) organoids and the MOSs were then treated with the same drugs (e.g., Oxaliplatin or SN38) and cell viabilities were measured after 3 days of treatment. The drug response curves shown in FIGS. 30 and 31 were generated, and show similar response curves. For example, in FIG. 30, the drug response curves of PDO19187 bulk organoids and MOSs showed similar response curves to Oxaliplatin concentration, as did PDX240 bulk organoids and MOSs. In FIG. 31, the drug response curves for both PDX19187 and PDX240 also showed similar results for both bulk organoids and MOSs for SN38. FIG. 32 shows response curves for another anti-cancer drug, 5-FU (Fluorouracil), again showing similar drug response curves for both PDZ-19187 and PDX-240 traditional organoids and MOSs.

Thus, the MOSs described herein, which may be formed more quickly and reliably, and which may have a higher overall survival rate as compared to traditional organoids, may provide drug responses that are comparable to those of bulk organoids formed using the same cells. However, as described herein, the MOSs may be used more quickly and may be formed in much larger numbers.

### Example 10: Drug Effects on MOSs

In general, the MOSs described herein may be used to perform one or more assays, including toxicity assays. Any appropriate assay may be performed, as the results determined by analysis of the tissue (e.g., cells, tissue structures) suspended within the MOSs. The MOSs described herein may be assayed or analyzed optically, chemically, electrically, genetically, or in any other manner known in the art.

Optical (either manual or automatic) detection may be particularly

useful and may include optically analyzing the effects of one or more drug formulations on the tissue (including cells, clusters of cells, structures of cells, etc.) within the MOSs. In some embodiments, as mentioned above, the drug formulation may be assayed for cell death (e.g., number and/or size of tissues) within the MOSs tested. In other embodiments, the MOSs may be assayed for cell growth, including reduction in the size, type and/or rate of growth. In some embodiments, the MOSs may be assayed for changes in the tissue structures formed.

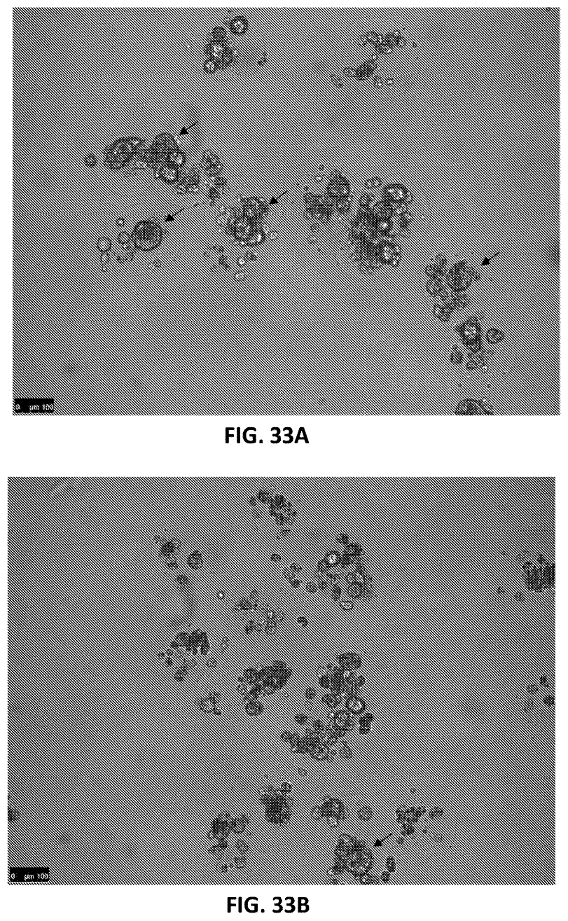

For example, FIG. 33A-33B illustrate the effect of one drug formulation, in this example, acetaminophen (10 mM) on mouse liver MOSs. FIG. 33A is a control group, in which the MOSs were not treated, showing tissue within the MOSs (arrows) grown when cultured. FIG. 33B shows a similar set of MOSs formed from mouse liver that were instead treated with 10 mM acetaminophen. In the control group the tissue structures within the MOSs are relatively large as compared with the treatment group. The tissue in most of the MOSs of the acetaminophen group is smaller and contains many dead cells.

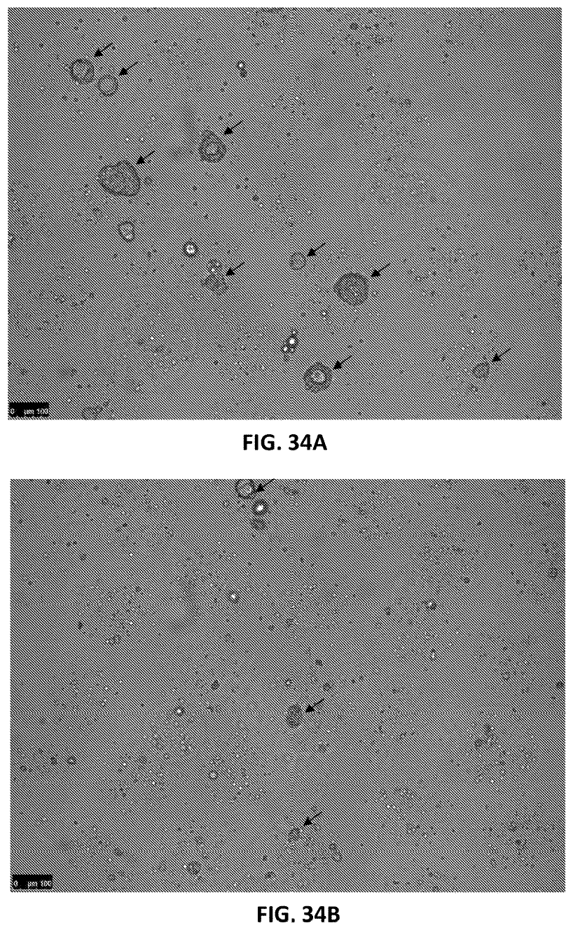

Similarly, FIGS. 34A-34B also show toxicity assays using human liver MOSs. FIG. 34A shows typical human liver MOSs observed in the control group including tissue structures (indicated by the arrows) formed therein. FIG. 34B shows the treatment group, in which the human liver MOSs are treated with acetaminophen (10 mM). The tissue in the treated MOSs showed a significant increase atypical tissue structures (arrows) and debris, as compared to the control group.

### Example 11: Rapid Microorganosphere Production (RMOS) Preserves Local Microenvironment and Spatial Heterogeneity

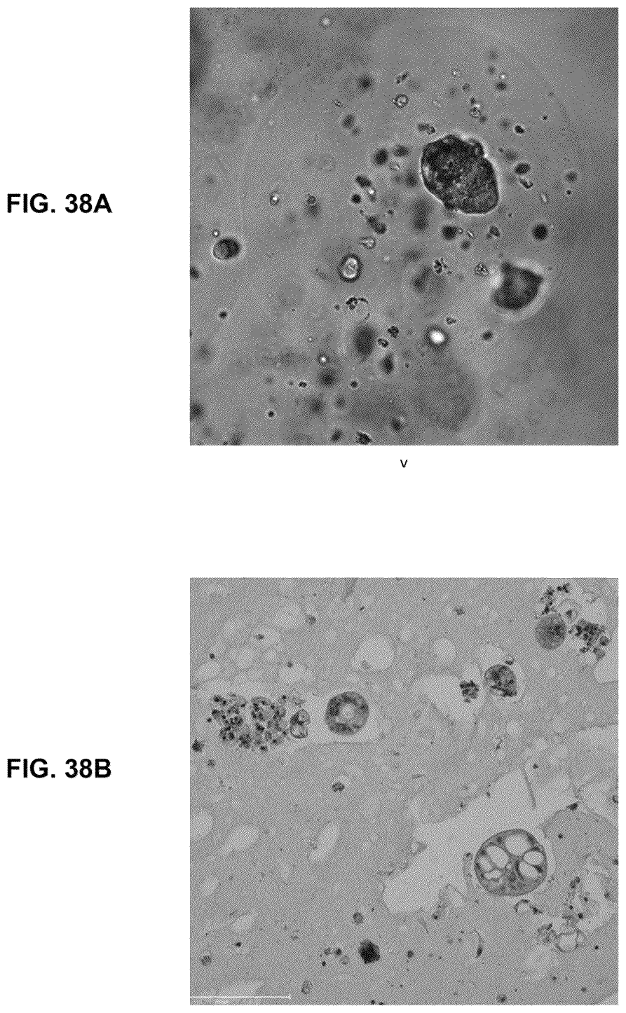

The MOS organoids (as seen in FIGS. 38A and 38B) were generated from chunk preparation (described earlier). The depiction of FIG. 38A was taken only one day after MOS generation at 20× magnification. An organoid was formed with clear boundary and structure. As seen in FIG. 38A, all the other stromal components are preserved in the MOS. The depiction of FIG. 38B was taken on day 2. The organoids in FIG. 38B were fixed in 2% PFA+1% glutaraldehyde for 30 mins at room temperature, then washed twice with 1×PBS before keeping in 70% ETOH. The organoids were embedded in paraffin and sections were stained with B cell marker CD19 on IHC. Staining results indicated that the MOS preserved immune cells (especially B cells) around the organoids.

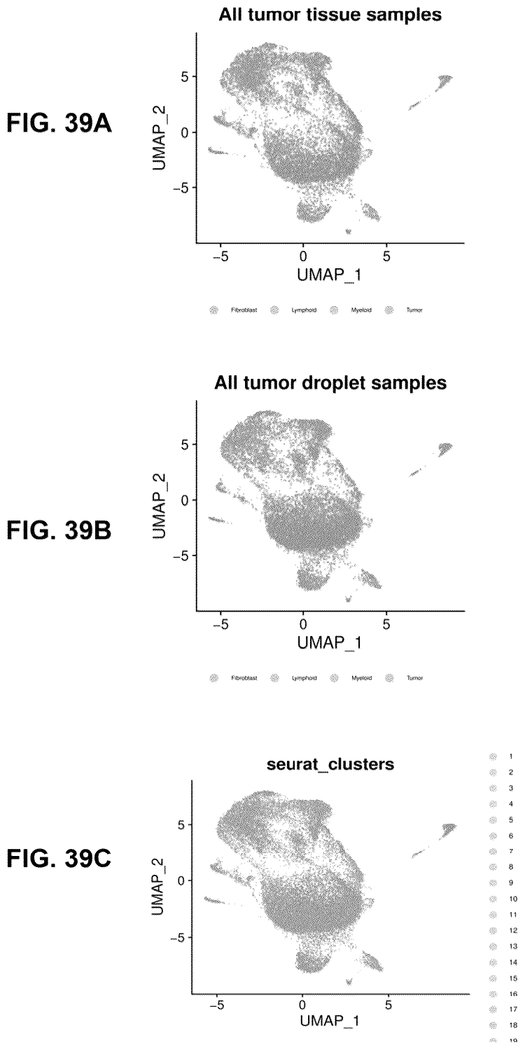

As seen in the single cell transcriptomics analysis of FIGS. 39A-39C, MOS preserves tumor stromal cells. Single cell RNA seq data was generated from 5 pairs of parent tumor tissue samples and MOS (3 lung, 1 kidney, 1 ovarian cancer) using drop-seq RNA seq technology. Single cell analysis on Uniform Manifold Approximation and Projection (UMAP) was colored by cell type and split into all tissue samples (see FIG. 39A) or droplet samples (see FIG. 39B). Cell types were summarized into one of four groups: fibroblasts; lymphoid cells (T cells, B cells, NK cells); myeloid cells (monocytes, neutrophils, DCs); tumor cells (epithelium). Comparing with UMAPs from all tissue samples, the MOS UMAPs suggested that four major types of cells were preserved in MOS. As seen in the graph-based cluster of FIG. 39C, (computed by Seurat) a total of 19 sub cell populations were identified in both tissue and MOS. This data provides evidence that MOS preserves both major and sub cell populations in tumor epithelium and stromal environment.

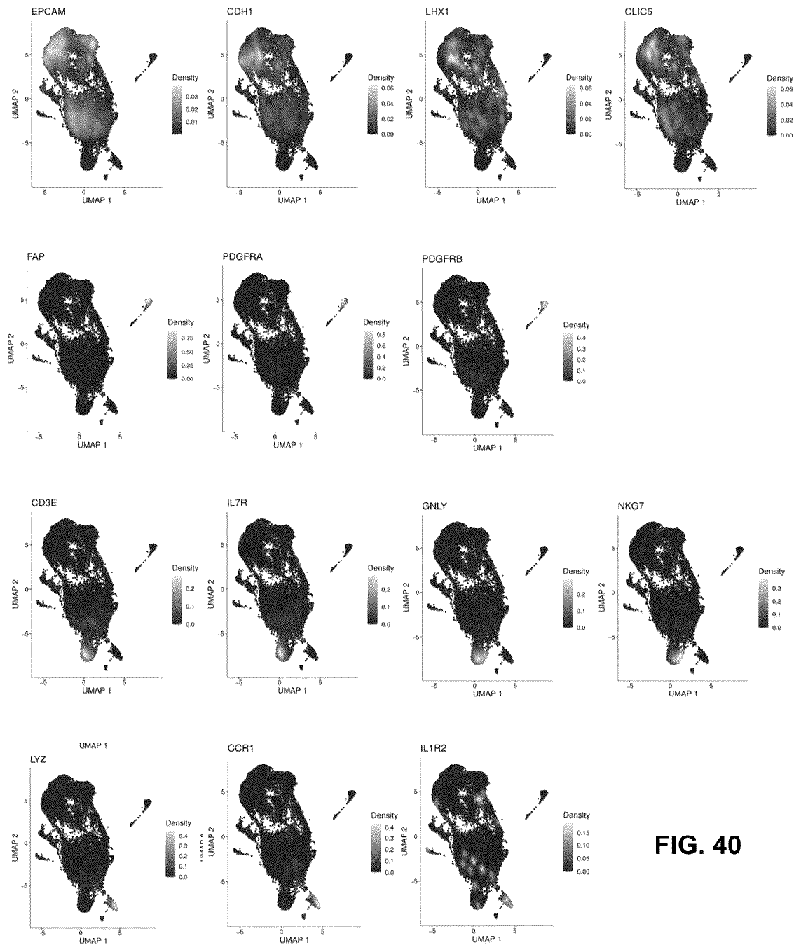

As seen in the UMAPs of FIG. 40, various markers show the viable presence of the four major types of cells were preserved in the MOSs. Tumor markers included EPCAM, CDH1, LHX1, CLIC5; Fibroblast markers included PDGFRA/B, FAP; Lymphoid markers included CD3E, IL7R, GNLY, NKG7; and Myeloid markers included LYZ, CCR1, IL1R2.

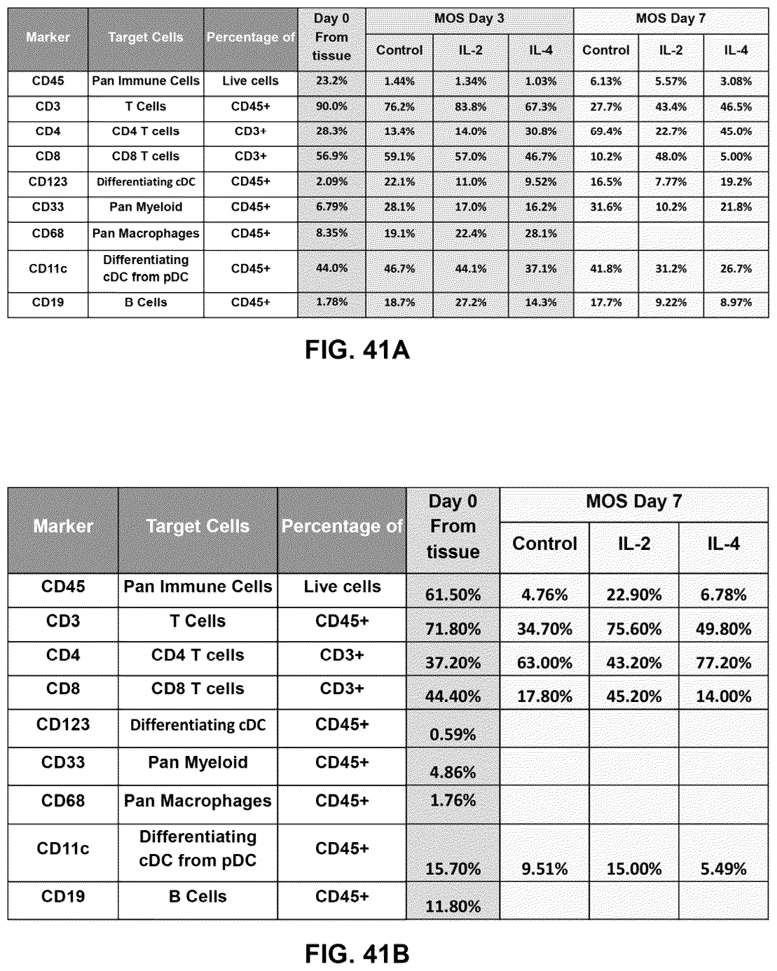

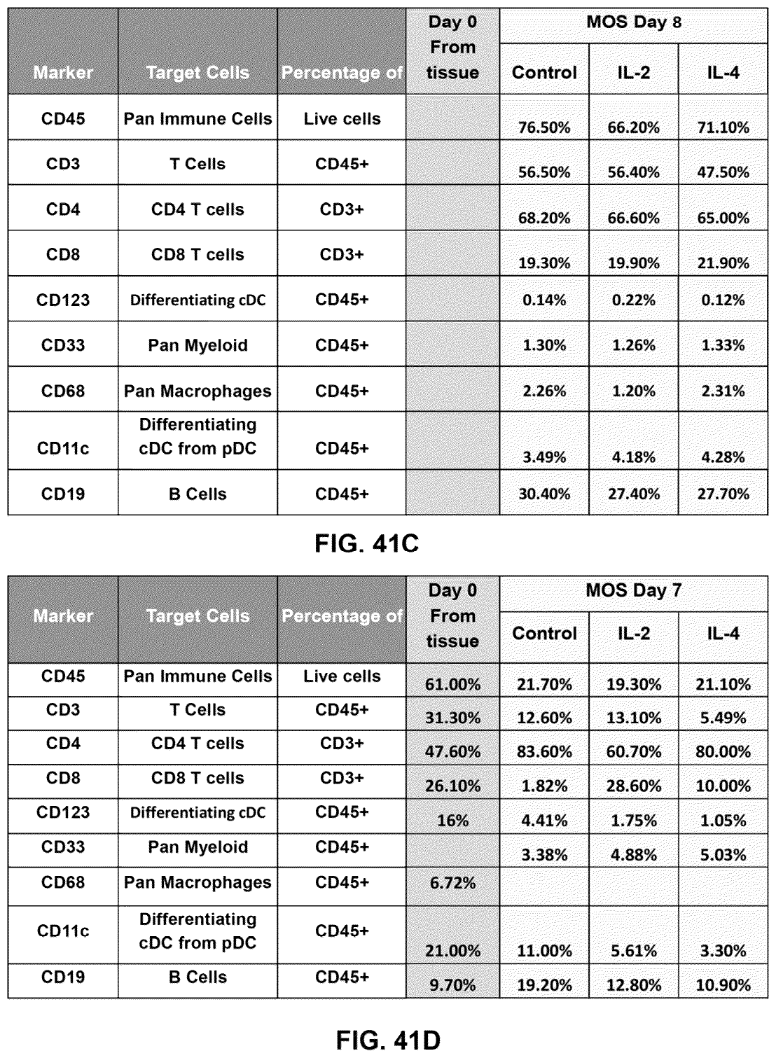

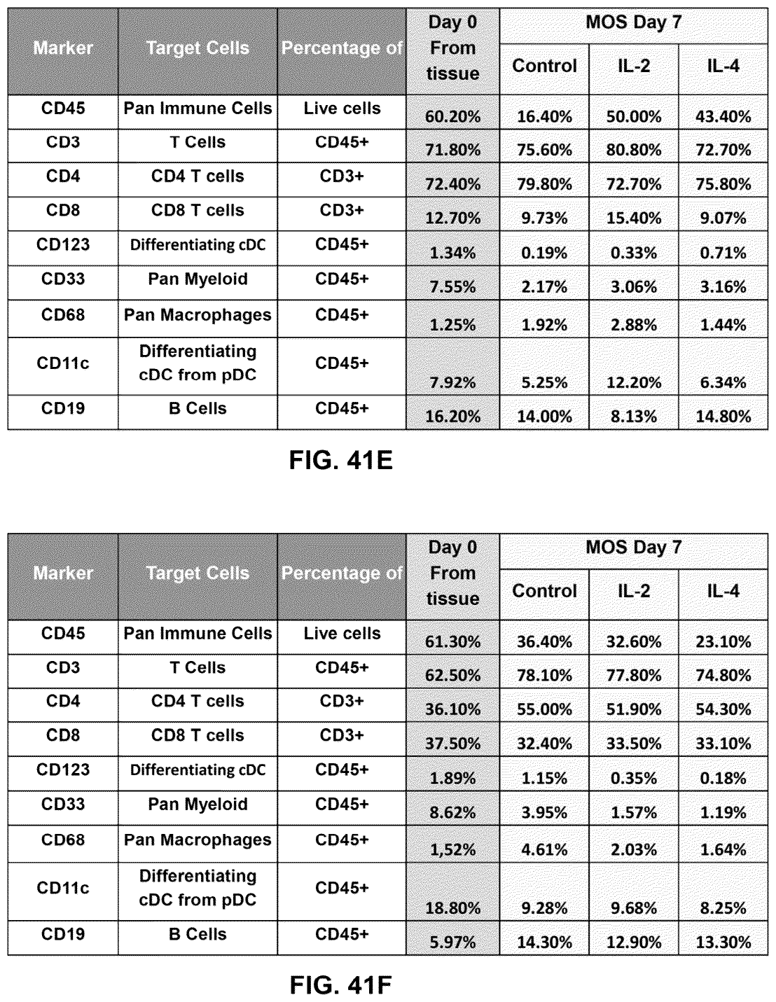

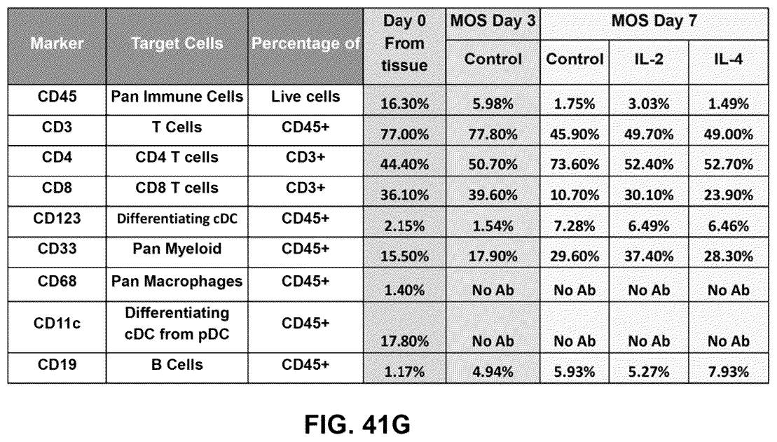

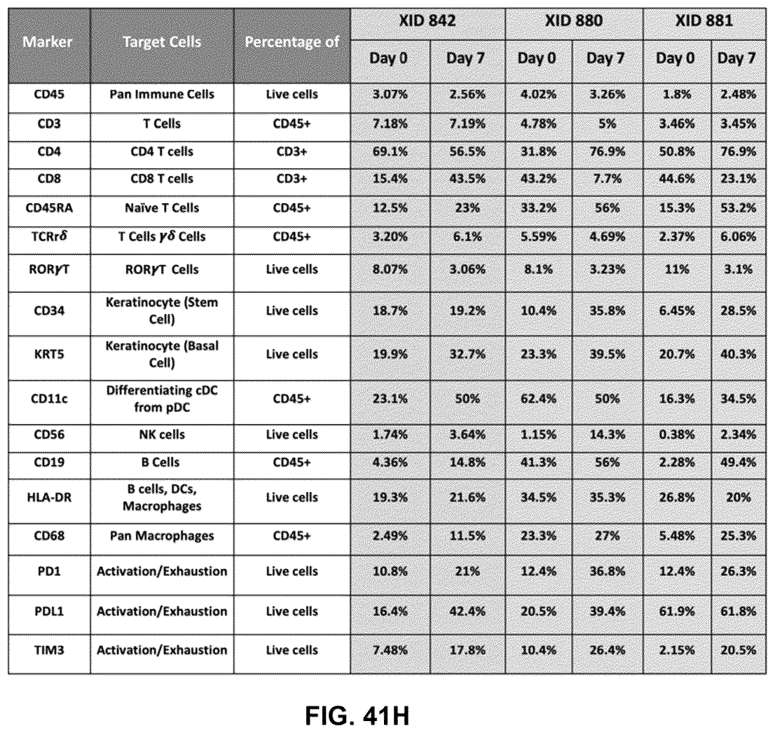

FIGS. 41A-41H show percentage viability of various immune components in MOSs (at different time periods) and biopsy tissue. FIG. 41A shows immune components preserved based on a first lung tumor sample. FIG. 41B shows immune components preserved based on a second lung tumor sample. FIG. 41C shows immune components preserved based on a first normal lung tissue sample. FIG. 41D shows immune components preserved based on a second normal lung tissue sample. FIG. 41E shows immune components preserved based on a colorectal tumor sample. FIG. 41F shows immune components preserved based on a first renal tumor sample. FIG. 41G shows immune components preserved based on a second renal tumor sample. FIG. 41H shows immune components preserved based on an autopsy skin sample. As seen in FIGS. 401-41H, embodiments described herein allow immune cells to have prolonged persistence in MOS.

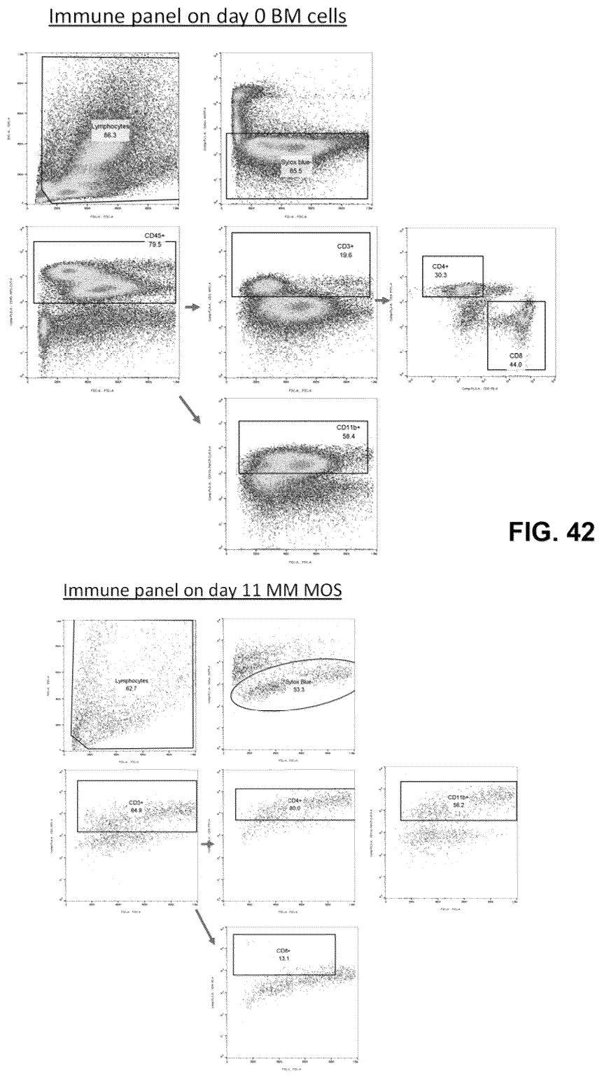

FIG. 42 illustrates the preservation of immune cells in an established multiple myeloma biopsy. As seen in FIG. 42, immune cells persist in the MOSs even into day 11.

Any of these reviews, including optical reviews, may be scored, graded, ranked, or otherwise quantified. For example, in FIGS. 33A-33B and 34A-34B, the results of these two assays may be quantified to indicate the size difference, number of live/dead cells/tissue, and the like. In some embodiments, scoring may be automated.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control/perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

Throughout this specification, unless the context requires otherwise, the word “comprise”, and embodiments such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any embodiment calculated to achieve the same purpose may be substituted for the specific embodiments shown. This disclosure is intended to cover any and all adaptations or embodiments of various embodiments. Combinations of the above embodiments, and other embodiments not specifically described herein, will be apparent to those of skill in the art upon reviewing the above description.

1. A method for testing immuno-oncology drugs and biologics using microorganospheres (MOSs), the method comprising:
a. forming a plurality of MOSs from cancerous tumor biopsy tissue, such forming comprising:
i. dissociating cells from the cancerous tumor biopsy tissue;
ii. combining the dissociated cells with a fluid matrix material;
wherein the dissociating comprises: an enzyme digestion protocol, mincing the cancerous tumor biopsy tissue, and incubating the cancerous tumor biopsy tissue in a digestion solution with agitation;
b. testing at least one immuno-oncology drug or biologic using the plurality of MOSs within 10 days after forming the plurality of MOSs.

2. The method according to claim 1 wherein the enzyme digestion protocol is collagenase based.

3. The method according to any preceding claim, wherein the plurality of MOSs comprises chunks comprising clumps of cells.

4. The method according to any preceding claim, wherein the plurality of MOSs comprises MOSs with diameters of less than 1000 μm.

5. The method according to any preceding claim, wherein the initial number of cells from the cancerous tumor biopsy tissue is between 50,000 and 500,000.

6. The method according to any preceding claim, wherein the plurality of MOSs comprises at least 500 MOSs and the cancerous tumor biopsy tissue is a single tissue biopsy.

7. The method according to any preceding claim, wherein the plurality of MOSs comprises MOSs with a range of diameters from 10 μm to 700 μm.

8. The method according to any preceding claim, wherein each MOS comprises 80 to 100 cells.

9. The method according to any preceding claim, wherein the fluid matrix material comprises a substrate basement membrane matrix.

10. The method according to any preceding claim, wherein the plurality of MOSs comprises at least 750, 1000, 2000, 5000, 10,000 or more MOSs.

11. The method according to any preceding claim, wherein the testing is carried out on day 1, day 2, day 3, day 4, day 5, day 6, day 7, day 8, day 9, or day 10 after forming the plurality of MOSs.

12. The method according to any preceding claim, wherein the fluid matrix material comprises at least one of: growth factors and structural proteins.

13. The method according to claim 12, wherein the structural proteins comprise at least one of: collagen, laminin, and nidogen.

14. The method according to any preceding claim, wherein the cancerous tumor biopsy tissue comprises at least one of: fine needle aspirate, circulating tumor cells, a liquid biopsy, and tissue from a cancer of at least one of: colon, esophagus, skin (melanoma), uterus, bone (sarcoma), kidney, ovary, lung, and breast from the primary site or metastatic sites including liver, omentum, and diaphragm.

15. The method according to any preceding claim, wherein the forming comprises maintaining and/or cryopreserving the plurality of MOSs.

16. The method according to claim 15, wherein maintaining comprises maintaining for at least one month.

17. The method according to any preceding claim, wherein the testing comprises at least one of: efficacy testing, screening, drug sensitivity tests, testing the efficacy of enhanced immune cells in vitro, rapid testing, toxicity testing and performing ex-vivo testing of drug response.

18. The method according to any preceding claim, wherein the combining of the dissociated cells with a fluid matrix material comprises forming an unpolymerized material.

19. The method according to claim 18, wherein the forming further comprises polymerizing the unpolymerized material to form a plurality of MOSs.

20. The method according to claim 19, further comprising allowing the cells to grow in the polymerized droplet.

21. An apparatus suitable for carrying out the method of any preceding claim.
